(19) United States
(12) Patent Application Publication
(10) Pub. No.: US 2024/0051950 A1
(43) Pub. Date: Feb. 15, 2024

(54) Novel CYP3A4-Specific Inhibitors and Methods of Using Same

(71) Applicant: THE REGENTS OF THE UNIVERSITY OF CALIFORNIA, oakand, CA (US)

(72) Inventors: Irina F. Sevrioukova, Irvine, CA (US); Eric R. Samuels, Long Beach, CA (US)

(21) Appl. No.: 18/258,610

(22) PCT Filed: Dec. 22, 2021
(86) PCT No.: PCT/US21/64809

§ 371 Date: Jun. 21, 2023

Related U.S. Application Data
(60) Provisional application No. 63/128,916, filed on Dec. 22, 2020.

(51) Int. Cl. C07D 405/12; A61K 45/06; C07D 401/14; C07D 213/82

(52) U.S. Cl. CPC C07D 405/12; A61K 45/06; C07D 401/14; C07D 213/82

(57) ABSTRACT

The present invention provides novel compounds and compositions thereof inhibiting cytochrome P450 3A4 (CYP3A4). The present invention further provides a novel method of inhibiting CYP3A4 in a subject in need thereof, the method comprising administering to the subject an effective amount of a compound of the invention. In one embodiment, the subject is further administered at least one additional therapeutic agent.

### CROSS-REFERENCE TO RELATED APPLICATIONS

This application claims priority under 35 U.S.C. § 119(e) to U.S. Provisional Patent Application Ser. No. 63/128,916, filed Dec. 22, 2020, the disclosure of which is incorporated herein by reference in its entirety.

### STATEMENT REGARDING FEDERALLY SPONSORED RESEARCH OR DEVELOPMENT

This invention was made with Government support under Grand No. ES025767, awarded by the National Institutes of Health (NIH). The Government has certain rights in the invention.

### BACKGROUND OF THE INVENTION

Human cytochrome P450 3A4 (CYP3A4) is the major and most clinically relevant drug-metabolizing enzyme and clears ˜60% of prescribed pharmaceuticals via oxidation. One way to increase plasma levels and improve therapeutic efficiency of the quickly metabolized drugs is through controlled CYP3A4 inhibition. Currently, two potent CYP3A4 inhibitors, ritonavir and cobicistat, are marketed as boosters for anti-HIV and anti-HCV drugs whereas ketoconazole is widely used to slow down the CYP3A4-mediated cyclosporine metabolism and reduce the cost of immunosuppressive therapy in organ transplant recipients.

Ritonavir was originally developed as an HIV protease inhibitor and its CYP3A4 inhibitory activity was completely accidental. Cobicistat, a derivative of ritonavir lacking the anti-HIV activity, was also developed based on the chemical structure/activity relationship (SAR) studies rather than the crystal structure of CYP3A4. Cobicistat has better physico-chemical properties and causes fewer side effects but inhibits CYP3A4 less potently than ritonavir and has undesired off target activities. Ketoconazole, on the other hand, is an anti-fungal drug that causes severe liver injuries and adrenal gland problems and, as a consequence, the FDA now limits its usage.

Thus, there is a need in the art to identify novel compounds which inactivate CYP3A4 potently and specifically. The present invention fulfills this need.

### SUMMARY OF THE INVENTION

In one aspect, the present invention provides a compound having the structure

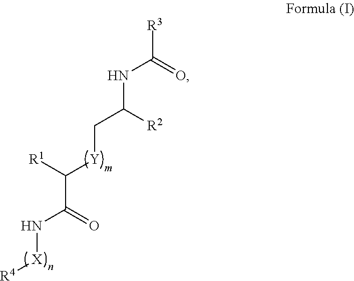

or a salt or solvate thereof, and any combinations thereof.

In some embodiments, R₁ and R₂ are each independently selected from H, C₁-C₆ alkyl, substituted C₁-C₆ alkyl, aryl, phenyl, substituted phenyl, heteroaryl, substituted heteroaryl, heterocyclyl, substituted heterocyclyl, —(C₁-C₆)alkyl-aryl, substituted —(C₁-C₆)alkyl-aryl, —(C₁-C₆)alkyl-phenyl, substituted —(C₁-C₆)alkyl-phenyl, —(C₁-C₆)alkyl-carbocyclic, —(C₁-C₆)alkyl-heteroaryl, substituted —(C₁-C₆)alkyl-heteroaryl, N(R₅)(R₆), or any combination thereof. In one embodiment, R₁ is benzyl. In some embodiments, R₂ is —(C₁-C₆)alkyl-aryl or —(C₁-C₆)alkyl-heteroaryl.

In some embodiments, R₃ is heteroaryl, substituted heteroaryl, heterocyclyl, substituted heterocyclyl, —(C₁-C₆)alkyl-NHC(═O)R₇, or any combination thereof. In some embodiments, R₃ is furan, thiophene, benzofuran, benzothiophene, or pyridine.

In some embodiments, R₄ is heteroaryl, substituted heteroaryl, heterocyclyl, substituted heterocyclyl, or any combination thereof. In one embodiment, R₄ is pyridine.

In some embodiments, R₅ and R₆ are each independently selected from H, C₁-C₆ alkyl, substituted C₁-C₆ alkyl, aryl, phenyl, substituted phenyl, heteroaryl, substituted heteroaryl, heterocyclyl, substituted heterocyclyl, —(C₁-C₆)alkyl-aryl, substituted —(C₁-C₆)alkyl-aryl, —(C₁-C₆)alkyl-phenyl, substituted —(C₁-C₆)alkyl-phenyl, —(C₁-C₆)alkyl-carbocyclic, —(C₁-C₆)alkyl-heteroaryl, substituted-(C₁-C₆)alkyl-heteroaryl, or any combination thereof.

In some embodiments, R₇ is phenyl, substituted phenyl, heteroaryl, substituted heteroaryl, heterocyclyl, substituted heterocyclyl, or any combination thereof. In one embodiment, R₇ is pyridine.

In some embodiments, each occurrence of Y is independently selected from CH₂, NH, S, or O. In one embodiment, Y is S.

In some embodiments, each occurrence of X is independently selected from —(C₁-C₆) alkyl or —(C₁-C₆) alkyl-O—(C₁-C₆) alkyl-.

In some embodiments, m is an integer 0 or 1. In one embodiment, m is 1.

In some embodiments, n is an integer from 0 to 3. In one embodiment, n is 1.

In some embodiments, the compound of the present invention is

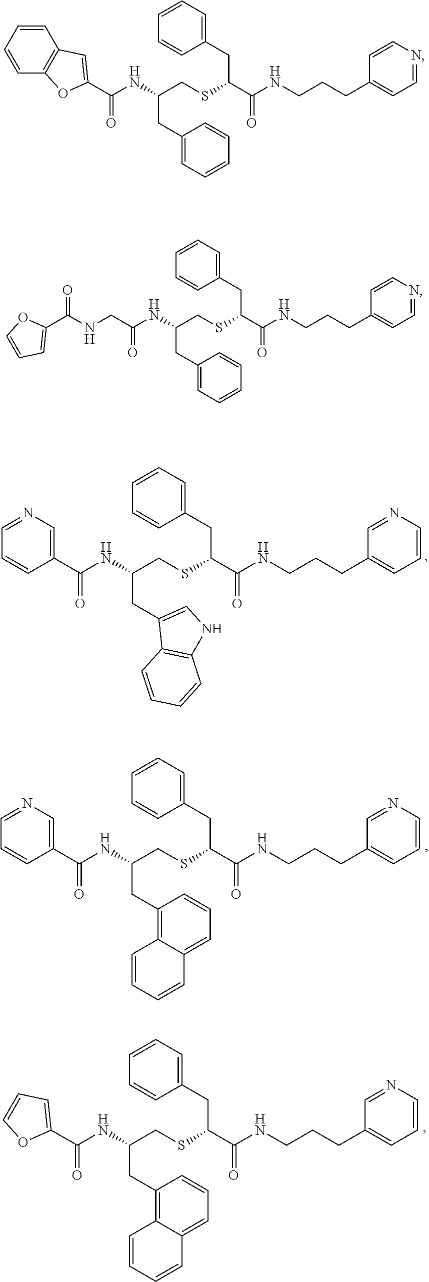

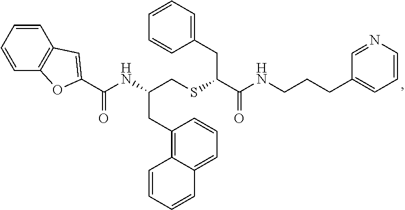 [SMILES: O=C(N[C@H](CS[C@H](Cc1ccccc1)C(=O)NCCCc1cccnc1)Cc1cccc2ccccc12)c1cc2ccccc2o1]

or a salt or solvate thereof, and any combinations thereof.

In one aspect, the present invention provides a composition comprising at least one compound described herein.

In some embodiments, the composition further comprises a pharmaceutically acceptable carrier.

In one aspect, the present invention provides a method of inhibiting at least one cytochrome P450 3A4 (CYP3A4) in a subject in need thereof.

In one aspect, the present invention provides a method of treating or preventing at least one disease or disorder associated with CYP3A4 in a subject in need thereof.

In some embodiments, the method comprises administering to the subject a therapeutically effective amount of at least one compound described herein or a composition thereof.

In some embodiments, the method further comprises administering to the subject at least one additional therapeutic agent. In some embodiments, the therapeutic agent is an antiviral agent, anti-cancer agent, immunosuppressant agent, or any combination thereof. In one embodiment, the therapeutic agent is a protease inhibitor.

In some embodiments, the composition and the additional therapeutic agent are co-administered. In some embodiments, the composition and the additional therapeutic agent are co-formulated.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts the CYP3A4 inhibitors of the present invention.

FIG. 2 depicts the chemical structures of ritonavir, cobicistat, and ketoconazole.

FIG. 3, comprising FIG. 3A through FIG. 3C, depicts data for inventive compound 04ERS022. FIG. 3A depicts the calculation of spectral K_{d} (dissociation constant) from equilibrium titration plots. FIG. 3B depicts the calculation of IC₅₀ (half maximal inhibitory concentration) from plots of % activity versus inhibitor concentration. FIG. 3C depicts the heme-ligated 04ERS022 in the co-crystal structure with CYP3A4.

FIG. 4, comprising FIG. 4A through FIG. 4C, depicts schemicatic representations of chemical structures of ritonavir, a highly potent CYP3A4 inhibitor in clinical use, and two best rationally designed inhibitors (Samuels E R et al., 2020, Bioorg. Med. Chem., 28:115349-115360). The R₁ and R₂ side-groups are indicated. If the central hydroxyl group of ritonavir was removed, the phenyl side-groups were in R/R configuration.

FIG. 4A depicts a schematic representation of the chemical structure of ritonavir, a highly potent CYP3A4 inhibitor in clinical use. FIG. 4B depicts a schematic representation of a chemical structure of rationally designed CYP3A4 inhibitor (Samuels E R et al., 2020, Bioorg. Med. Chem., 28:115349-115360). FIG. 4C depicts a schematic representation of a chemical structure of rationally designed CYP3A4 inhibitor (Samuels E R et al., 2020, Bioorg. Med. Chem., 28:115349-115360).

FIG. 5 depicts schematic representations of chemical structures of compounds 3a-i, 4e-h, 6, and 8 of the present invention.

FIG. 6, comprising FIG. 6A through FIG. 6J, depicts representative spectral properties and representative binding modes of compounds 3a-d and 8, shown in FIG. 5. Absorbance spectra of ferric ligand-free and inhibitor-bound CYP3A4 are in black and red, respectively. Spectra of ferrous ligand-bound CYP3A4 and its CO-adduct are in green and blue, respectively. Left insets are difference spectra recorded during equilibrium titrations; right insets are titration plots with quadratic fittings. The derived spectral dissociation constants (Kₛ) are given in FIG. 8. The adjacent I-helix and F304 in the inhibitory complexes and water-bound CYP3A4 (5VCC structure) are depicted in gray and black, respectively. Polder omit maps are contoured at 3σ level and shown as green mesh.

FIG. 6A depicts representative spectral properties and the binding modes of compounds 3a.

FIG. 6B depicts representative spectral properties and the binding modes of compounds 3b.

FIG. 6C depicts representative spectral properties and the binding modes of compounds 3c.

FIG. 6D depicts representative spectral properties and the binding modes of compounds 3d.

FIG. 6E depicts representative spectral properties and the binding modes of compounds 8.

FIG. 6F representative binding modes of 3a observed in the crystal structures. FIG. 6G representative binding modes of 3b observed in the crystal structures. FIG. 6H representative binding modes of 3c observed in the crystal structures. FIG. 6I representative binding modes of 3d observed in the crystal structures. FIG. 6J representative binding modes of 8 observed in the crystal structures. The R/S conformer of 8 (rac, S) was selectively co-crystallized with CYP3A4.

FIG. 7 depicts representative properties of series V inhibitors.

FIG. 8 depicts representative structural features of CYP3A4-inhibitor complexes.

FIG. 9, comprising FIG. 9A and FIG. 9B, depicts representative structural overlay of compounds 3a-d- and 8-bound CYP3A4 and representative comparison of the isosteric compound 4f (series IV analogue; Samuels E R et al., 2020, Bioorg. Med. Chem., 28:115349-115360) and compound 3a. FIG. 9A depicts representative structural overlay of compounds 3a-d- and 8-bound CYP3A4. Unlike other compounds, compound 3b (S, R) had a less favorable reverse side-group orientation, with R₂-phenyl above the I-helix (P1 site) and R₂-phenyl near the heme-ligating pyridine (P2 site). FIG. 9B depicts representative comparison of the isosteric compound 4f (series IV analogue; Samuels E R et al., 2020, Bioorg. Med. Chem., 28:115349-115360) and compound 3a demonstrating how one-atom linker elongation impacted the ligand binding mode.

FIG. 10 depicts representative spectral changes in CYP3A4 induced by compound 6 binding. Absorbance spectra of ferric ligand-free and inhibitor-bound CYP3A4 are in black and red, respectively. Spectra of ferrous ligand-bound CYP3A4 and its CO-adduct are in green and blue, respectively. Left inset shows difference spectra recorded during equilibrium titrations; right inset is a titration plot with quadratic fitting. The derived Kₛ value is given in FIG. 7. It was not possible to determine the inhibitory complex structure because compound 6 dissociates from CYP3A4 during crystallization.

FIG. 11, comprising FIG. 11A through FIG. 11J, depicts representative spectral properties and binding orientations of compounds 3e-I, shown in FIG. 5. Absorbance spectra of ferric ligand-free and inhibitor-bound CYP3A4 are in black and red, respectively. Spectra of ferrous ligand-bound CYP3A4 and its CO-adduct are in green and blue, respectively. Left insets are difference spectra recorded during equilibrium titrations; right insets are titration plots with quadratic fittings. The derived Kₛ values are listed in FIG. 7. The adjacent I-helix and F304 in the inhibitory complexes and water-bound CYP3A4 (5VCC structure) are depicted in gray and black, respectively. Polder omit maps are contoured at 36 level and shown as green mesh. FIG. 11A depicts representative spectral changes induced in CYP3A4 by compound 3e. FIG. 11B depicts representative spectral changes induced in CYP3A4 by compound 3f. FIG. 11C depicts representative spectral changes induced in CYP3A4 by compound 3g. FIG. 11D depicts representative spectral changes induced in CYP3A4 by compound 3h. FIG. 11E depicts representative spectral changes induced in CYP3A4 by compound 3i. FIG. 11F depicts representative binding modes of compound 3e observed in the crystal structures. FIG. 11G depicts representative binding modes of compound 3f observed in the crystal structures. FIG. 11H depicts representative binding modes of compound 3g observed in the crystal structures. FIG. 11I depicts representative binding modes of compound 3h observed in the crystal structures. FIG. 11J depicts representative binding modes of compound 3i observed in the crystal structures.

FIG. 12, comprising FIG. 12A through FIG. 12C, depicts representative structural overlay of compounds 3e-i-bound complexes, representative superposition of the isosteric compound 7d (series IV analogue; Samuels E R et al., 2020, Bioorg. Med. Chem., 28:115349-115360) and compound 3f, and representative comparison of the best series IV and V inhibitors, compound 5b (Samuels E R et al., 2020, Bioorg. Med. Chem., 28:115349-115360) and compound 3h. FIG. 12A depicts representative structural overlay of compounds 3e-i-bound complexes. Unlike compound 3b (S, R) (FIG. 9A), compound 3f (S, R) bound in a traditional orientation. Even so, compound 3f formed the longest Fe—N bond and could not establish an H-bond with the active site S119 (FIG. 8), meaning that the R/S configuration was still less favorable. FIG. 12B depicts representative superposition of the isosteric compound 7d (series IV analogue; Samuels E R et al., 2020, Bioorg. Med. Chem., 28:115349-115360) and compound 3f demonstrating a small impact of one-atom linker elongation on the ligand binding mode. FIG. 12C depicts representative comparison of the best series IV and V inhibitors, compound 5b (Samuels E R et al., 2020, Bioorg. Med. Chem., 28:115349-115360) and compound 3h. The pyridyl-ethyl containing compound 5b had a five- rather than four-atom R₁-R₂ spacer (FIG. 4B) and adopted a distinct conformation in the active site: R₁-phenyl protruded deeper into the P1 pocket, R₂-naphthalene was vertical rather than horizontal relative to the heme plane, and the terminal Boc-group pointed away rather than toward the substrate channel.

FIG. 13, comprising FIG. 13A and FIG. 13B, depicts representative H₂O₂ dependent heme depletion in ligand-free and inhibitor-bound CYP3A4. FIG. 13A depicts representative results demonstrating the effect of series V inhibitors on the heme accessibility to H₂O₂. Overall, the inhibitor binding significantly protected the cofactor, and the amount of heme destroyed correlated with the Kₛ/IC₅₀ values. FIG. 13B depicts a representative comparative assay for CYP3A4 bound to ritonavir and the best series IV inhibitors, compounds 5b and 7d. The heme accessibility was the lowest in CYP3A4 bound to the high affinity/potency inhibitors, compounds 3h and 7d.

FIG. 14 depicts a schematic representation of a general procedure for synthesis of compounds 3a-i.

FIG. 15 depicts a schematic representation of a general procedure for synthesis of compounds 4-6.

FIG. 16 depicts a schematic representation of a general procedure for synthesis of compounds 7 and 8.

FIG. 17, comprising FIG. 17A through FIG. 17D, depicts representative packing and spatial arrangements of 3a-, 3e- and 3i-bound CYP3A4. FIG. 17A depicts representative restructuring of the F-F′ helix/loop region triggered by distinct crystal packing in the C2 space group. The F-F′-G′-G fragments of ligand-free (5VCE; 1222) and 3a-, 3e- and 3i-bound CYP3A4 (C₂) are shown in black, red, beige and purple, respectively. FIG. 17B depicts representative crystallographic dimer of CYP3A4 and its spatial arrangement in C2 crystals, respectively. The 400-430 fragment (in green and magenta) is at the monomer-monomer interface, whereas the F-G fragment (in black and purple) mediates inter-dimer contacts. FIG. 17C depicts representative crystallographic dimer of CYP3A4 and its spatial arrangement in C2 crystals, respectively. The 400-430 fragment (in green and magenta) is at the monomer-monomer interface, whereas the F-G fragment (in black and purple) mediates inter-dimer contacts. FIG. 17D depicts representative view at the proximal face of CYP3A4. The water-bound protein (5VCC structure) is in gray; 3a- and 3e-bound complexes of WT CYP3A4 are in pink and light blue, respectively; and 3i-bound K421A/K424A CYP3A4 is in black. Structural superposition shows that substitution of surface K421 and K424 with alanine does not distort the proximal loop region. Non-mutated lysine side chains are displayed.

FIG. 18, comprising FIG. 18A through FIG. 18D, depicts representative binding of 3a and 3h to the active site of CYP3A4 crystallized in I222 and C2 space groups. For both 3a and 3h, electron density maps were not clearly defined in the I222 structures (green mesh in FIG. 18A and FIG. 18C, respectively). Therefore, 3a- and 3h-bound CYP3A4 was recrystallized in a more densely packed C2 space group (FIG. 18B and FIG. 18D, respectively). FIG. 18A depicts representative binding of 3a to the active site of CYP3A4 crystallized in I222 space groups. FIG. 18B depicts representative binding of 3a to the active site of CYP3A4 crystallized in C2 space groups. Comparison of the ligand binding modes observed in the I222 and C2 structures shows that there are virtually no primarily imposed by changes in the F-F′ loop (residues 210-213) for 3a. FIG. 18C depicts representative binding of 3h to the active site of CYP3A4 crystallized in I222 space groups. FIG. 18D depicts representative binding of 3h to the active site of CYP3A4 crystallized in C2 space groups. Comparison of the ligand binding modes observed in the I222 and C₂ structures shows that there are only minor distortions primarily imposed by changes in the F-F′ loop (residues 210-213) for 3h.

FIG. 19, comprising FIG. 19A through FIG. 19C, depicts representative spectral and ligand-binding properties of K421A/K424A CYP3A4. Absorbance spectra of ferric ligand-free and inhibitor-bound CYP3A4 (2 μM) were recorded in 0.1 M phosphate buffer, pH 7.4, supplemented with 20% glycerol and 1 mM dithiothreitol, and displayed in black and red, respectively. Spectra of the ferrous form and its CO-adduct are in green and blue, respectively. The inhibitor concentration was 10 μM. Equilibrium titrations were conducted as described below. Titration plots with quadratic fittings are shown in insets. FIG. 19A depicts representative spectral changes induced in the mutant by 3d. The derived dissociation constant was 0.019 μM for 3d. FIG. 19B depicts representative spectral changes induced in the mutant by 3i. The derived dissociation constant was 0.025 μM for 3i. FIG. 19C depicts representative spectral changes induced in the mutant by ritonavir. The derived dissociation constant was 0.023 μM for ritonavir.

FIG. 20 depicts representative data collection and refinement statistics.

FIG. 21 depicts representative data collection and refinement statistics.

FIG. 22, comprising FIG. 22A through FIG. 22K, depicts representative ¹H nuclear magnetic resonance (NMR) spectra of the compounds of the present invention.

FIG. 23, comprising FIG. 23A through FIG. 23K, depicts representative mass spectrometry (MS) spectra of the compounds of the present invention.

### DETAILED DESCRIPTION OF THE INVENTION

This invention includes the identification of novel compounds that are useful for inhibition of cytochrome P450 3A4 (CYP3A4). As demonstrated herein, the compounds of the present invention have been shown to be effective inhibitors of CYP3A4.

Human CYP3A4 is a key xenobiotic-metabolizing enzyme that oxidizes and clears the majority of drugs. CYP3A4 inhibition may be beneficial and enhance therapeutic efficiency of co-administered pharmaceuticals that are quickly metabolized by CYP3A4. Based on investigations of analogs of ritonavir, a potent CYP3A4 inactivator and pharmacoenhancer, a pharmacophore model has been built for a CYP3A4-specific inhibitor. A large set of rationally designed compounds was synthesized to test this model. The functional and structural data presented herein agree well with the proposed pharmacophore. In particular, the importance of a flexible backbone, the H-bond donor/acceptor moiety and aromaticity of the side groups analogous to Phe-1 and Phe-2 of ritonavir were confirmed, and the leading role of hydrophobic and aromatic interactions at the sites adjacent to the heme and phenylalanine cluster in the ligand binding process was demonstrated. The X-ray structures of CYP3A4 bound to the rationally designed inhibitors provide deeper insights into the mechanism of the CYP3A4-ligand interaction. Most importantly, compounds of the present invention are less complex than ritonavir, have higher affinity and inhibitory potency for CYP3A4 and, thus, could be used in the present form or serve as templates for synthesis of the next generation of inhibitors for further improvement of the binding and inhibitory strength.

The present invention also includes novel methods of inhibiting CYP3A4 in a patient in need thereof using the compounds of the invention.

The present invention also includes a composition comprising at least one compound of the invention, wherein the composition optionally further comprises at least one additional therapeutic agent. In one embodiment, the additional therapeutic agent is an antiviral agent.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal,” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a sign or symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the terms “effective amount,” “pharmaceutically effective amount” and “therapeutically effective amount” refer to a nontoxic but sufficient amount of an agent to provide the desired biological result. That result may be reduction and/or alleviation of a sign, a symptom, or a cause of a disease or disorder, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

An “effective amount” of a delivery vehicle is that amount sufficient to effectively bind or deliver a compound.

As used herein, the term “potency” refers to the dose needed to produce half the maximal response (ED₅₀).

As used herein, the term “efficacy” refers to the maximal effect (Eₘₐₓ) achieved within an assay.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

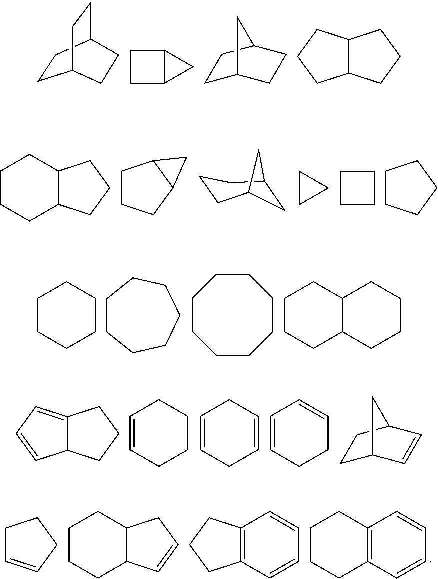

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

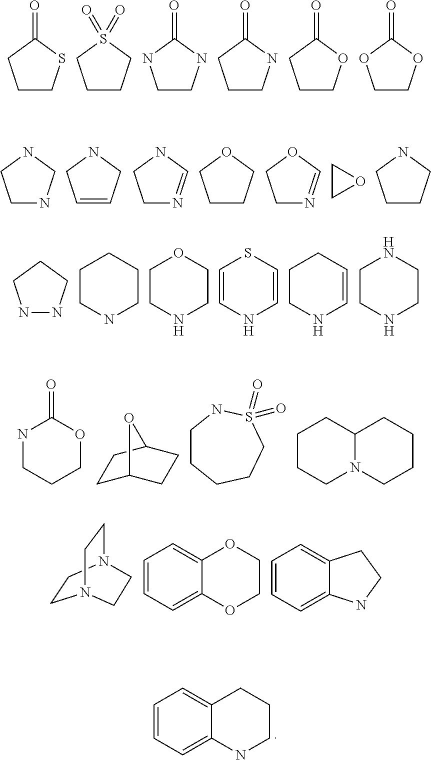

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₃)alkyl” means a functional group wherein a one- to three-carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heteroaryl” or “heteroaromatic” refers to a heterocycle having aromatic character. A polycyclic heteroaryl may include one or more rings that are partially saturated. Examples include the following moieties:

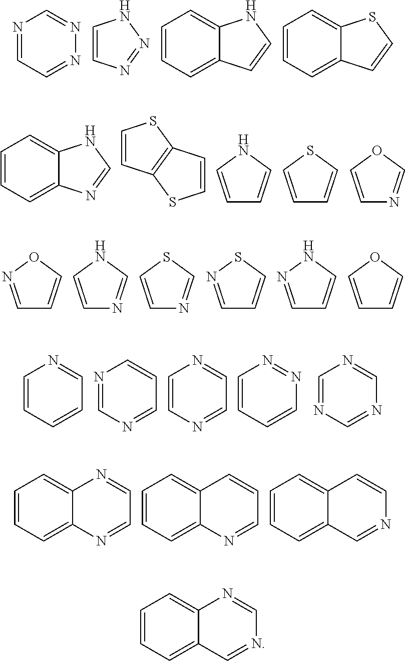

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

The compounds of the present invention may be synthesized using techniques well-known in the art of organic synthesis. The starting materials and intermediates required for the synthesis may be obtained from commercial sources or synthesized according to methods known to those skilled in the art.

In one aspect, the compound of the invention is a compound having the structure of Formula (I)

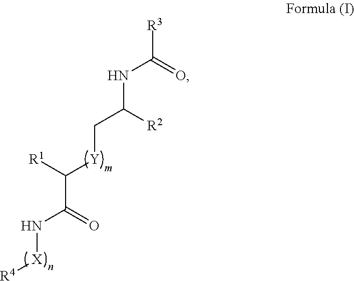

or a racemate, an enantiomer, a diastereomer, a salt, a pharmaceutically acceptable salt, or a derivative thereof.

In some embodiments, R₁ is selected from hydrogen, halogen, alkyl, substituted alkyl, cycloalkyl, substituted cyclocalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, heterocyclyl, substituted heterocyclyl, amino, aminoacetate, acyl, hydroxyl, alkoxy, carboxyl, carboxylate, ester, sulfoxy, or any combination thereof. In some embodiments, R₁ is selected from C₁-C₆ alkyl, substituted C₁-C₆ alkyl, aryl, phenyl, substituted phenyl, heteroaryl, substituted heteroaryl, heterocyclyl, substituted heterocyclyl, —(C₁-C₆)alkyl-aryl, substituted —(C₁-C₆)alkyl-aryl, —(C₁-C₆)alkyl-phenyl, substituted —(C₁-C₆)alkyl-phenyl, —(C₁-C₆)alkyl-carbocyclic, —(C₁-C₆)alkyl-heteroaryl, substituted —(C₁-C₆)alkyl-heteroaryl, N(R₅)(R₆), or any combination thereof. For example, in one embodiment, R₁ is —N(R₅)(R₆), —NH-isopropyl, —NH-cyclopentyl, —NH-phenyl, benzyl, —N(R₅)-benzyl, —CH₂-benzyl, —CH₂-napthyl, or any combination thereof.

In some embodiments, R₂ is selected from hydrogen, halogen, alkyl, substituted alkyl, cycloalkyl, substituted cyclocalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, heterocyclyl, substituted heterocyclyl, amino, aminoacetate, acyl, hydroxyl, alkoxy, carboxyl, carboxylate, ester, sulfoxy, or any combination thereof. In some embodiments, R₂ is selected from C₁-C₆ alkyl, substituted C₁-C₆ alkyl, aryl, phenyl, substituted phenyl, heteroaryl, substituted heteroaryl, heterocyclyl, substituted heterocyclyl, —(C₁-C₆)alkyl-aryl, substituted —(C₁-C₆)alkyl-aryl, —(C₁-C₆)alkyl-phenyl, substituted —(C₁-C₆)alkyl-phenyl, —(C₁-C₆)alkyl-carbocyclic, —(C₁-C₆)alkyl-heteroaryl, substituted —(C₁-C₆)alkyl-heteroaryl, N(R₅)(R₆), or any combination thereof. For example, in some embodiments, R₂ is selected from hydrogen, —(C₁-C₆)alkyl-carbocyclic, —(C₁-C₆)alkyl-phenyl, —(C₁-C₆)alkyl-substituted phenyl, —(C₁-C₆)alkyl-heteroaryl, or —(C₁-C₆)alkyl-substituted heteroaryl. In some embodiments, R₂ is selected from benzyl, —(C₁-C₆)alkyl-naphthyl, or —(C₁-C₆)alkyl-indolyl. In one embodiment, R₂ is benzyl. In one embodiment, R₂ is —CH₂-naphthyl. In one embodiment, R₂ is —CH₂-indolyl. In one embodiment, R₂ is biphenyl. In one embodiment, R₂ is fluorene.

In some embodiments, R₃ is selected from hydrogen, halogen, alkyl, substituted alkyl, cycloalkyl, substituted cyclocalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, heterocyclyl, substituted heterocyclyl, amino, aminoacetate, acyl, hydroxyl, alkoxy, carboxyl, carboxylate, ester, sulfoxy, or any combination thereof. In some embodiments, R₃ is selected from heteroaryl, substituted heteroaryl, heterocyclyl, substituted heterocyclyl, —N(R₅)(R₆), —(C₁-C₆)alkyl-NHC(═O)R₇, or any combination thereof. For example, in one embodiment, R₃ is furan. In one embodiment, R₃ is thiophene. In one embodiment, R₃ is benzofuran. In one embodiment, R₃ is benzothiophene. In one embodiment, R₃ is pyridine. In one embodiment, R₃ is isopropylthiazole. In one embodiment, R₃ is trifluoromethyl. In one embodiment, R₃ is benzylsulfonyl. In one embodiment, R₃ is 2,2,2-trifluoroacetamidyl. In one embodiment, R₃ is 2-furamidyl. In one embodiment, R₃ is nicotinamidyl.

In some embodiments, R₄ is selected from hydrogen, halogen, alkyl, substituted alkyl, cycloalkyl, substituted cyclocalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, heterocyclyl, substituted heterocyclyl, amino, aminoacetate, acyl, hydroxyl, alkoxy, carboxyl, carboxylate, ester, sulfoxy, or any combination thereof. In some embodiments, R₄ is selected from heteroaryl, substituted heteroaryl, heterocyclyl, substituted heterocyclyl, or any combination thereof. For example, in one embodiment, R₄ is pyridine. In one embodiment, R₄ is thiazole. In one embodiment, R₄ is imidazole. In one embodiment, R₄ is oxazole. In one embodiment, R₄ is pyrimidine. In one embodiment, R₄ is pyrazine. In one embodiment, R₄ is quinazoline. In one embodiment, R₄ is pyrrole.

In some embodiments, each occurrence of Y is independently selected from C(R₅)(R₆), N(R₅), S, O, or any combination thereof. In some embodiments, each occurrence of Y is independently selected from CH₂, NH, S, O, or any combination thereof. For example, in one embodiment, Y is selected from the group consisting of NH, S, and O. In one embodiment, Y is S.

In some embodiments, each occurrence of X is independently selected from alkyl, substituted alkyl, cycloalkyl, substituted cyclocalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, heterocyclyl, substituted heterocyclyl, amino, aminoacetate, acyl, hydroxyl, alkoxy, carboxyl, carboxylate, ester, sulfoxy, thiol, or any combination thereof. In some embodiments, each occurrence of X is independently selected from —(C₁-C₆) alkyl, —(C₁-C₆) alkyl-O—(C₁-C₆) alkyl-, or any combination thereof. For example, in one embodiment, X is C₁-C₆ alkyl. In one embodiment, X is C₃ alkyl. In one embodiment, X is alkyl-S—(C₁-C₆) alkyl-. In one embodiment, X is alkyl-NH—(C₁-C₆) alkyl-.

In some embodiments, each occurrence of R₅ is independently selected from hydrogen, halogen, alkyl, substituted alkyl, cycloalkyl, substituted cyclocalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, heterocyclyl, substituted heterocyclyl, amino, aminoacetate, acyl, hydroxyl, alkoxy, carboxyl, carboxylate, ester, sulfoxy, or any combination thereof. In some embodiments, each occurrence of R₅ is independently selected from hydrogen, C₁-C₆ alkyl, substituted C₁-C₆ alkyl, aryl, phenyl, substituted phenyl, heteroaryl, substituted heteroaryl, heterocyclyl, substituted heterocyclyl, —(C₁-C₆)alkyl-aryl, substituted —(C₁-C₆)alkyl-aryl, —(C₁-C₆)alkyl-phenyl, substituted —(C₁-C₆)alkyl-phenyl, —(C₁-C₆)alkyl-carbocyclic, —(C₁-C₆)alkyl-heteroaryl, substituted-(C₁-C₆)alkyl-heteroaryl, or any combination thereof.

In some embodiments, each occurrence of R₆ is independently selected from hydrogen, halogen, alkyl, substituted alkyl, cycloalkyl, substituted cyclocalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, heterocyclyl, substituted heterocyclyl, amino, aminoacetate, acyl, hydroxyl, alkoxy, carboxyl, carboxylate, ester, sulfoxy, or any combination thereof. In some embodiments, each occurrence of R₆ is independently selected from hydrogen, C₁-C₆ alkyl, substituted C₁-C₆ alkyl, aryl, phenyl, substituted phenyl, heteroaryl, substituted heteroaryl, heterocyclyl, substituted heterocyclyl, —(C₁-C₆)alkyl-aryl, substituted —(C₁-C₆)alkyl-aryl, —(C₁-C₆)alkyl-phenyl, substituted —(C₁-C₆)alkyl-phenyl, —(C₁-C₆)alkyl-carbocyclic, —(C₁-C₆)alkyl-heteroaryl, substituted-(C₁-C₆)alkyl-heteroaryl, or any combination thereof.

In some embodiments, each occurrence of R₇ is independently selected from hydrogen, halogen, alkyl, substituted alkyl, cycloalkyl, substituted cyclocalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, heterocyclyl, substituted heterocyclyl, amino, aminoacetate, acyl, hydroxyl, alkoxy, carboxyl, carboxylate, ester, sulfoxy, or any combination thereof. In some embodiments, each occurrence of R₇ is independently selected from phenyl, substituted phenyl, heteroaryl, substituted heteroaryl, heterocyclyl, substituted heterocyclyl, and any combination thereof. For example, in one embodiment, R₇ is furan. In one embodiment, R₇ is thiophene. In one embodiment, R₇ is benzofuran. In one embodiment, R₇ is benzothiophene. In one embodiment, R₇ is pyridine.

In some embodiments, m is an integer from 0 to 10. In some embodiments, m is an integer from 0 to 5. In some embodiments, m is an integer from 0 to 3. In some embodiments, m is an integer from 0 to 2. In some embodiments, m is an integer from 0 to 1. For example, in one embodiment, m is an integer 10. In one embodiment, m is an integer 9. In one embodiment, m is an integer 8. In one embodiment, m is an integer 7. In one embodiment, m is an integer 6. In one embodiment, m is an integer 5. In one embodiment, m is an integer 4. In one embodiment, m is an integer 3. In one embodiment, m is an integer 2. In one embodiment, m is an integer 1. In one embodiment, m is an integer 0.

In some embodiments, n is an integer from 0 to 10. In some embodiments, n is an integer from 0 to 5. In some embodiments, n is an integer from 0 to 3. In some embodiments, n is an integer from 0 to 2. In some embodiments, n is an integer from 0 to 1. For example, in one embodiment, n is an integer 10. In one embodiment, n is an integer 9. In one embodiment, n is an integer 8. In one embodiment, n is an integer 7. In one embodiment, n is an integer 6. In one embodiment, n is an integer 5. In one embodiment, n is an integer 4. In one embodiment, n is an integer 3. In one embodiment, n is an integer 2. In one embodiment, n is an integer 1. In one embodiment, n is an integer 0.

In various embodiments, the compound having the structure of Formula (I) is at least one compound selected from.

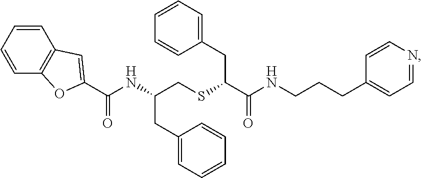 [SMILES: O=C(N[C@H](CS[C@H](Cc1ccccc1)C(=O)NCCCc1ccncc1)Cc1ccccc1)c1cc2ccccc2o1]

or a racemate, an enantiomer, a diastereomer, a salt, a pharmaceutically acceptable salt, or a derivative thereof,

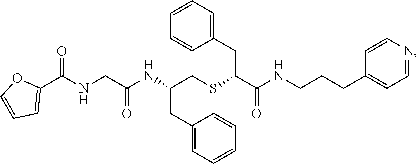 [SMILES: O=C(CNC(=O)c1ccco1)N[C@H](CS[C@H](Cc1ccccc1)C(=O)NCCCc1ccncc1)Cc1ccccc1]

or a racemate, an enantiomer, a diastereomer, a salt, a pharmaceutically acceptable salt, or a derivative thereof,

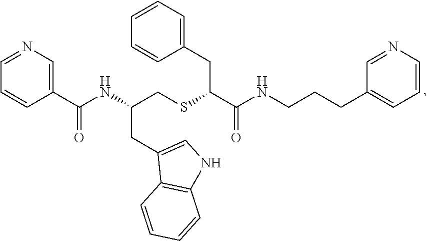 [SMILES: O=C(N[C@H](CS[C@H](Cc1ccccc1)C(=O)NCCCc1cccnc1)Cc1c[nH]c2ccccc12)c1cccnc1]

or a racemate, an enantiomer, a diastereomer, a salt, a pharmaceutically acceptable salt, or a derivative thereof,

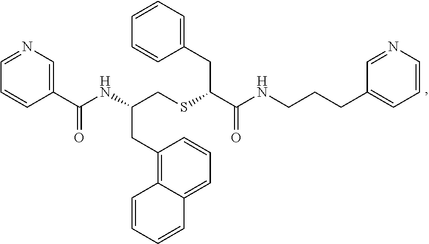 [SMILES: O=C(N[C@H](CS[C@H](Cc1ccccc1)C(=O)NCCCc1cccnc1)Cc1cccc2ccccc12)c1cccnc1]

or a racemate, an enantiomer, a diastereomer a salt, a pharmaceutically acceptable salt, or a derivative thereof,

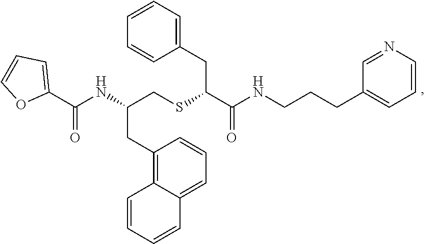 [SMILES: O=C(N[C@H](CS[C@H](Cc1ccccc1)C(=O)NCCCc1cccnc1)Cc1cccc2ccccc12)c1ccco1]

or a racemate, an enantiomer, a diastereomer a salt a pharmaceutically acceptable salt, or a derivative thereof, or

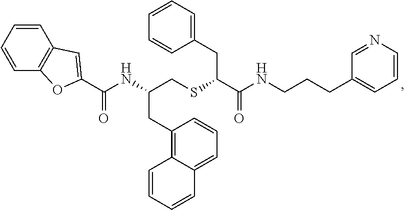 [SMILES: O=C(N[C@H](CS[C@H](Cc1ccccc1)C(=O)NCCCc1cccnc1)Cc1cccc2ccccc12)c1cc2ccccc2o1]

or a racemate, an enantiomer, a diastereomer, a salt, a pharmaceutically acceptable salt, or a derivative thereof.

### Preparation of the Compounds of the Invention

Compounds having the structure of Formula (I) may be prepared by the general schemes described herein, using the synthetic method known by those skilled in the art. The following examples illustrate non-limiting embodiments of the invention.

In a non-limiting embodiment, a primary alcohol is protected with a leaving group, such as a tosyl group, forming compound A. The tosyl group is then displaced by B in the presence of a base to yield carboxylic acid C. C is then treated with an amine D, 1-ethyl-3-(3-dimethylaminopropyl)carbodiimide (EDAC) and a base such as diisoproyplyethylamine (DIPEA) to produce amide E. The ester group is cleaved from amide E using trifluoroacetic acid (TFA) and a base, yielding primary amine F. Primary amine F is reacted with alcohol G in the presence of EDAC and a base such as DIPEA to produce compounds of Formula (I).

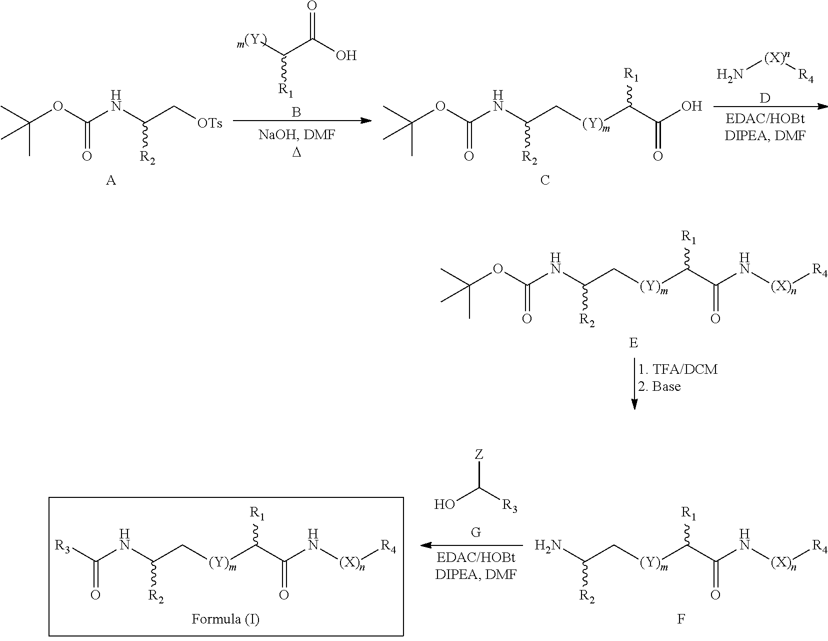

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention may exist as tautomers. All tautomers are included within the scope of the compounds presented herein.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In one embodiment, sites on, for example, the aromatic ring portion of compounds of the invention are susceptible to various metabolic reactions. Incorporation of appropriate substituents on the aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference in their entirety). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

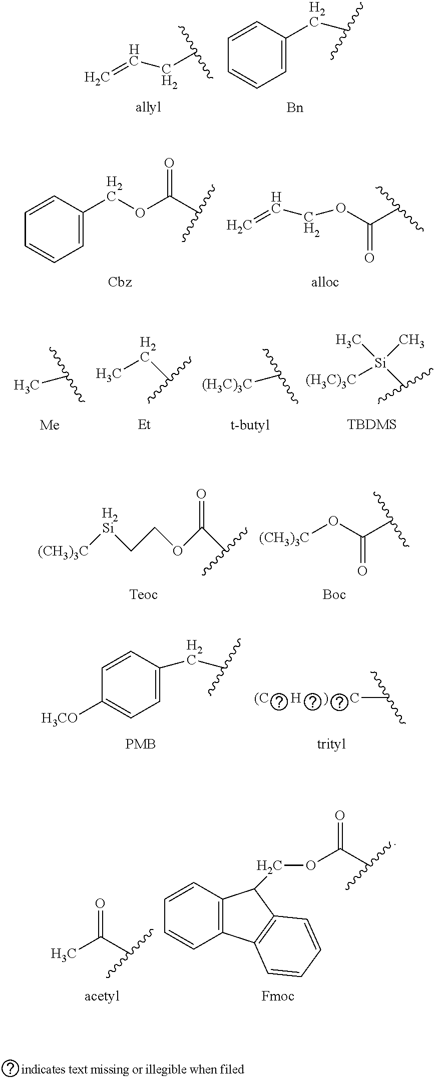

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Methods of the Invention

In one aspect, the invention includes a method of inhibiting CYP3A4 in a subject in need thereof. In another aspect, the present invention provides a method of treating or preventing at least one disease or disorder associated with CYP3A4 in a subject in need thereof. The method comprises administering to the subject an effective amount of a therapeutic composition comprising a compound of the invention. The method further comprises administering to the subject an effective amount of a therapeutic composition comprising a compound of the invention for the treatment of HIV. In one embodiment, the method further comprises administering to the subject an additional therapeutic agent.

In one embodiment, the compound of the invention and the therapeutic agent are co-administered to the subject. In another embodiment, the compound of the invention and the therapeutic agent are coformulated and co-administered to the subject.

In some embodiments, the therapeutic agent is an antiviral agent, anti-cancer agent, immunosuppressant agent, or any combination thereof.

In one embodiment, administering the compound of the invention to the subject allows for administering a lower dose of the therapeutic agent compared to the dose of the therapeutic agent alone that is required to achieve similar results in treating the subject. For example, in one embodiment, the compound of the invention inhibits metabolism of the additional therapeutic compound, thereby allowing for a lower dose of the therapeutic compound to provide the same effect. In another embodiment, the compound of the invention inhibits metabolism of the HIV therapeutic compounds and thereby act as a pharmacoenhancer.

In one embodiment, the subject is a mammal. In another embodiment, the mammal is a human.

### Combination Therapies

The compounds of the present invention may be useful in combination with one or more additional compounds. In certain embodiments, these additional compounds may comprise compounds of the present invention or therapeutic agents which are known antivirals. In certain embodiments, the antiviral agent may comprise compounds useful for treating HIV infections. Such compounds include, but are not limited to, compounds which are known to treat, prevent, or reduce the symptoms of HIV infections

In non-limiting examples, the compounds useful within the invention may be used in combination with one or more of the following anti-HIV drugs: HIV Combination Drugs: efavirenz, emtricitabine or tenofovir disoproxil fumarate (Atripla®/BMS, Gilead); lamivudine or zidovudine (Combivir®/GSK); abacavir or lamivudine (Epzicom®/GSK); abacavir, lamivudine or zidovudine (Trizivir®/GSK); emtricitabine, tenofovir disoproxil fumarate (Truvada®/Gilead).

Entry and Fusion Inhibitors: maraviroc (Celsentri®, Selzentry®/Pfizer); pentafuside or enfuvirtide (Fuzeon®/Roche, Trimeris).

Integrase Inhibitors: raltegravir or MK-0518 (Isentress®/Merck).

Non-Nucleoside Reverse Transcriptase Inhibitors: delavirdine mesylate or delavirdine (Rescriptor®/Pfizer); nevirapine (Viramune®/Boehringer Ingelheim); stocrin or efavirenz (Sustiva®/BMS); etravirine (Intelence®/Tibotec).

Nucleoside Reverse Transcriptase Inhibitors: lamivudine or 3TC (Epivir®/GSK); FTC, emtricitabina or coviracil (Emtriva®/Gilead); abacavir (Ziagen®/GSK); zidovudina, ZDV, azidothymidine or AZT (Retrovir®/GSK); ddI, dideoxyinosine or didanosine (Videx®/BMS); abacavir sulfate plus lamivudine (Epzicom®/GSK); stavudine, d4T, or estavudina (Zerit®/BMS); tenofovir, PMPA prodrug, or tenofovir disoproxil fumarate (Viread®/Gilead).

Protease Inhibitors: amprenavir (Agenerase®/GSK, Vertex); atazanavir (Reyataz®/BMS); tipranavir (Aptivus®/Boehringer Ingelheim); darunavir (Prezist®/Tibotec); fosamprenavir (Telzir®, Lexiva®/GSK, Vertex); indinavir sulfate (Crixivan®/Merck); saquinavir mesylate (Invirase®/Roche); lopinavir or ritonavir (Kaletra®/Abbott); nelfinavir mesylate (Viracept®/Pfizer); ritonavir (Norvir®/Abbott).

A synergistic effect may be calculated, for example, using suitable methods such as, for example, the Sigmoid-Eₘₐₓ equation (Holford & Schemer, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either before or after the onset of a disease or infection. Further, several divided dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, (e.g., human), may be carried out using known procedures, at dosages and for periods of time effective to treat the disease or infection in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or infection in the patient. Dosage regimens may be adjusted to provide the optimum therapeutic response. For example, several divided doses may be administered daily. In another example, the dose may be proportionally reduced as indicated by the exigencies of the therapeutic situation. A non-limiting example of an effective dose range for a therapeutic compound of the invention is from about 1 mg/kg to about 5,000 mg/kg of body weight/per day. One of ordinary skill in the art would be able to assess the relevant factors and make the determination regarding the effective amount of the therapeutic compound without undue experimentation.

Actual dosage levels of the active ingredients in the pharmaceutical compositions of this invention may be varied to obtain an amount of the active ingredient that is effective to achieve the desired therapeutic response for a particular patient, composition, and mode of administration, without generating excessive side effects in the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical professional, e.g., physician or veterinarian, having ordinary skill in the art may readily determine and prescribe the effective amount of the pharmaceutical composition required. For example, the physician or veterinarian could start with a dosage of the compound of the invention in the pharmaceutical composition at a level that is lower than the level required to achieve the desired therapeutic effect, and then increase the dosage over time until the desired effect is achieved.

In particular embodiments, it is advantageous to formulate the compound in dosage unit form for ease of administration and uniformity of dosage. “Dosage unit form” as used herein refers to a physically discrete unit containing a predetermined quantity of therapeutic compound calculated to produce the desired therapeutic effect, in association with the required pharmaceutical vehicle. The dosage unit forms of the invention can be selected based upon (a) the unique characteristics of the therapeutic compound and the particular therapeutic effect to be achieved, and (b) the limitations inherent in the art of compounding/formulating such a therapeutic compound for the treatment of a disease or infection in a patient.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), vegetable oils, and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, it is useful to include isotonic agents, for example, sugars, sodium chloride, or polyalcohols such as mannitol and sorbitol, in the composition. Prolonged absorption of the injectable compositions can be achieved by including in the composition an agent which delays absorption, for example, aluminum monostearate or gelatin. In one embodiment, the pharmaceutically acceptable carrier is DMSO, alone or in combination with other carriers.

The therapeutically effective amount or dose of a compound of the present invention depends on the age, sex and weight of the patient, the current medical condition of the patient and the severity of the disease or infection in the patient being treated. The skilled artisan is able to determine appropriate doses depending on these and other factors.

The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Doses of the compound of the invention for administration may be in the range of from about 1 μg to about 10,000 mg, from about 20 μg to about 9,500 mg, from about 40 μg to about 9,000 mg, from about 75 μg to about 8,500 mg, from about 150 μg to about 7,500 mg, from about 200 μg to about 7,000 mg, from about 3050 μg to about 6,000 mg, from about 500 μg to about 5,000 mg, from about 750 μg to about 4,000 mg, from about 1 mg to about 3,000 mg, from about 10 mg to about 2,500 mg, from about 20 mg to about 2,000 mg, from about 25 mg to about 1,500 mg, from about 30 mg to about 1,000 mg, from about 40 mg to about 900 mg, from about 50 mg to about 800 mg, from about 60 mg to about 750 mg, from about 70 mg to about 600 mg, from about 80 mg to about 500 mg, and any and all whole or partial increments therebetween.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, the dosage of a second compound as described elsewhere herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are administered to the patient from about one to about five times per day or more. In various embodiments, the compositions of the invention are administered to the patient, 1-7 times per day, 1-7 times every two days, 1-7 times every 3 days, 1-7 times every week, 1-7 times every two weeks, and 1-7 times per month. It is readily apparent to one skilled in the art that the frequency of administration of the various combination compositions of the invention will vary from individual to individual depending on many factors including, but not limited to, age, the disease or disorder to be treated, the severity of the disease or disorder to be treated, gender, overall health, and other factors. Thus, the invention should not be construed to be limited to any particular dosing regime and the precise dosage and composition to be administered to any patient is determined by the medical professional taking all other factors about the patient into account.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, may be reduced to a level at which the improved disease is retained. In some embodiments, a patient may require intermittent treatment on a long-term basis, or upon any recurrence of the disease or disorder.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the present invention is directed to a packaged pharmaceutical composition comprising a container holding a therapeutically effective amount of a compound of the invention, alone or in combination with a second pharmaceutical agent; and instructions for using the compound to treat or prevent a disease or infection in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, suitable forms include tablets, dragees, liquids, drops, suppositories, or capsules, caplets and gelcaps. The compositions formulated for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation involves the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of G-protein receptor-related diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release refers to a drug formulation that provides for gradual release of a drug over an extended period of time, and that may, although not necessarily, result in substantially constant blood levels of a drug over an extended time period. The period of time may be as long as a day, a week, or a month or more and should be a release which is longer that the same amount of agent administered in bolus form. The term delayed release is used herein in its conventional sense to refer to a drug formulation that provides for an initial release of the drug after some delay following drug administration and that mat, although not necessarily, includes a delay of from about 10 minutes up to about 12 hours.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term pulsatile release refers to a drug formulation that provides release of the drug in such a way as to produce pulsed plasma profiles of the drug after drug administration.

The term immediate release refers to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art recognize, or are able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: High Potency Inhibitors of Human CYP3A4

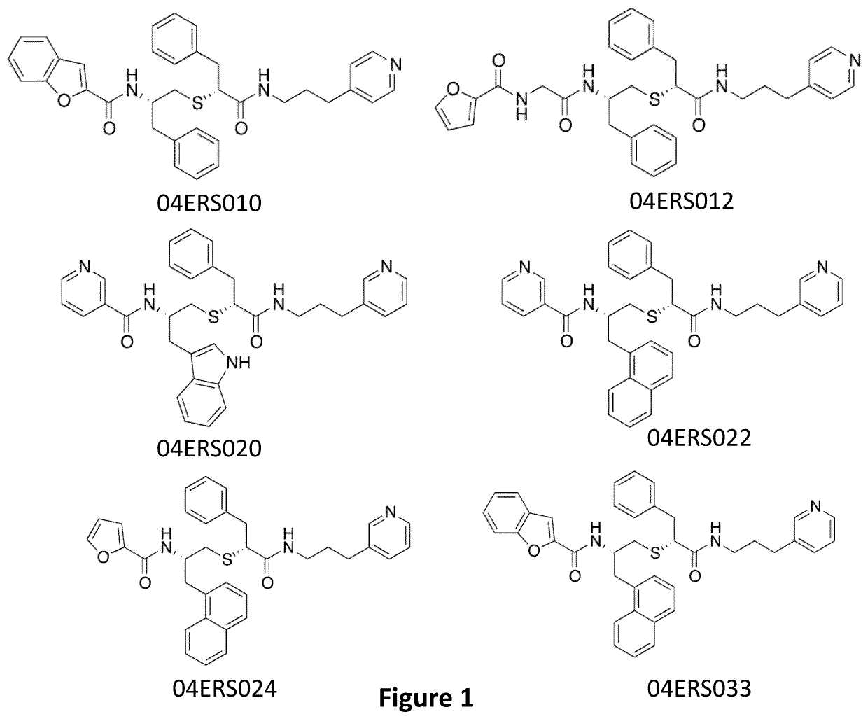

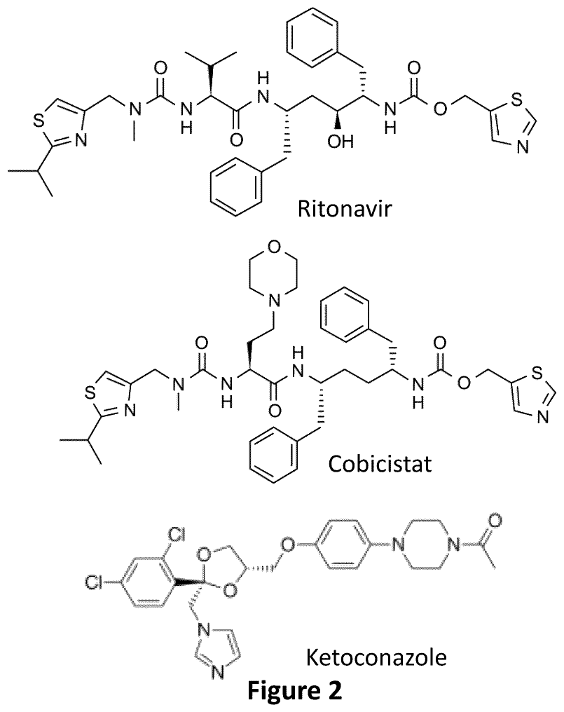

Based on the structure/function studies on the interaction of CYP3A4 with ritonavir-like molecules, the inhibitory mechanism of these molecules was elucidated. In addition, how each functional group contributes to the binding affinity and inhibitory potency for CYP3A4 was determined. Compounds with improved backbone spacing, side, and terminal group combinations were synthesized (FIG. 1) to yield series III and IV compounds that had comparable or higher affinity and inhibitory potency for CYP3A4. Importantly, these compounds were also easier and less expensive to synthesize than ritonavir and cobicistat (FIG. 2). These new CYP3A4 inhibitors could be used as pharmacoenhancers at lower, less toxic doses to slow down metabolism of HIV protease inhibitors, immunosuppressants, anti-cancer drugs, and many other pharmaceuticals that are quickly biotransformed and cleared by CYP3A4. Additionally, the undemanding chemical synthesis could significantly lower the production and treatment costs.

The inventive compounds are unique because they were designed based on the CYP3A4-inhibitor co-crystal structures. Comparative analysis of the ligand-induced spectral perturbations, binding affinity, heme ligation kinetics, inhibitor potency, and association mode of the analogs enabled the development of highly potent CYP3A4 inhibitors that are superior to the currently available pharmacoenhancers in several ways. Compared to ritonavir and cobicistat, the inventive compounds bind to and inhibit CYP3A4 several-fold stronger (Table 1), have a smaller size (<600 versus 72-776 Daltons), are more soluble (lipophilicity index log P<6 versus 7.5), and are easier to produce. Synthesis of ritonavir and cobicistat is a complicated multi-step process that requires harsh reagents and/or expensive catalysts. The inventive compounds can be produced with moderate to high yields using the scheme disclosed elsewhere herein. Since the inventive compounds are expected to have both higher availability and potency in vivo, they can be administered at lower does to achieve a pharmacoenhancing effect, which should further lower treatment costs.

TABLE 1
K_{d} and IC₅₀ of inventive compounds
relative to ritonavir and cobicistat.

| | K_{d} (nM) | IC₅₀ (nM) |
|---|---|---|
| 04ERS010 | 7 | 170 |
| 04ERS012 | 10 | 150 |
| 04ERS020 | 16 | 220 |
| 04ERS022 | 2 | 90 |
| 04ERS024 | 3 | 150 |
| 04ERS033 | 4 | 200 |
| Ritonavir | 15 | 300 |
| Corbicistat | 55 | 600 |

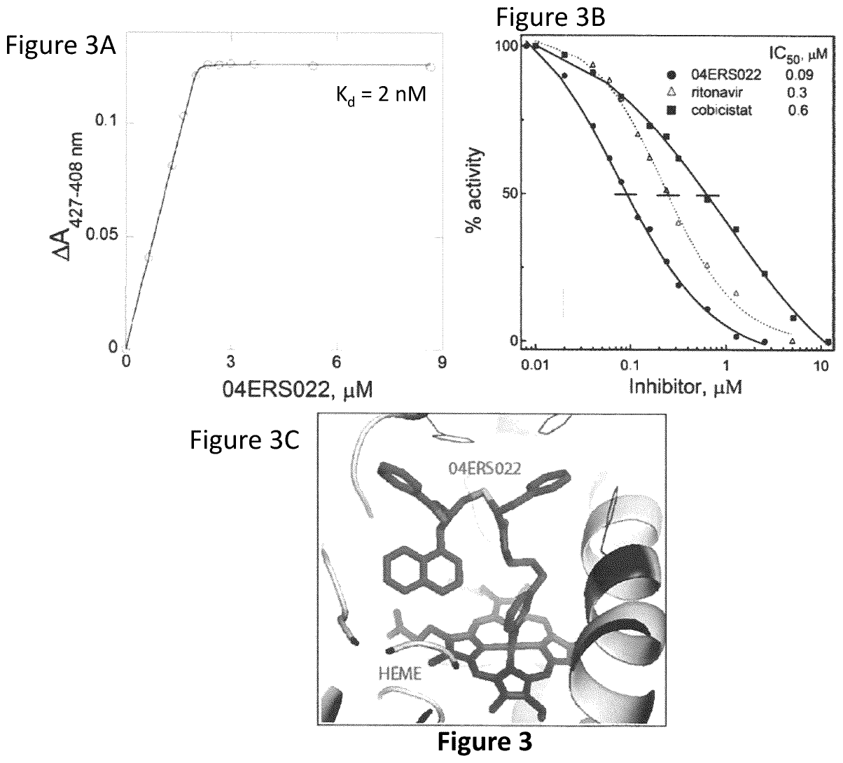

Spectral K_{d} was calculated from the equilibrium titration plots (FIG. 3A). Functional assays were conducted in a reconstituted system with cytochrome P450 reductase and 7-benzyloxy-4-(trifluoromethyl) coumarin (BFC) as a substrate, and IC₅₀ was derived from the % activity versus inhibitor concentration plots (FIG. 3B).

Inventive CYP3A4 inhibitors (FIG. 1) may include a pyridine ring with the nitrogen in a para- or meta-position which serves as the heme-ligating moiety (R₄ in Formula (I)). Although not wishing to be bound by any particular theory, it is hypothesized that the flexible backbone helps the inhibitor to properly orient in the CYP3A4 active site and to maximize hydrophobic interactions via the phenyl and/or naphthalene rings (R₁ and R₂ functional groups in Formula (I)). Moreover, terminal nicotinamide, furan, benzofuran, or furoylglycine moieties were found to interact with the F-G peptide and stabilize the protein-inhibitor complex. As an example, the binding mode of the heme-ligated 04ERS022 in the co-crystal structure with CYP3A4 is shown in FIG. 3C.

### Example 2: Rational Design of CYP3A4 Inhibitors: A One-Atom Linker Elongation in Ritonavir-Like Compounds LED to a Marked Improvement in the Binding Strength

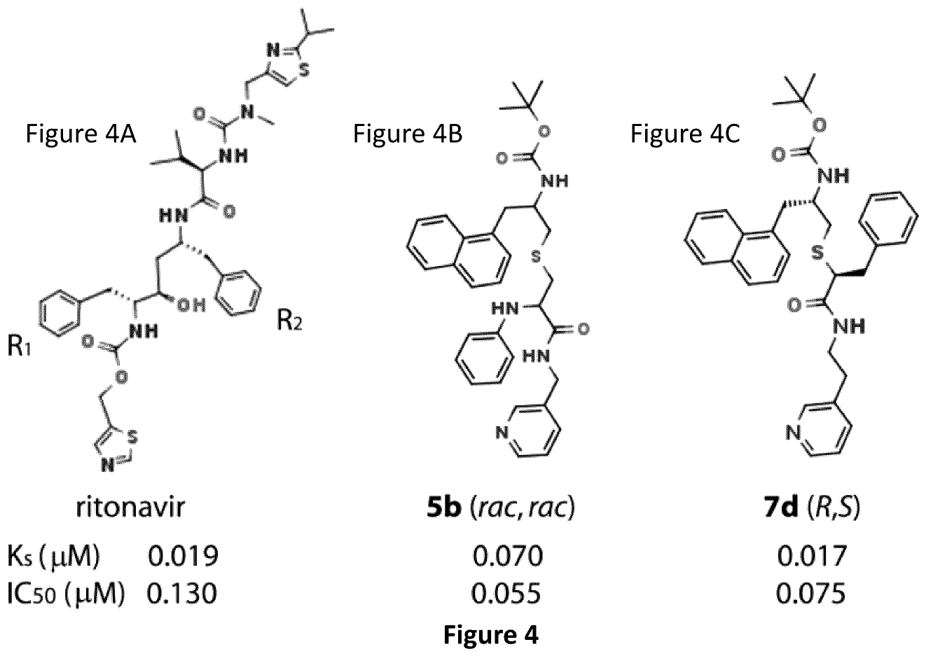

Cytochrome P450 (CYP) enzymes are monooxygenases that mediate xenobiotic metabolism and synthesis of steroids, vitamins and fatty acids (Manikandan P et al., 2018, Curr. Drug Targets, 19:38-54; Rendic S P et al., 2018, Drug Metab. Rev., 50:256-342). In humans, CYP3A4 is the major liver and intestinal P450 isoform with a large and malleable active site which can accommodate chemically diverse compounds. As a result, CYP3A4 clears the majority of administered drugs along with other foreign compounds, such as environmental pollutants, insecticides and pesticides (Guengerich F P et al., 1991, Chem. Res. Toxicol., 4:391-407; Li A P et al., 1995, Toxicology, 104:1-8; Mehmood Z et al., 1996, Chemosphere, 33:759-769). Drugs may serve not only as substrates but also as inducers and inhibitors of CYP3A4 (Zhou S F, 2008, Curr. Drug Metab., 9:310-322). Inhibition of CYP3A4 is usually undesired, because it could lead to drug toxicity, drug-drug interactions and other serious adverse effects. However, when carefully controlled, CYP3A4 inhibition can improve therapeutic efficacy of drugs by slowing down their conversion to more water-soluble, readily excreted metabolites. This beneficial pharmacoenhancing (boosting) effect is currently used for treatment of HIV and HCV infections, where potent CYP3A4 inhibitors, ritonavir (FIG. 4A) and its derivative cobicistat, are co-administered with anti-viral drugs that otherwise are quickly metabolized by CYP3A4 (Kempf D J et al., 1997, Antimicrob. Agents Chemother., 41:654-660; Xu L et al., 2010, ACS Med. Chem. Lett., 1:209-213). Since neither booster was developed based on the 3D-structure of CYP3A4, it remains unclear what structural determinants are needed for potent inhibition and whether the inhibitory potency could be further improved. In this regard, detailed studies on ritonavir-like compounds could help unravel the inhibitory mechanism and design more effective pharmacoenhancers.

Previous work on the analogues of desoxyritonavir (provided by Gilead Sciences) identified pyridine as the strongest heme-ligating group and helped develop a pharmacophore model for a potent CYP3A4 inhibitor (Sevrioukova I F et al., 2013, J. Med. Chem., 56:3733-3741; Sevrioukova I F et al., 2014, Curr. Top. Med. Chem., 14:1348-1355). Subsequent studies (Kaur P et al., 2016, J. Med. Chem., 59:4210-4220; Samuels E R et al., 2018, Mol. Pharm., 15:279-288; Samuels E R et al., 2019, Biochemistry, 58:2077-2087; Samuels E R et al., 2020, Bioorg. Med. Chem., 28:115349-115360) utilized a build-from-scratch approach to evaluate the importance of other pharmacophoric determinants, such as the backbone length/composition and the size/hydrophobicity of the R₁ and R₂ side-group substituents, represented by phenyls in ritonavir (FIG. 4A). It was found that the binding affinity and inhibitory potency of ritonavir-like compounds depend on the spacing between the functional groups, H-bonding to the active site S119, and the side-group hydrophobicity and stereochemistry. One particular observation was that a one-carbon linker extension, from pyridyl-methyl to pyridyl-ethyl, and/or phenyl>indole>naphthalene substitution in R₂ largely improve the binding affinity and IC₅₀ (Samuels E R et al., 2019, Biochemistry, 58:2077-2087; Samuels E R et al., 2020, Bioorg. Med. Chem., 28:115349-115360). The most potent inhibitors designed so far, compounds 5b and 7d (series IV; Samuels E R et al., 2020, Bioorg. Med. Chem., 28:115349-115360), are shown in FIG. 4B and FIG. 4C.

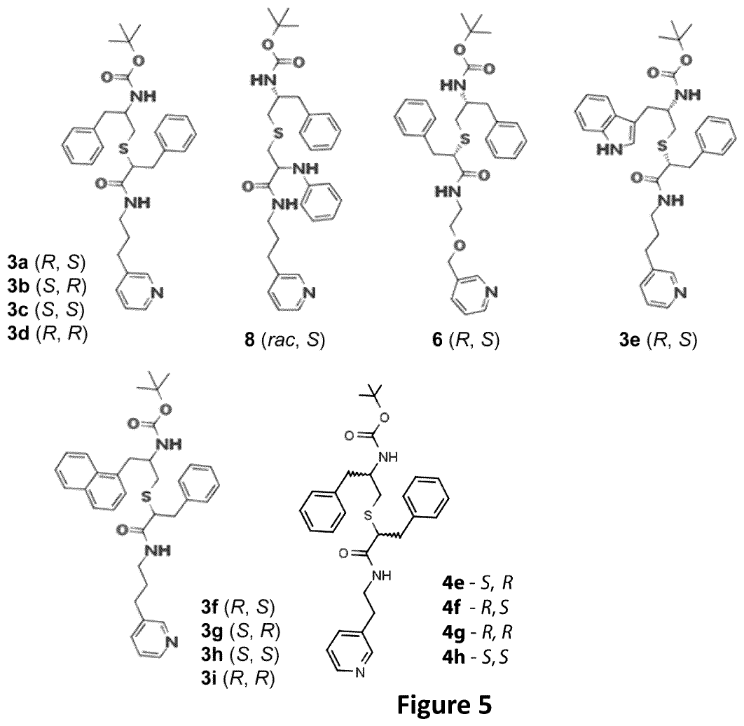

The current study was set to test the benefit of further pyridyl-linker extensions. For this purpose, eleven (series V) compounds were synthesized with the pyridyl-propyl or pyridyl-methoxyethyl (butyl-like) spacer, R₁-phenyl, and R₂-phenyl/indole/naphthalene in various stereo configurations (FIG. 5). It was found that one—but not two-atom linker extension led to tighter binding and more potent inhibition of CYP3A4. According to the structural data, the elongated compounds adopted a more relaxed conformation, which enabled stronger N-pyridine ligation to the heme. There was also a downside effect—an increase in ligand mobility in the active site. Therefore, based on structure-activity analysis of series IV and V analogues, it was concluded that further improvement in the inhibitory power are likely to be achieved by balancing flexibility/adjustability and rigidity of the scaffold.

Series V Analogues

While working on series III inhibitors (Samuels E R et al., 2019, Biochemistry, 58:2077-2087; the disclosure of which is hereby incorporated by reference in its entirety), it was found that a one-atom elongation of the spacer between the heme-ligating pyridine and the R₁ side-group, from five- to six-atom separation, improved the binding affinity and inhibitory potency for CYP3A4. Studies on series IV analogues (Samuels E R et al., 2020, Bioorg. Med. Chem., 28:115349-115360), in turn, showed that the size/hydrophobicity of R₂ was another factor critical for the binding and inhibitory strength, which could be modulated via R₁/R₂ stereochemistry. To further investigate structure-activity relationship (SAR) of ritonavir-like inhibitors, eleven (series V) analogues were designed to test the benefits of further extensions of the pyridyl-linker by one or two atoms. The first subseries, compounds 3a-d and 8, had a pyridyl-propyl linker and R₁/R₂-phenyls in different stereo configuration (FIG. 5). Compound 8 (rac, S) was included to test the effect of a longer R₁/R₂ spacer (five-vs. four-atom for other analogues). Compound 6 (R, S) also contained R₁/R₂-phenyls and an extended pyridyl-methoxyethyl (butyl-like) linker. The second pyridyl-propyl subseries was designed to assess the impact of a bulkier R₂, represented by indole in compound 3e (R, S) and naphthalene in compounds 3f-i in various stereo configurations. Compounds 6 and 3e were synthesized in the R/S configuration because it was more preferable for the binding to CYP3A4 (Samuels E R et al., 2020, Bioorg. Med. Chem., 28:115349-115360). As previously, spectral titrations, inhibitory and temperature denaturation assays, kinetic measurements, and co-crystallization of analogues with CYP3A4 were conducted for SAR evaluation.

3a-d Analogues

Spectral and Biochemical Properties of Compounds 3a-d

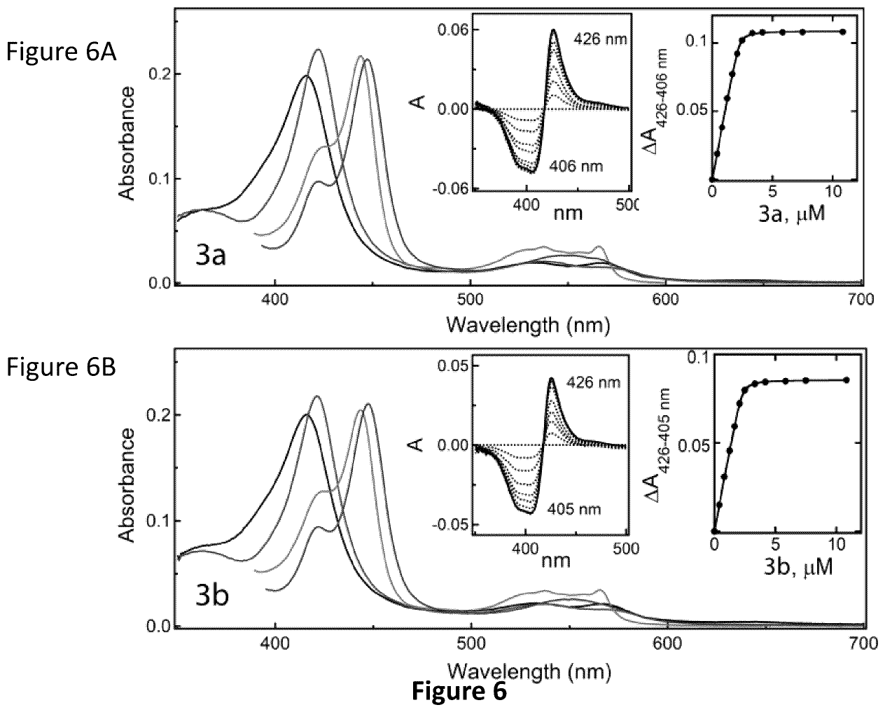

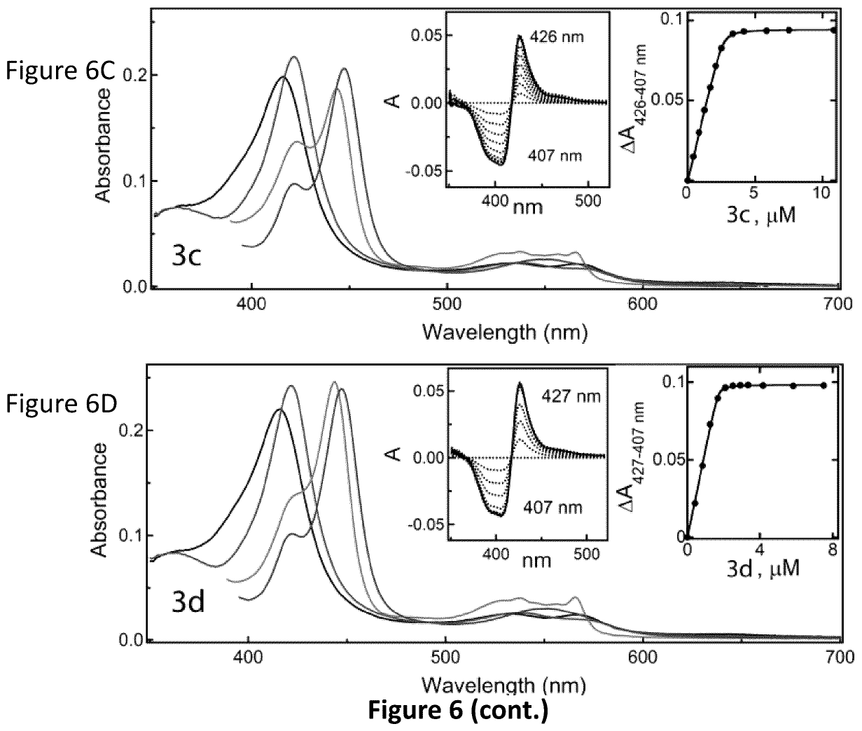

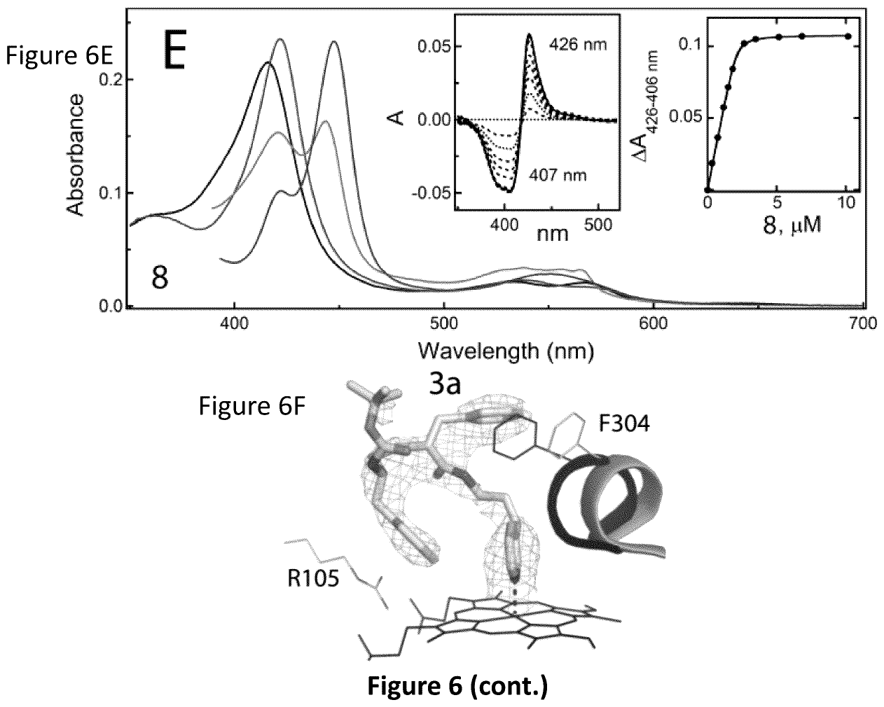

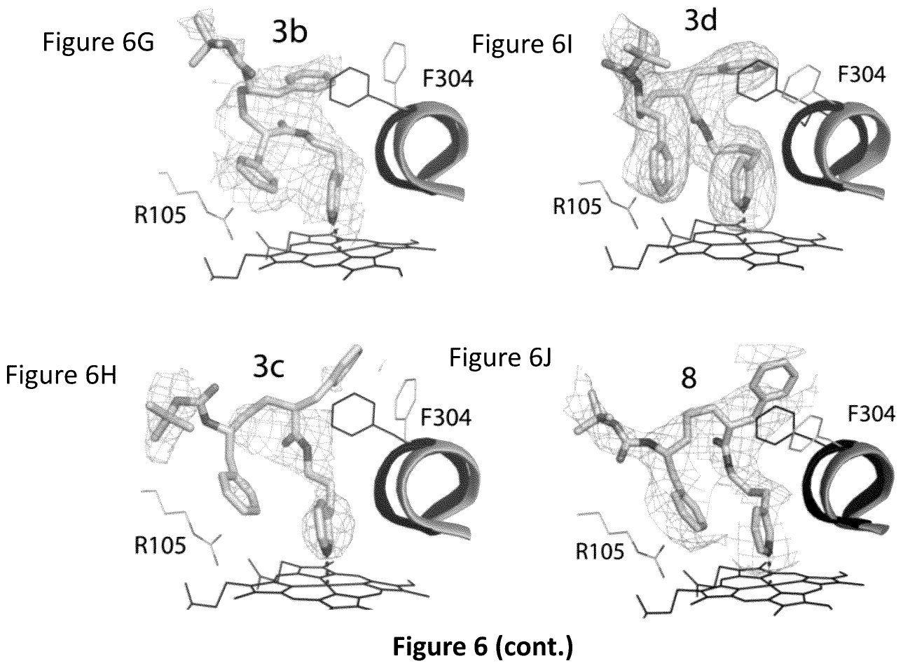

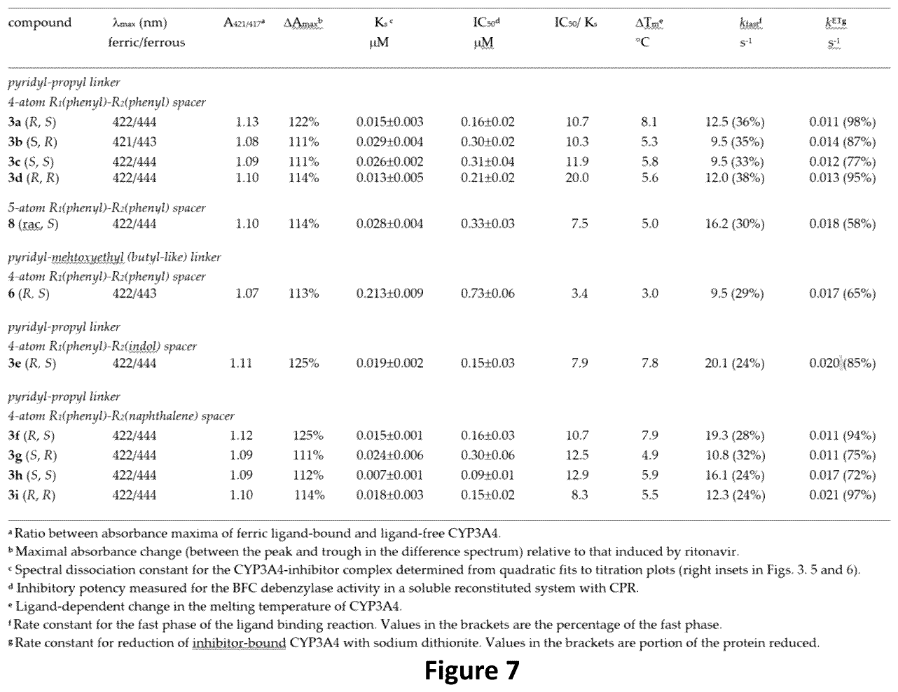

Compared to the pyridyl-ethyl containing compounds 4e-h (FIG. 5; series III analogues; Samuels E R et al., 2019, Biochemistry, 58:2077-2087; the disclosure of which is hereby incorporated by reference in its entirety), there was a marked improvement in the binding and inhibitory properties of compounds 3a-d. Except 3b (R, S), all compounds induced a larger red shift in the Soret band (type II spectral change; FIG. 6A through FIG. 6D), with λₘₐₓ for the ferric and ferrous CYP3A4 at 422 nm and 444 nm, respectively, vs. 421 nm and 443 nm for compounds 4e-h. Two other spectral parameters reflecting the strength of N-pyridine ligation, A_{417/422} nm (ratio between the absorbance maxima in the absolute ligand-free/bound spectra) and ΔAₘₐₓ (peak/trough amplitude in the difference spectra; left insets in FIG. 6A through FIG. 6D), were the highest observed so far (FIG. 7). Spectral dissociation constants (Kₛ, measure of the binding affinity) were derived from titration plots (right insets in FIG. 6A through FIG. 6D). Compared to compounds 4e-h, the binding affinity of compounds 3a-d was 2-to-3-fold higher: Kₛ of 0.013-0.029 μM vs. 0.040-0.055 μM, respectively. The IC₅₀ values for the BFC debenzylase activity of CYP3A4 correlated with Kₛ and decreased by 1.5-to-5-fold upon linker extension (FIG. 7). The lowest IC₅₀ was derived for compounds 3a (0.16 μM). Another notable improvement was in thermostability of ligand-bound CYP3A4, whose melting temperature (Tₘ) increased by 5.3-8.1° C. upon 3a-d ligation. Again, the highest ΔTₘ for observed for the CYP3A4-3a complex. For comparison, ΔTₘ for 4e-h did not exceed 4.7° C. (Samuels E R et al., 2019, Biochemistry, 58:2077-2087). The ligand binding rate (k_{fast} in FIG. 7) was not affected by linker modification and remained within the 9.5-12.5 s⁻¹ range.

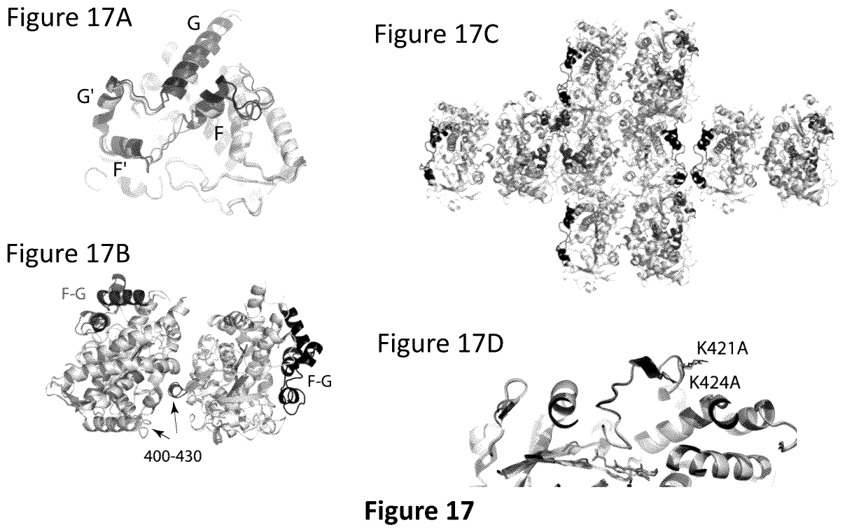

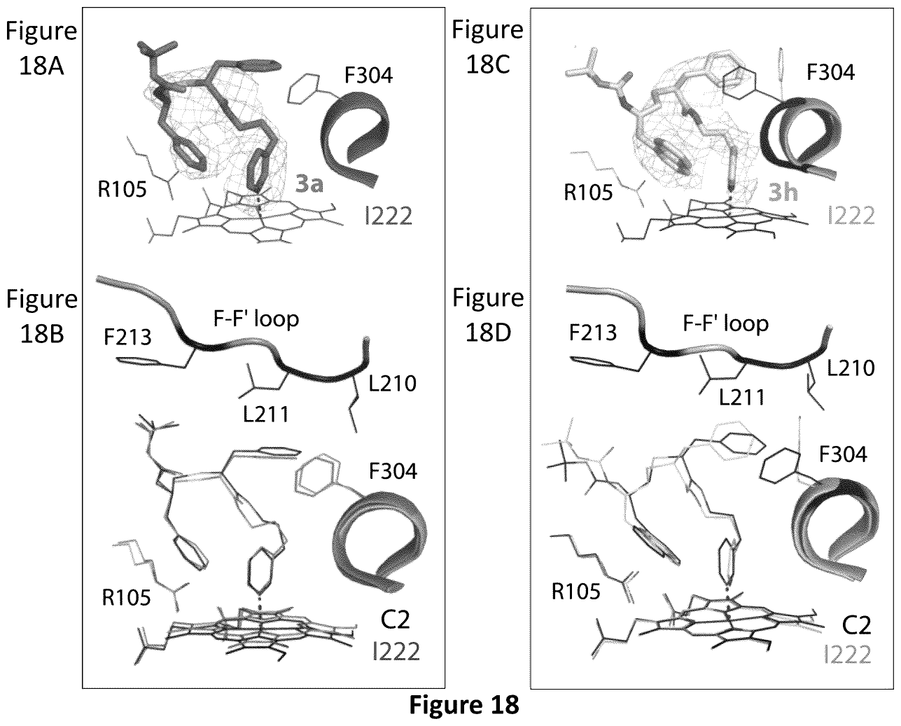

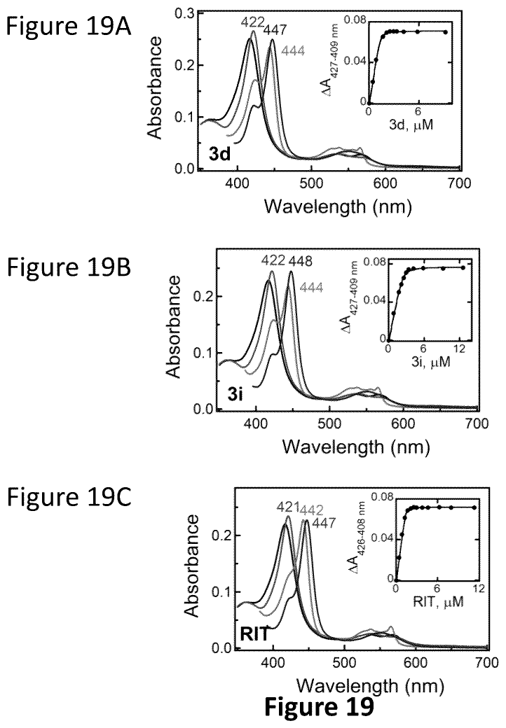

Crystallization of 3a-d-bound CYP3A4 All compounds willingly co-crystallized with CYP3A4 in the I222 crystal form, which had one molecule per asymmetric unit. Ligand fitting for compounds 3b and 3c was relatively straightforward. The compounds 3a and 3d binding modes, however, could not be accurately determined due to partially discontinuous electron density maps, likely the result of thermal disorder. The wild type (WT) CYP3A4-3a complex was re-crystallized in the C2 space group, where one of the two molecules in the asymmetric unit (molecule A) was well-defined. In the C2 crystal lattice, the F-F′ fragment mediates intermolecular contacts and undergoes positional and conformational rearrangement (FIG. 17A through FIG. 17C). Nonetheless, these changes had no significant effect on the ligand conformation (FIG. 18). With compounds 3d, neither WT nor previously used K282A/K285A mutant (Kaur P et al., 2016, J. Med. Chem., 59:4210-4220) produced crystals in space groups other than I222. Therefore, two other high entropy surface residues, K421 and K424 (Kaur P et al., 2016, J. Med. Chem., 59:4210-4220), located in the loop region on the distal face of CYP3A4 were used instead. The K421A/K424A mutant was well expressed, could bind compound 3d equally tight (Kₛ of 0.019 μM vs. 0.013 μM for WT; FIG. 19), and produced the desired C2 crystals. Importantly, K421 and K424 do not form specific interactions with residues of the same or nearby molecules, and their substitution with alanine does not alter the 400-430 fragment conformation (FIG. 17D).

Compounds 3a-d Binding Modes

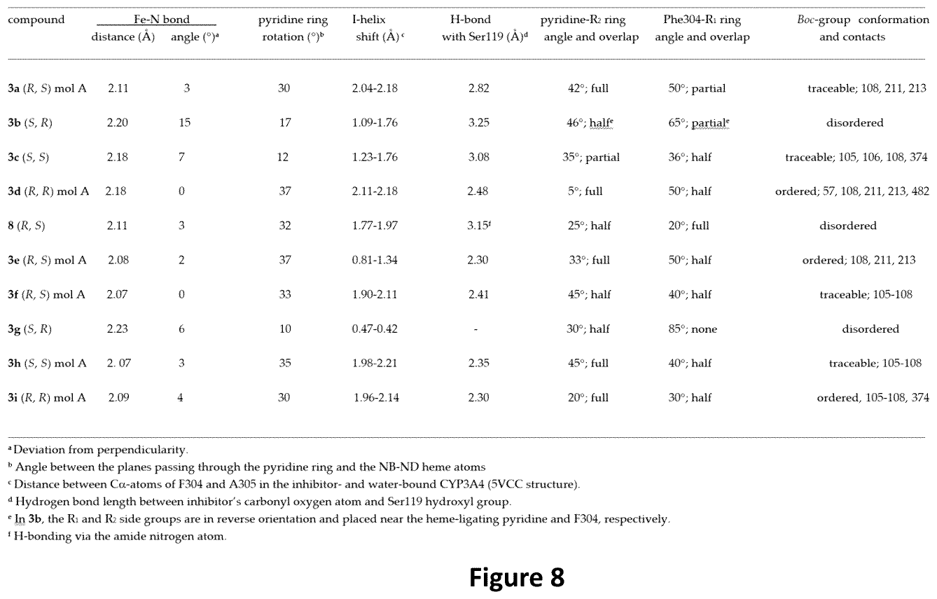

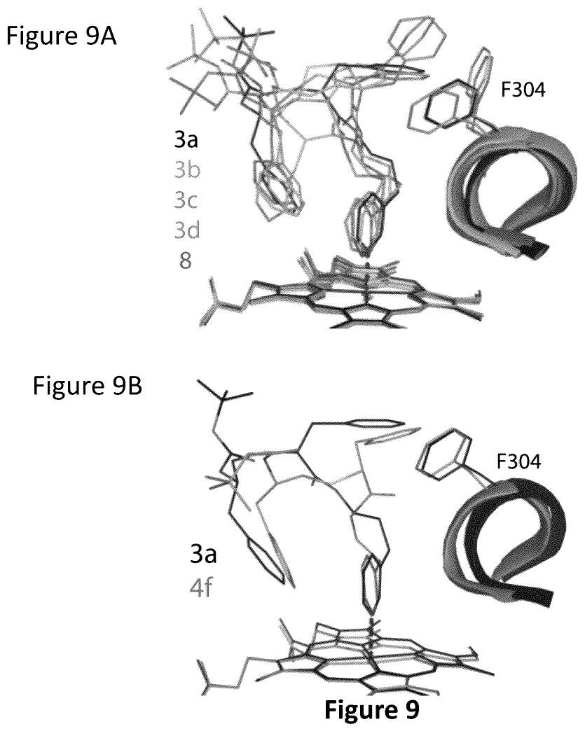

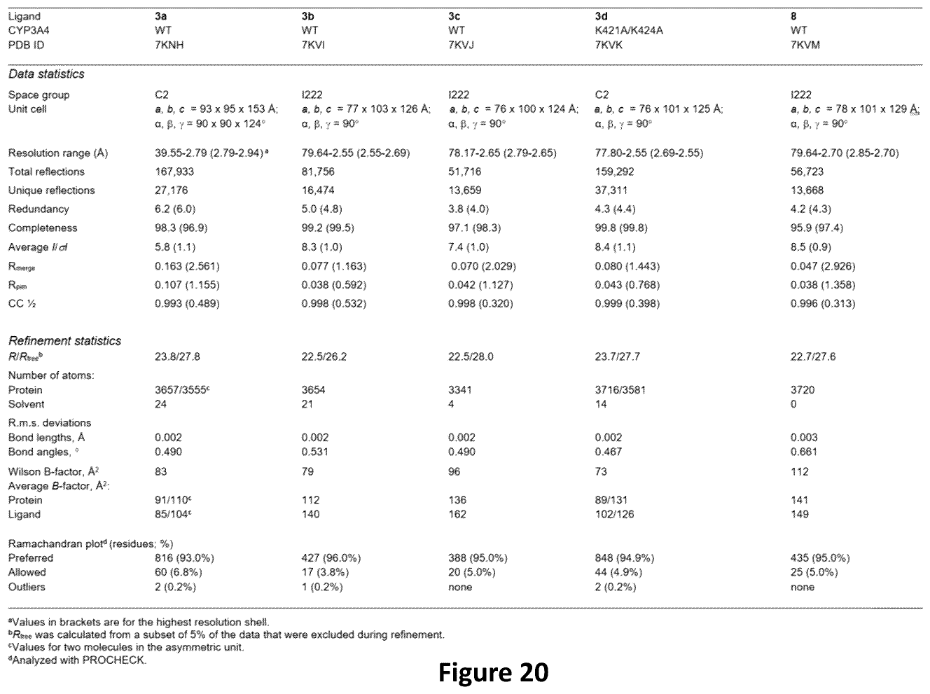

Structures of CYP3A4 bound to compound 3a-d were solved to 2.55-2.8 Å resolution (FIG. 20). Only the well-defined molecules A of 3a- and 3d-bound complexes were used for structural comparison, summarized in FIG. 8. Ligand positioning and orientation relative to the central I-helix, as well as omit electron density maps are shown in FIG. 6F through FIG. 6I. Compounds 3a, 3c, and 3d bound in a traditional orientation, with the R₁-phenyl embedded into a hydrophobic pocket above the I-helix (P1 site) and R₂-phenyl near the heme-ligating pyridine (P2 site). In contrast, compound 3d ligated to CYP3A4 in a reverse orientation, with R₁ and R₂ at the P2 and P1 sites, respectively (FIG. 6G), just like its methyl- and ethyl-linker counterparts (Samuels E R et al., 2019, Biochemistry, 58:2077-2087). The S/R conformation was less favorable for the binding to CYP3A4, as compound 3d formed the longest Fe—N bond and the weakest H-bond with the active site S119, a prerequisite for potent inhibition (Samuels E R et al., 2018, Mol. Pharm., 15:279-288). This translates to the lowest binding affinity, inhibitory potency, and stabilizing effect of compound 3d (FIG. 7). Even so, superposition of compounds 3a-d showed that, regardless of the side-group stereochemistry, phenyls at the P2 site virtually coincide (FIG. 9A). Being equally close to the heme and pyridine (˜3.8-4.2 Å), the side-groups were optimally poised for hydrophobic and aromatic interactions. The nearby R105 side chain (displayed in FIG. 6F through FIG. 6J) further stabilized the inhibitory complexes by promoting π-cation interactions with the aromatic R₂. Comparison of the subseries led compound 3a with the shorter compound 4f stereoisomer (Samuels E R et al., 2019, Biochemistry, 58:2077-2087) demonstrated how a one-atom linker extension altered the ligand binding mode (FIG. 9B). The longer pyridine-R₁ spacer allowed compound 3a to adopt a more relaxed conformation, with a larger tilt toward the heme. This strengthened the Fe—N bond (2.1 vs. 2.3 Å in compound 4f) and increased overlap with the heme macrocycle. Additional stabilization was provided by the terminal Boc-group, mediating multiple van der Waals interactions but disordered in compound 4f.

R₁/R₂ Spacer Extension had No Beneficial Effect

Compound 8 (rac, S) resembled compounds 3a-d but had a one-atom longer R₁-R₂ spacer. Spectral, biochemical, and kinetic properties of compound 8 were mostly similar (FIG. 7). The only distinction was a slightly lower ΔTₘ (5.0° C.) and incomplete heme reduction in the inhibitory complex: ˜60% vs.≥80% for compounds 3a-d (compare green spectra in FIG. 6A through FIG. 6E). In the crystal structure, compound 8 ligated to the heme in a traditional orientation (FIG. 6J). Importantly, stereochemistry of racemic R₁-phenyl was not specified during the first refining cycle but it was assigned as R by the structure refinement program. The R/S configuration was later confirmed to provide a better fit. That the five-atom R₁-R₂ spacer was less optimal for the binding to CYP3A4 was evident from the inability of compound 8 to strongly H-bond with S119 (FIG. 8). Moreover, the latter interaction involved the amide nitrogen rather than carbonyl oxygen, recruited in other analogues. Thus, the R₁/R₂ spacer extension had no beneficial effect.

Two-Atom Pyridyl-Linker Elongation was Detrimental for the Binding to CYP3A4

### Synthetic Approaches for Linker Elongation

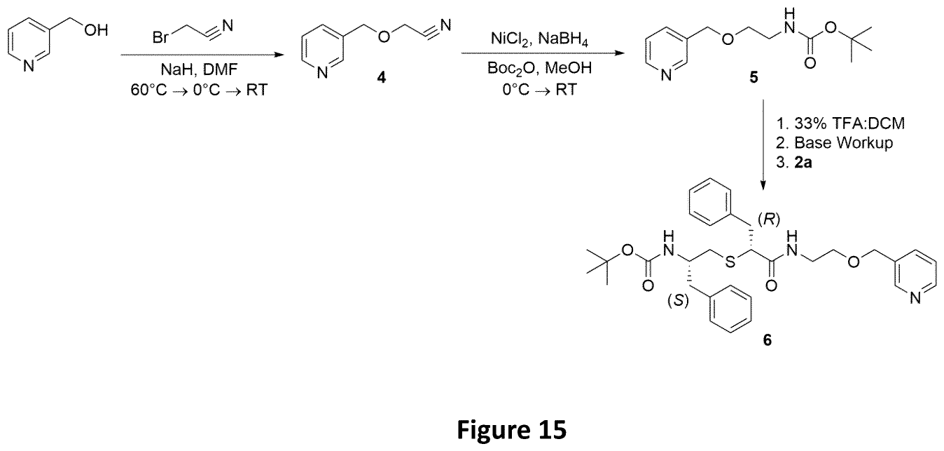

To further investigate the correlation between length/flexibility and binding affinity/inhibitory potency, a butyl-like linker was evaluated. For preliminary examination, a methoxyethyl linkage within the starting material was chosen as a bioisostere. Attempts to synthesize starting material with either pyridine-3-methanol and 2-(Boc-amino)ethyl bromide (Tang W et al., 2015, J. Am. Chem. Soc., 137:5980-5989) or 3-picolyl chloride (International Patent Application Publication No. WO 2008119741 A2) and Boc-ethanolamine, under various conditions, were either unsuccessful or produced product in very low yield. Moreover, the 3-picolyl chloride starting material was recovered from the synthesis, suggesting either low reactivity of chloride in this substitution, or diminished activity due to insolubility of the hydrochloride salt starting material. Because ether formation with the Boc-protected amino starting material was problematic, bromoacetonitrile was utilized instead. The nitrile product 4 (FIG. 15) was formed in more suitable yield. Several reduction methods were then attempted to reduce the nitrile to the amine. Reduction using LiAlH₄ under various conditions (Becker P et al., 2017, Angewandte Chemie, 56:8004-8008) was unsuccessful, as was electron transfer reduction via samarium (II) iodide (Szostak M et al., 2014, Organic letters, 16:1092-1095). H₂/Raney Nickel with NH₄OH (International Patent Application Publication No. WO 2013155388 A1) over-reduced the product to piperidine, and attempts to control reduction by shortening reaction time (Guan A et al., 2017, J. Agric. Food Chem., 65:1272-1280) were ineffective. Transition metal borides formed in situ were then employed. Zirconium boride, formed in situ from ZrCl₄ and NaBH₄ has been previously shown to reduce nitriles to amines (Itsuno S et al., 1988, Synth. Commun., 995-996), but was unsuccessful. However, nickel boride (formed in situ from NiCl₂ and NaBH₄) with Boc-anhydride as a trapping agent (Caddick S et al., 2000, Tetrahedron lett., 41:3513-3516), formed the Boc-protected amino product 5. Although catalytic quantities of nickel chloride have been shown to reduce nitriles to amines (Caddick S et al., 2003, Tetrahedron, 59:5417-5423), the reaction was more successful with stoichiometric amounts of nickel chloride hexahydrate and nitrile (Caddick S et al., 2000, Tetrahedron lett., 41:3513-3516; Khurana J M et al., 2002, Synth. Commun., 32:1265-1269). Overreduction to the piperidine product did occur, but was successfully separated by column chromatography. Deprotection of Boc with TFA resulted in free amine, which was then coupled with compound 2a to form the final methoxyethyl, butyl-like product 6.

Properties of Compound 6

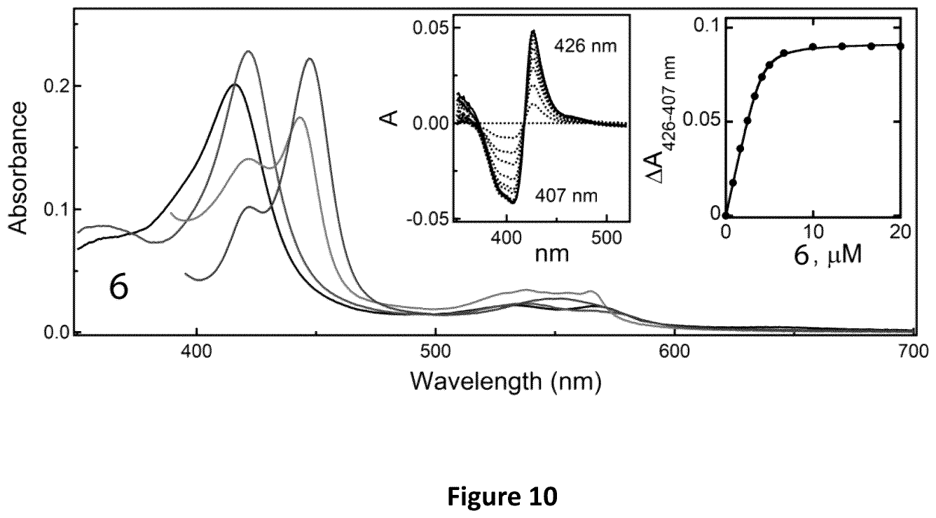

Similar to other analogues, compound 6 (R, S) acted as a type II heme ligand and induced a red shift in the Soret band (FIG. 10). Nonetheless, compound 6 was the weakest binder and the least potent inhibitor in the entire series, with Kₛ and IC₅₀ increased by multi-fold (FIG. 7). That compound 6 ligated weakly to CYP3A4 was also evident from the lowest ΔTₘ (<3° C.). Moreover, CYP3A4 could not be co-crystallize with compound 6 because it dissociated during crystallization. The inability of compound 6 to strongly ligate to the heme could be due to spatial limitations in the active site and/or conformational constraints imposed by the lengthy linker. Therefore, no further modification of the pyridine-R₁ spacer were pursued and rather focused on a more detailed investigation of the pyridyl-propyl scaffold by replacing R₂-phenyl with the larger and more hydrophobic indole and naphthalene.

Compounds 3e-i Subseries

Spectral and Biochemical Properties

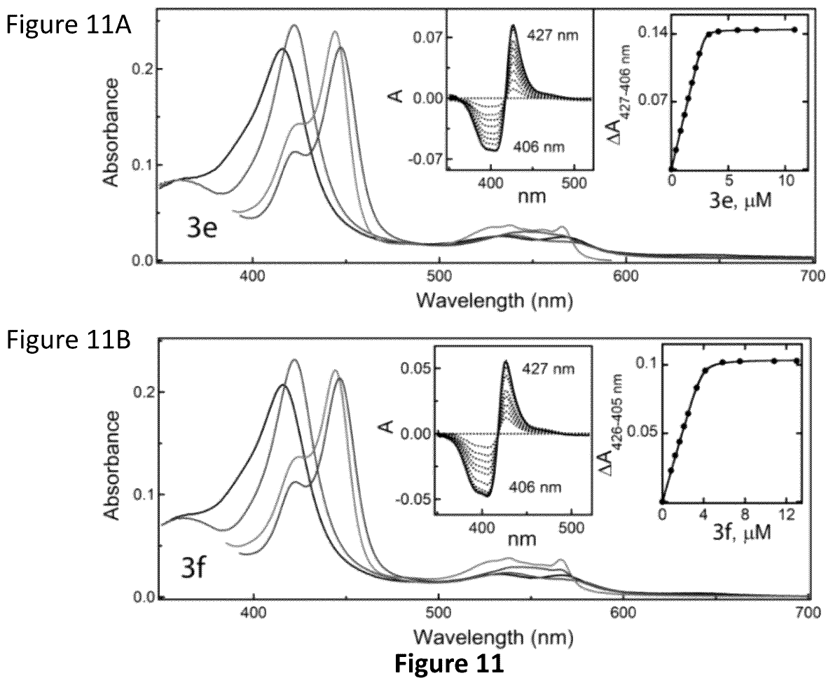

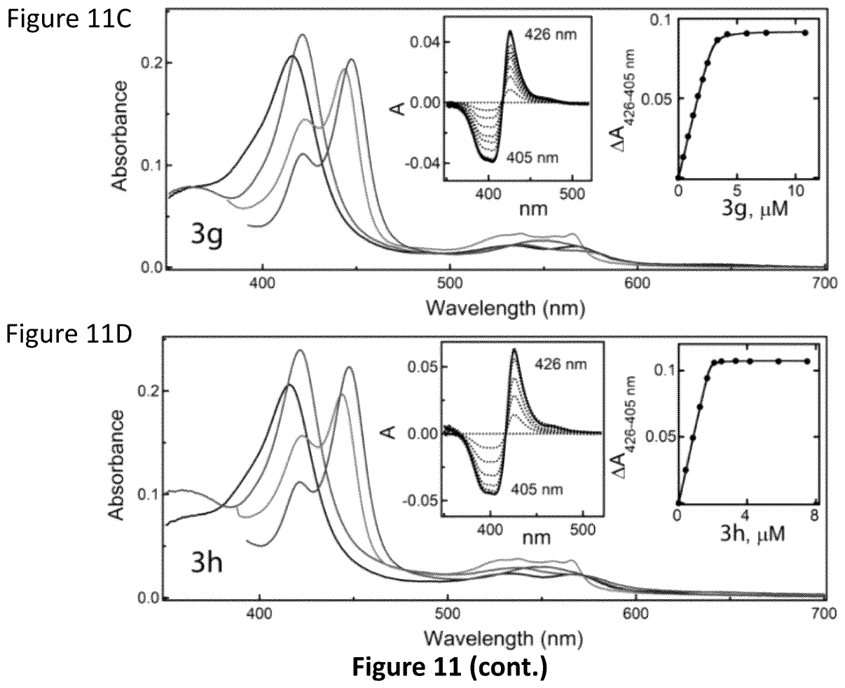

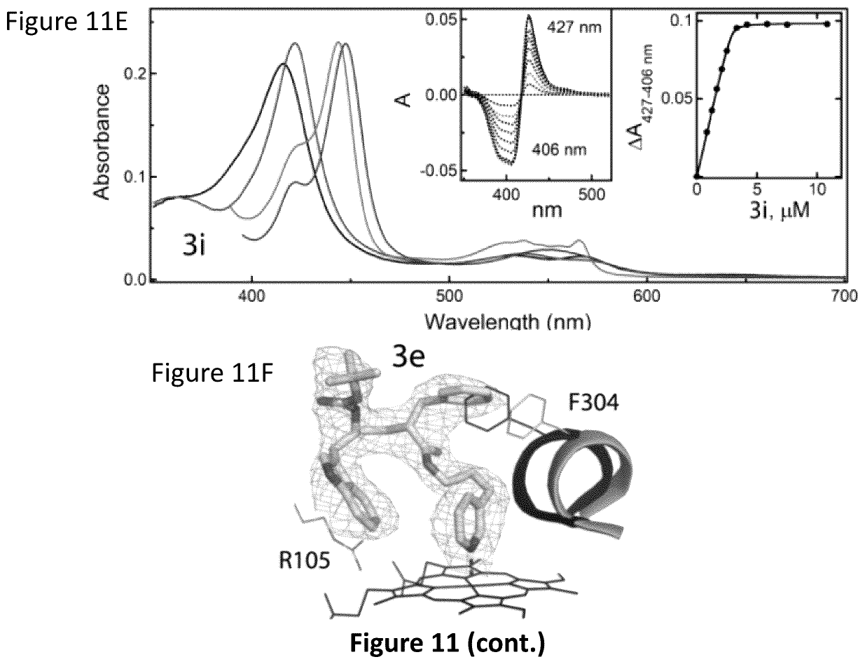

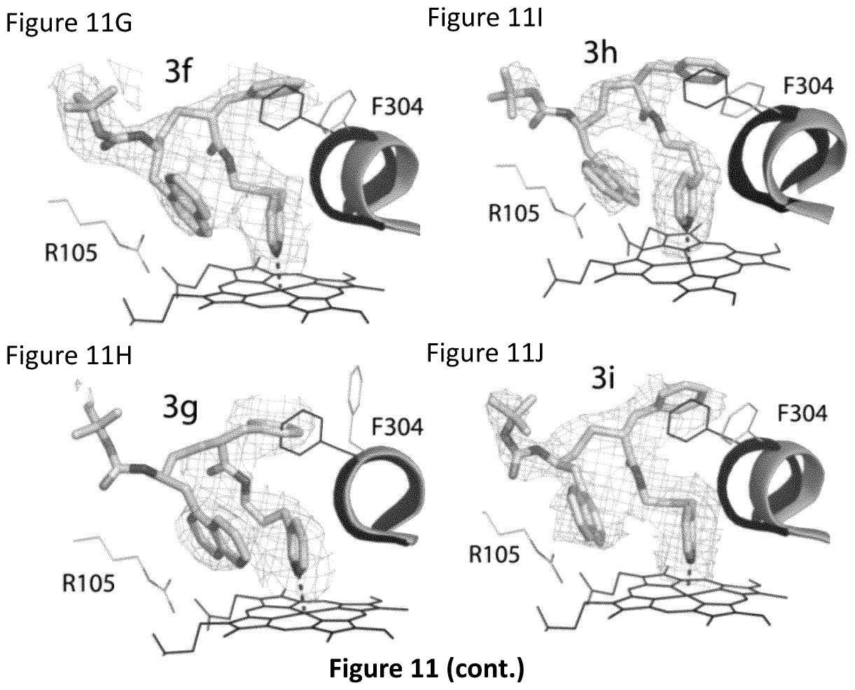

Absolute and difference absorbance spectra and titration plots for the compounds 3e-i subseries are shown in FIG. 11A through FIG. 11E. Compound 3e (R, S) was the only R₂-indole containing analogue investigated and, along with compound 3f (R, S) and compound 3i (R, R), was among the strongest binders and inhibitors of CYP3A4: Kₛ and IC₅₀ of 0.0015-0.019 μM and 0.15-0.016 μM, respectively. However, none of these compounds was superior to the R₂-phenyl containing compound 3a (FIG. 7). Comparison of compounds 3e and 3f with the shorter pyridyl-ethyl counterparts (compounds 7b and 7d from series IV; Samuels E R et al., 2020, Bioorg. Med. Chem., 28:115349-115360, respectively) showed that significant improvement in Kₛ was achieved only for compound 3e (4-fold decrease), while the IC₅₀ value and both parameters for compound 3f were either higher or unchanged. As expected, the S/R conformer, compound 3g, was the weakest inhibitor in the subseries and similar to compound 3b (FIG. 7). Compound 3h (S, S), in turn, was the series V lead compound: Kₛ and IC₅₀ of 0.007 μM and 0.090 μM, respectively. This was a 3-fold improvement relative to the R₁/R₂-phenyl containing stereoisomer compound 3c. Thus, the impact of phenyl-to-naphthalene replacement in R₂ depends on the side-group stereochemistry and is more pronounced when configuration is S/S.

Compounds 3e-i Binding Modes

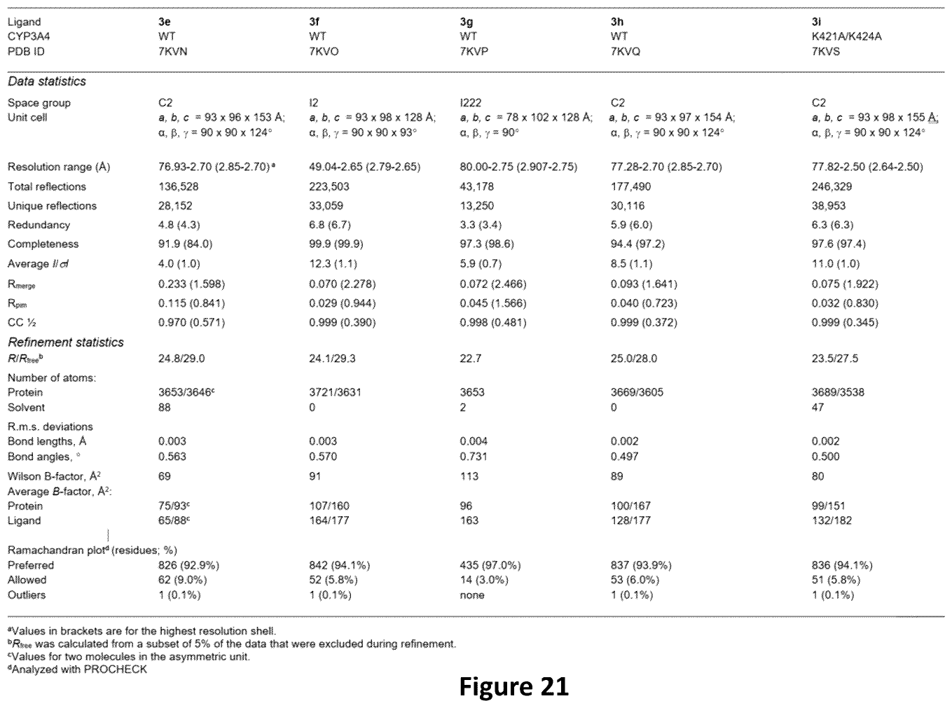

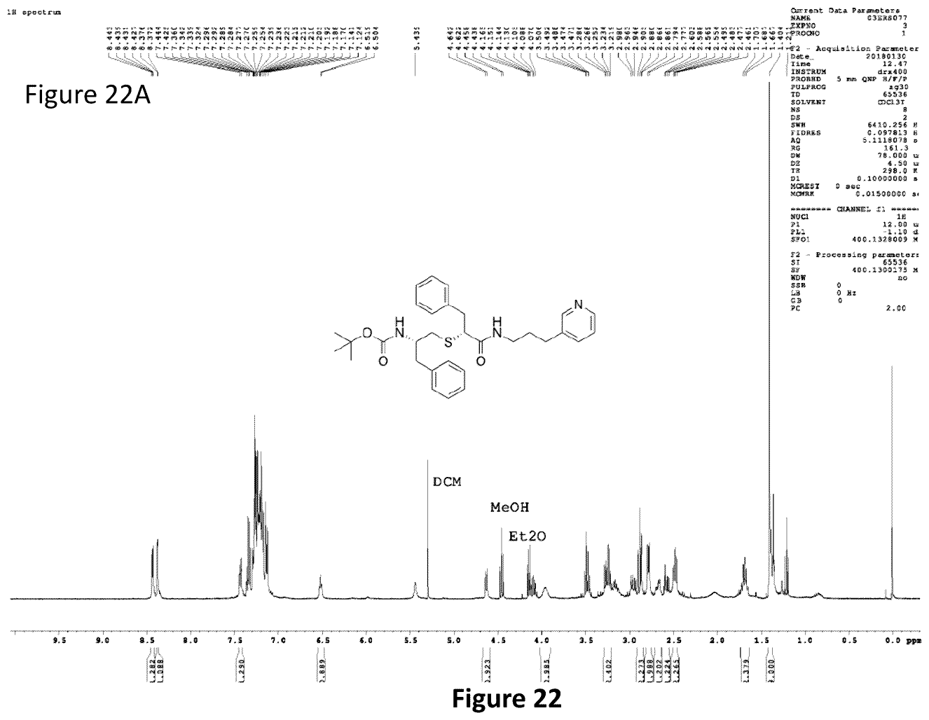

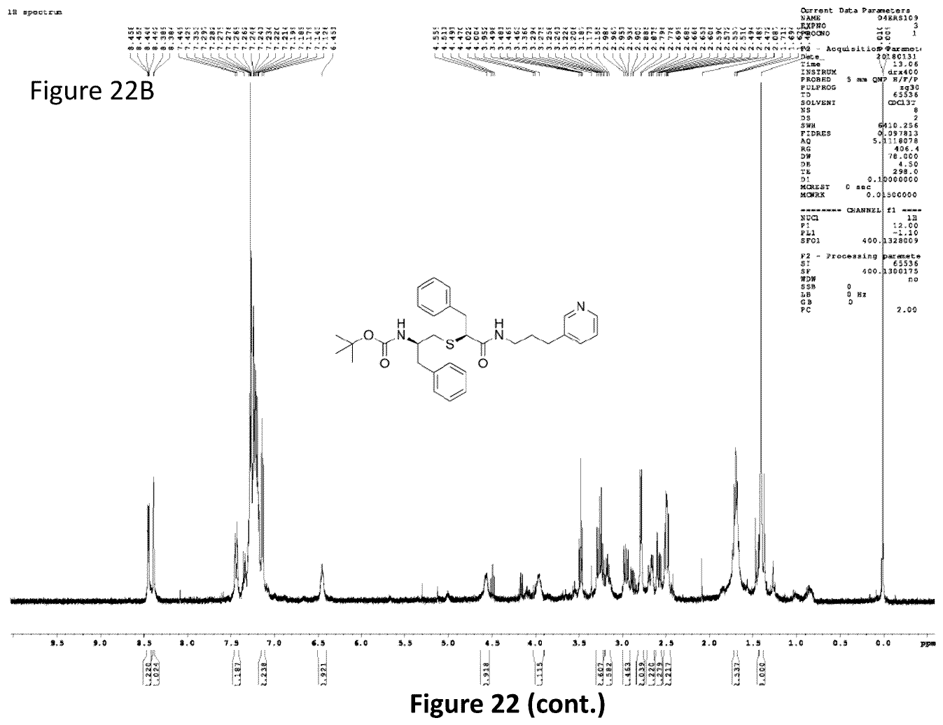

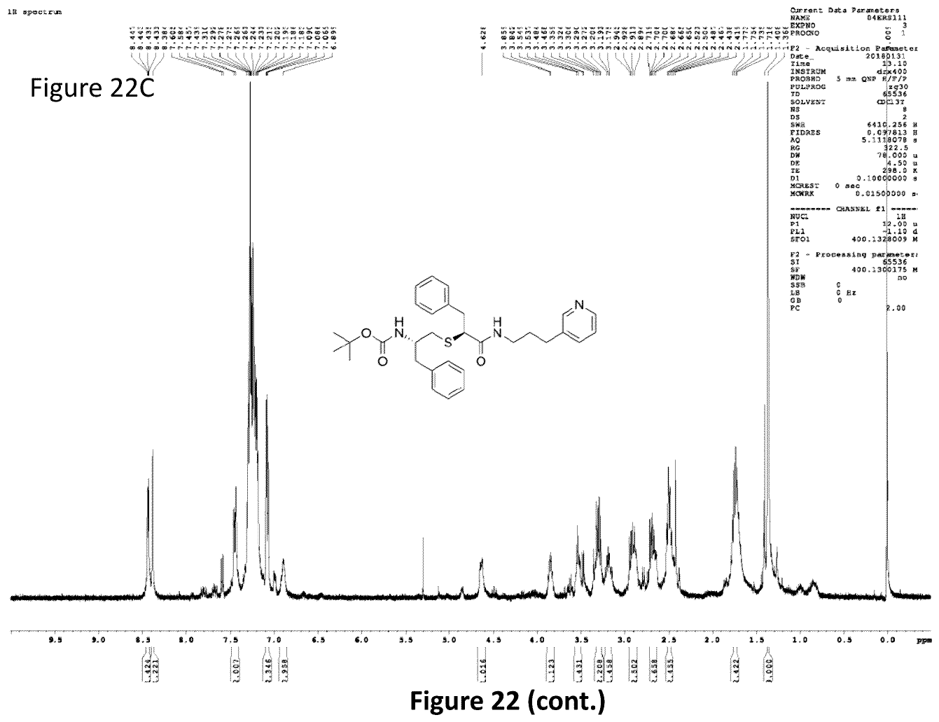

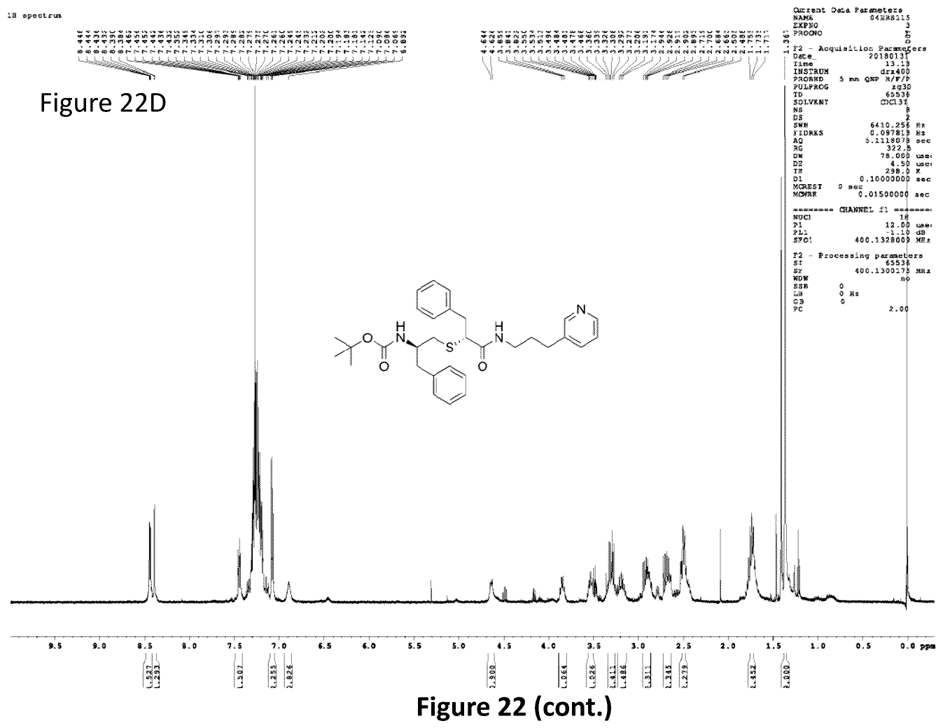

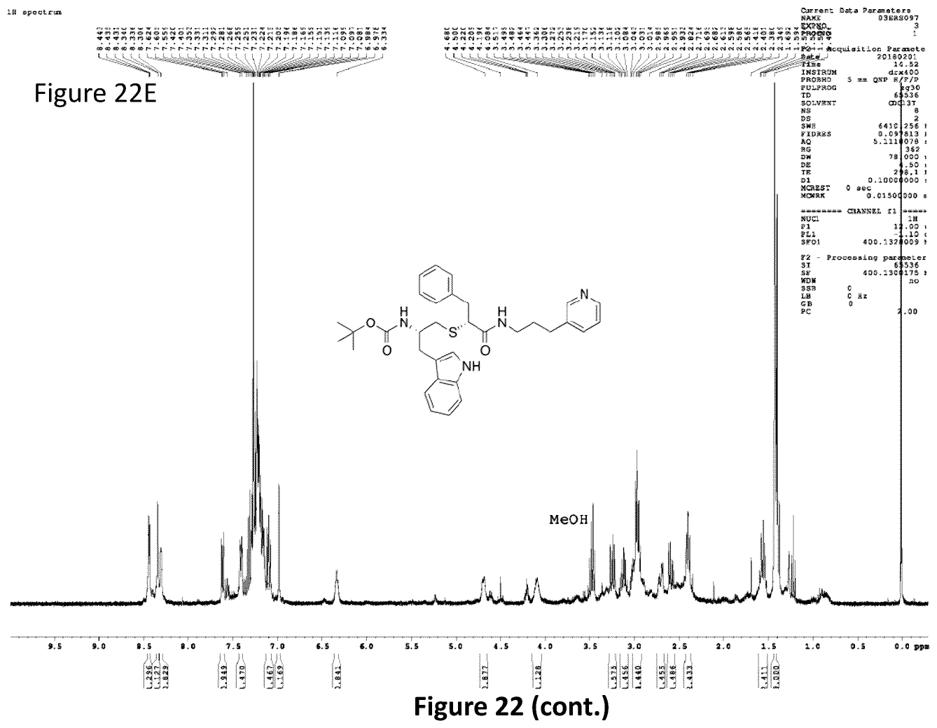

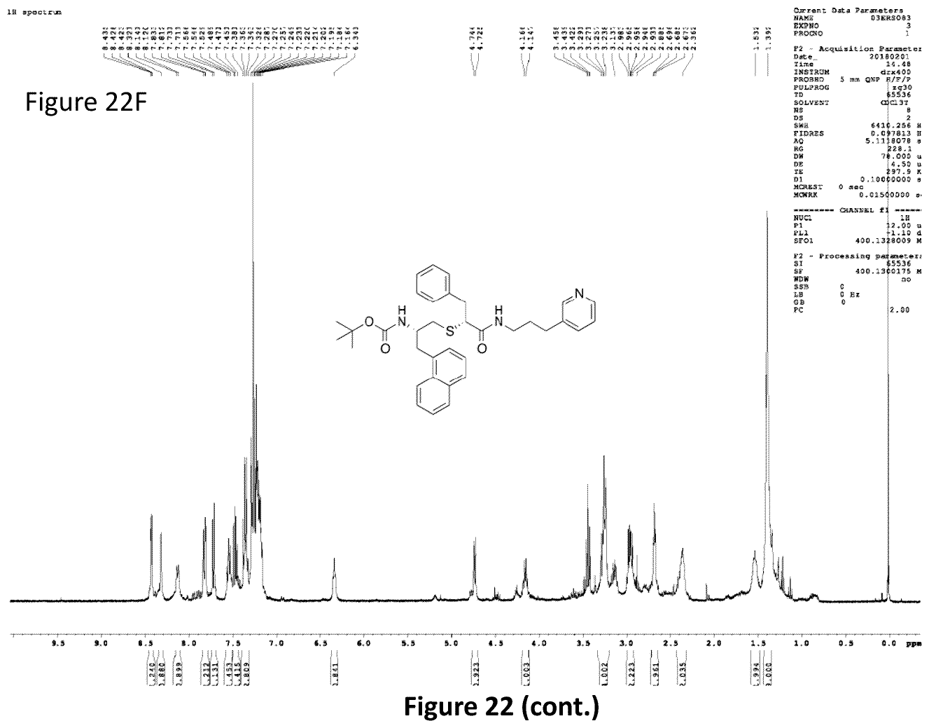

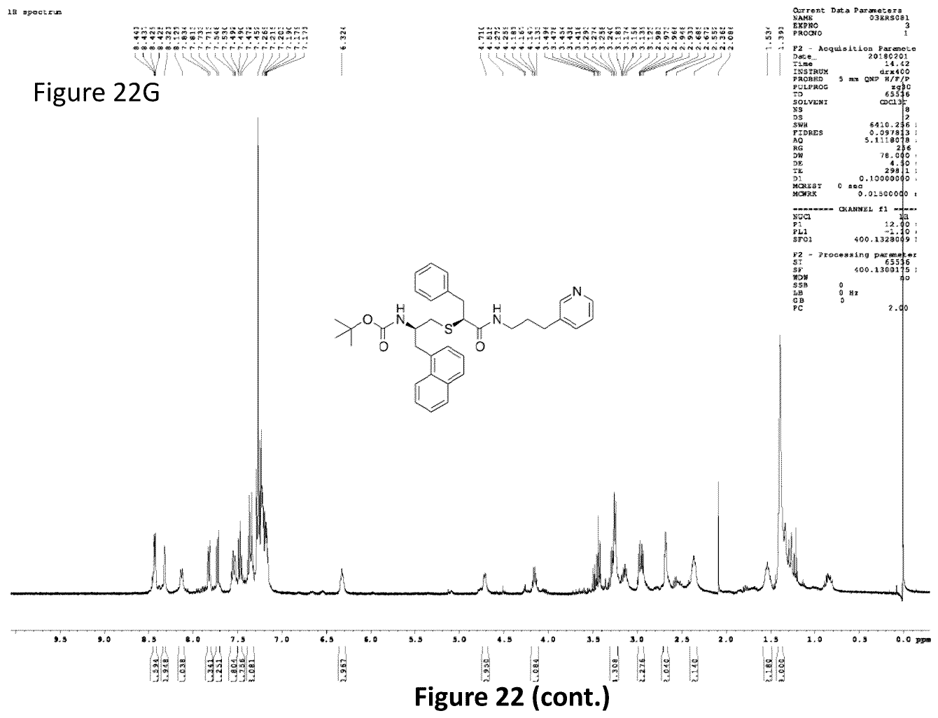

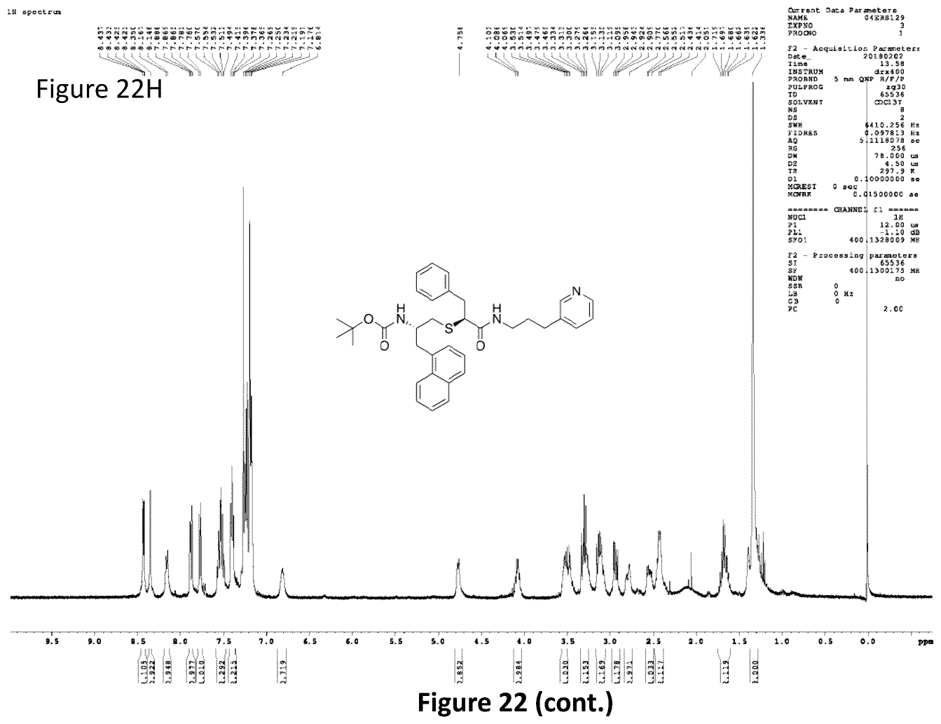

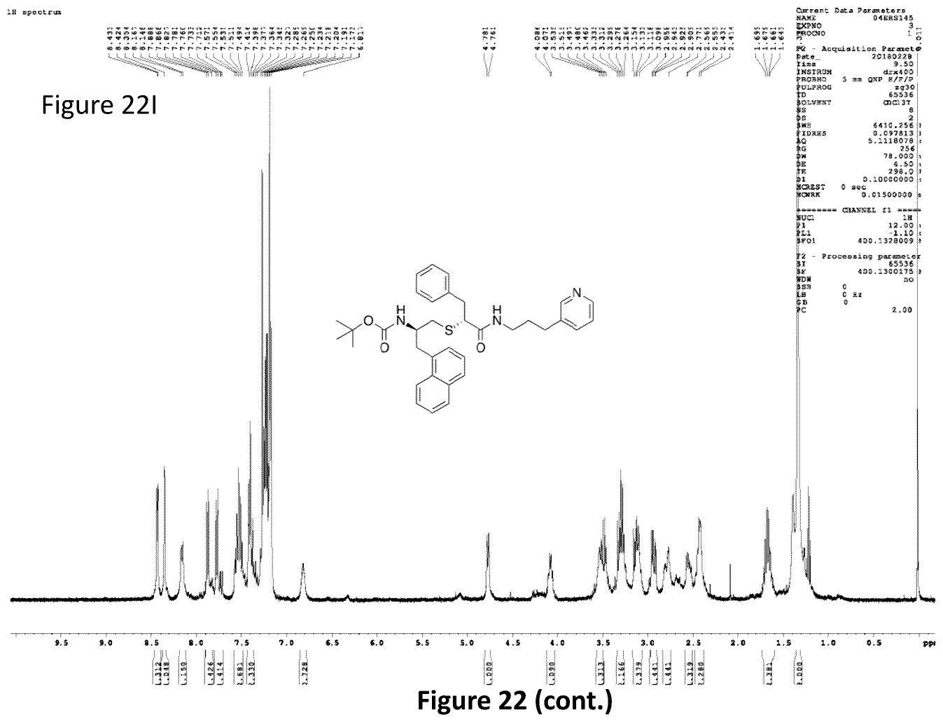

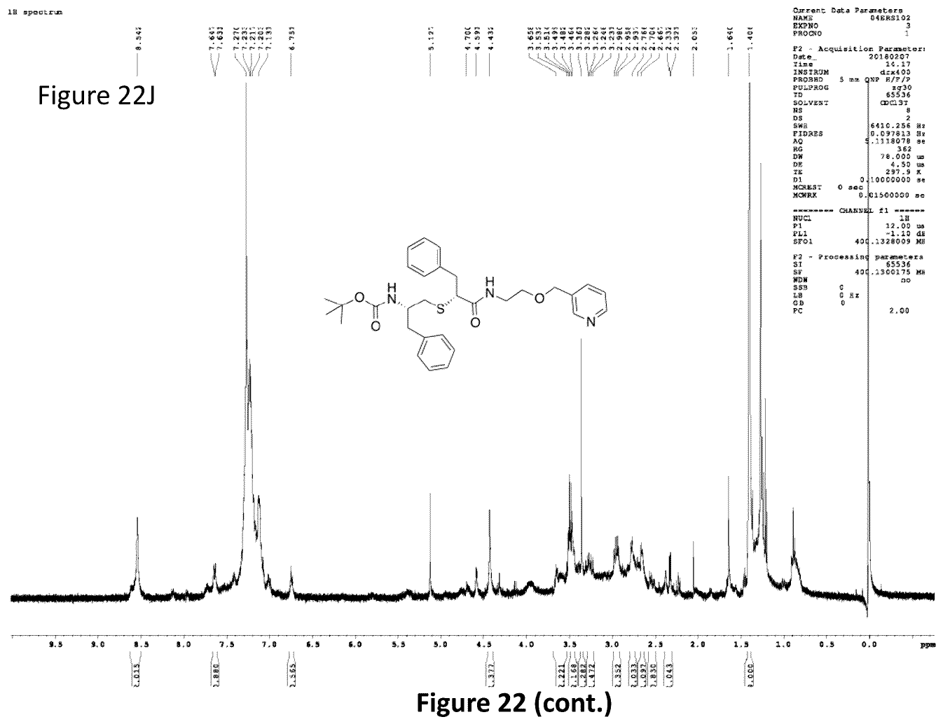

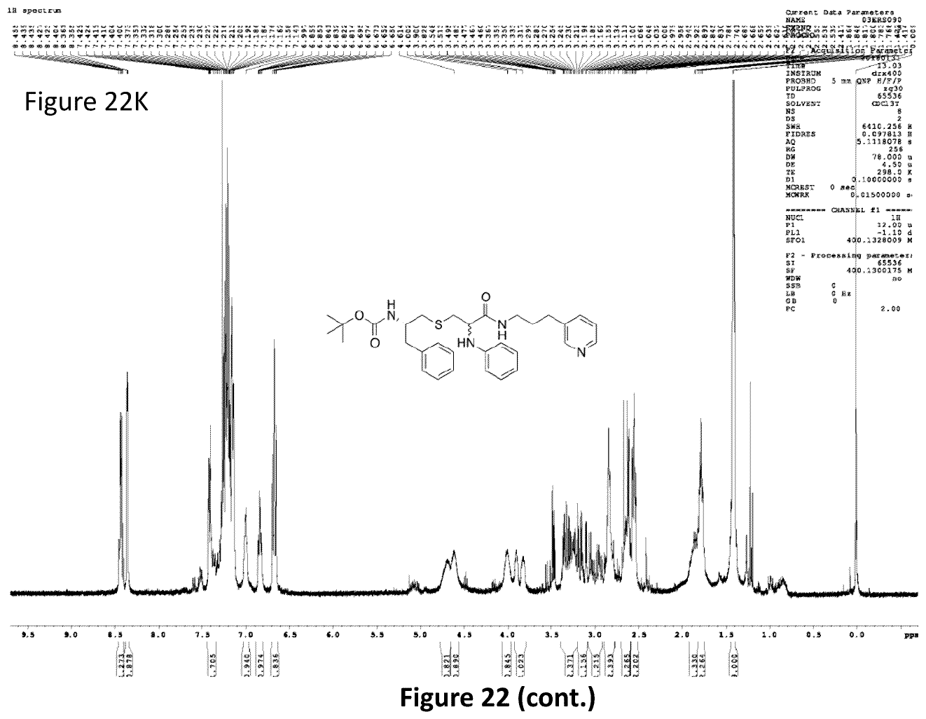

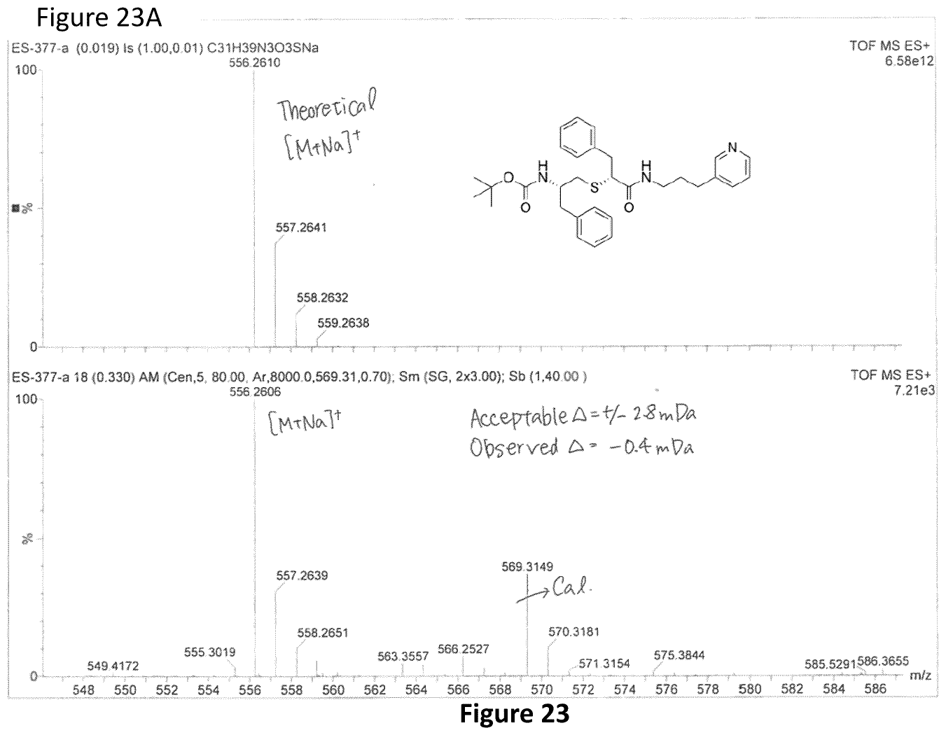

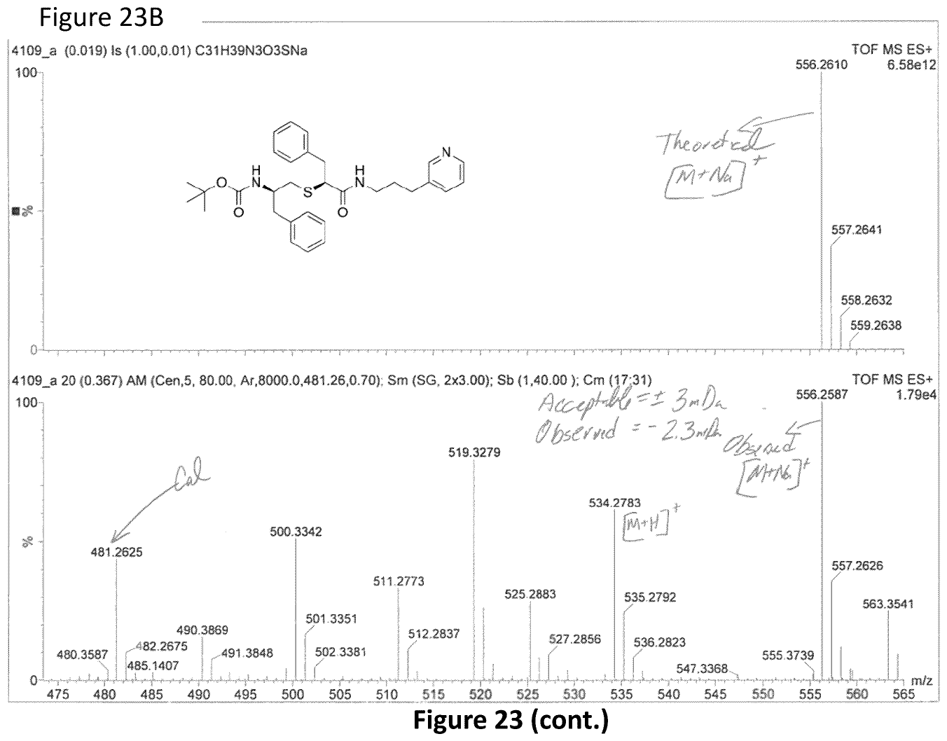

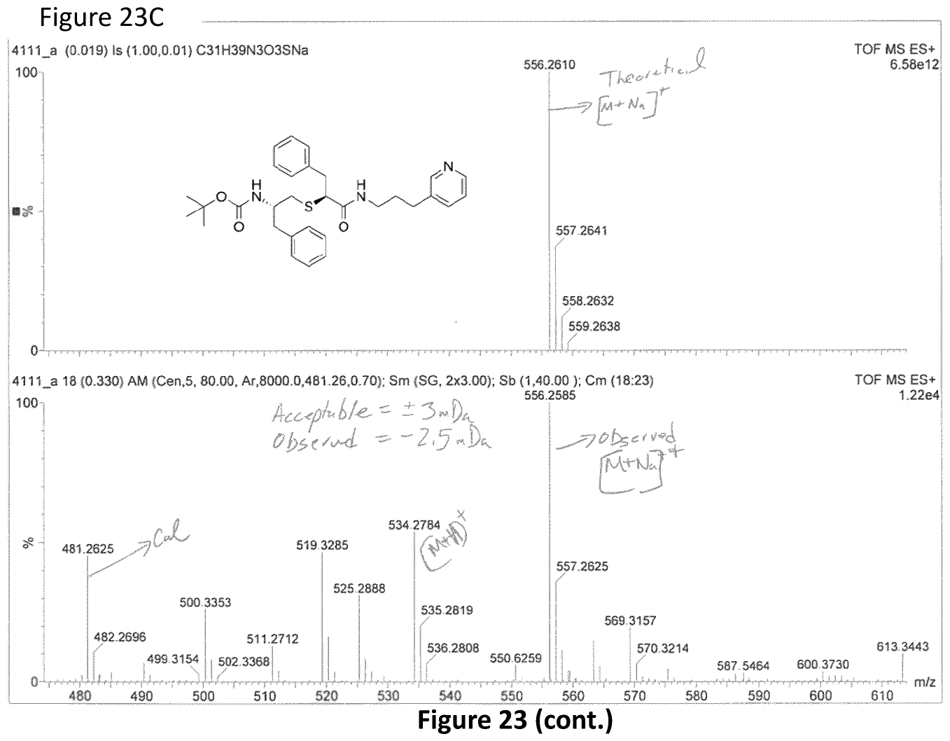

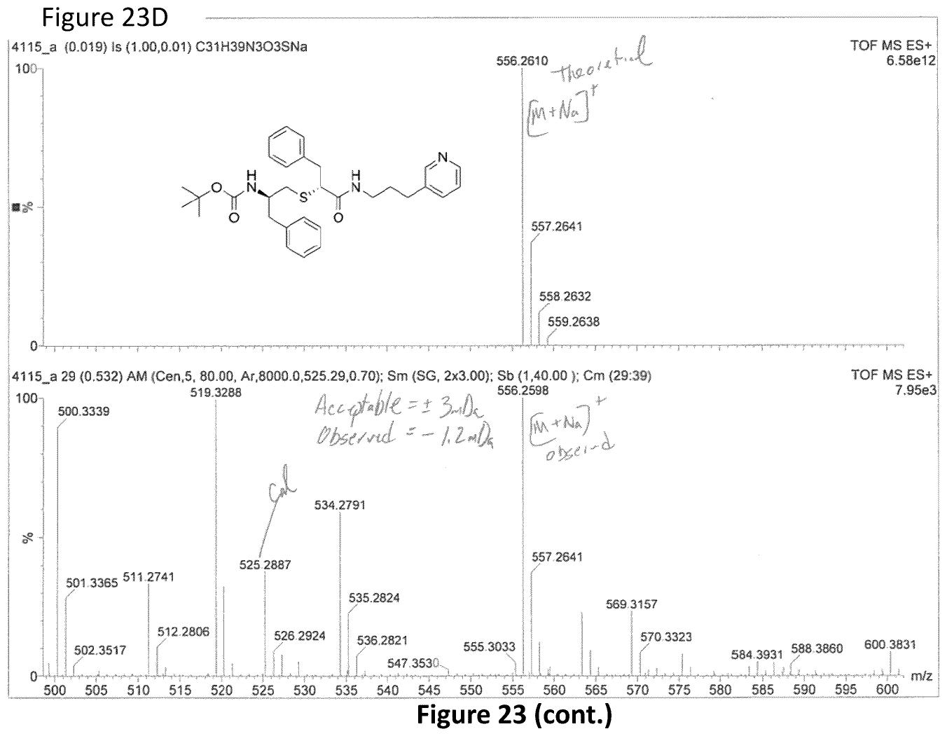

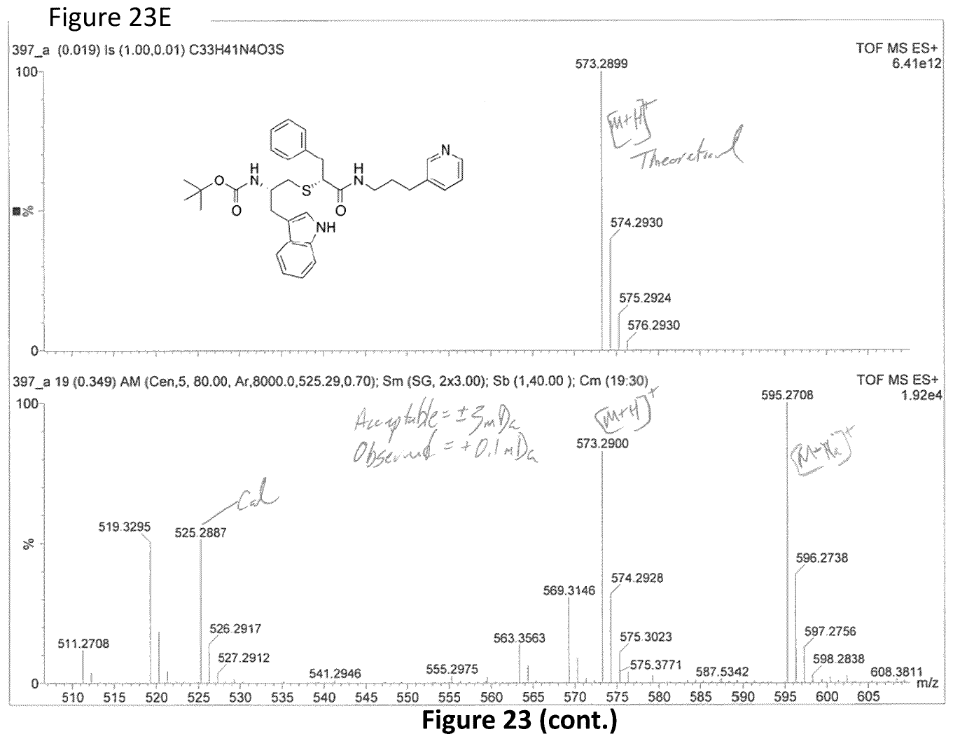

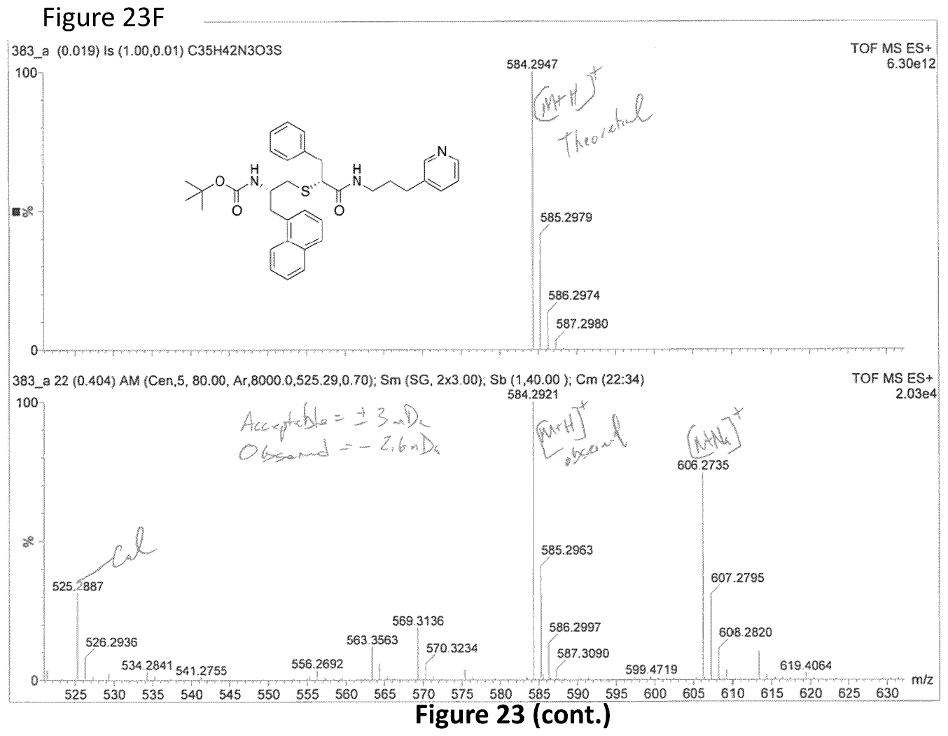

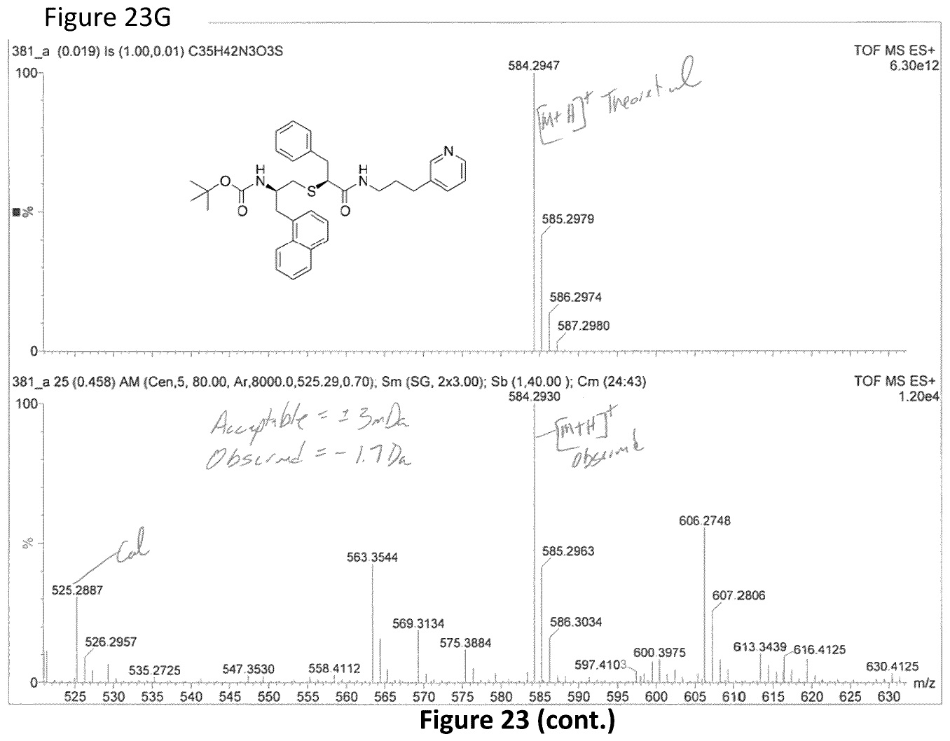

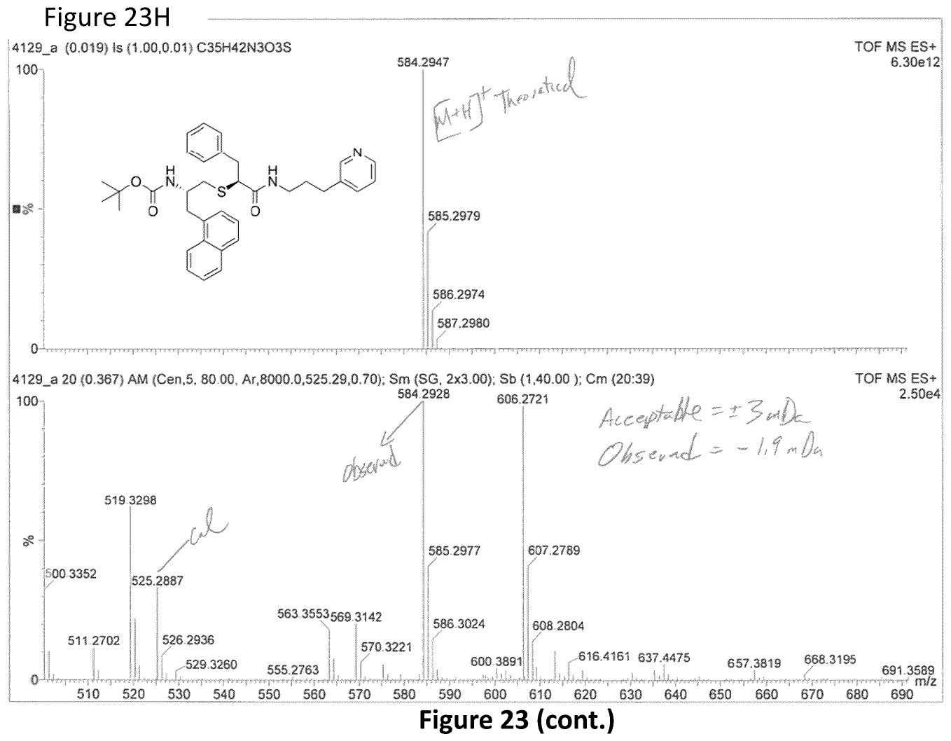

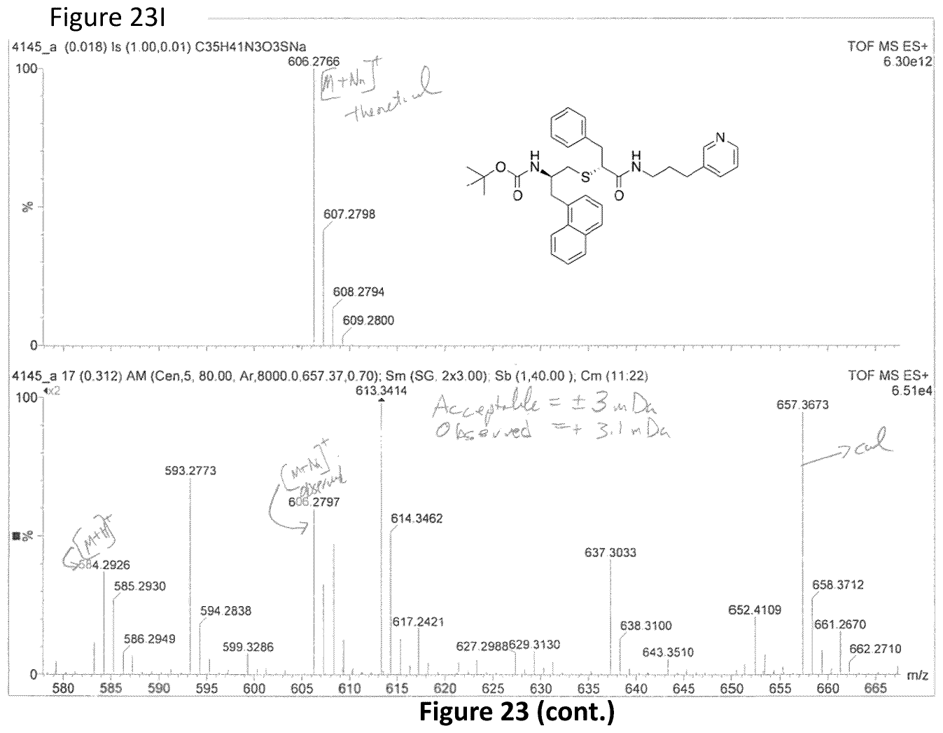

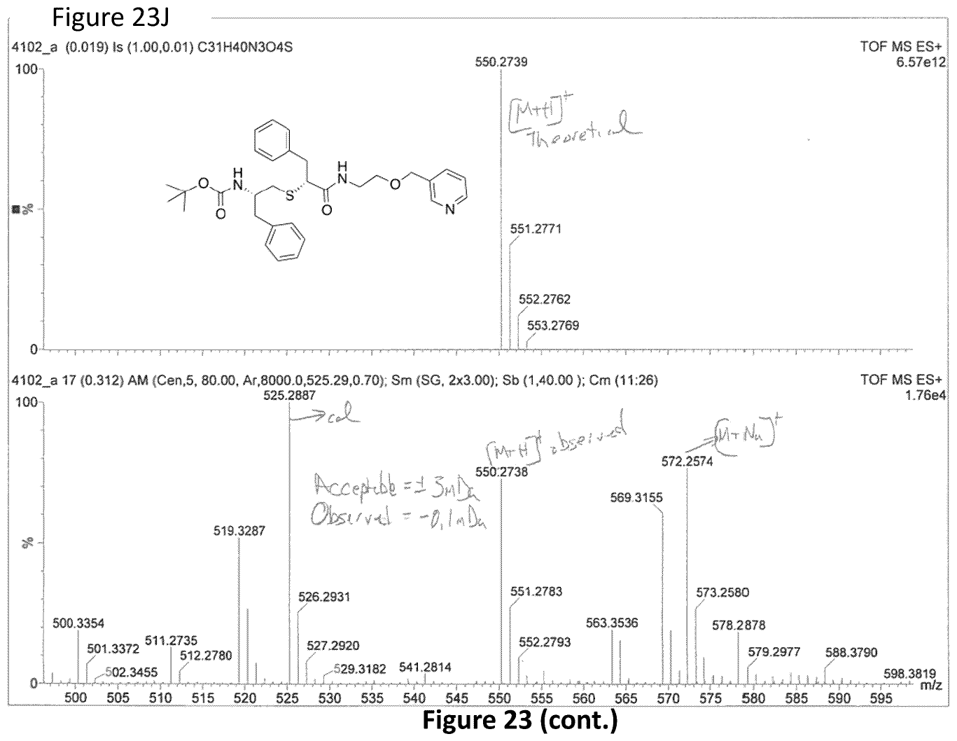

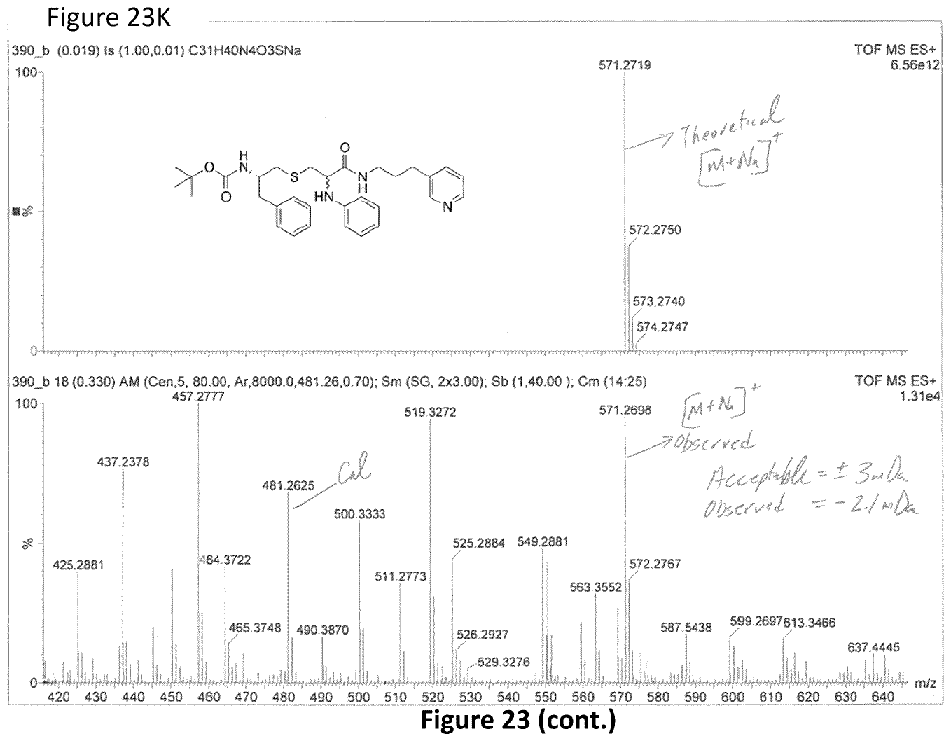

All compounds could be co-crystallized with WT CYP3A4 either in 1222, C2 or I2 space groups (FIG. 21). Similar to C₂, the I2 space group had two molecules in the asymmetric unit, where only molecule A was well defined. Compound 3i was poorly seen in the I222 structure and did not form C2 crystals with WT CYP3A4. Therefore, like compound 3d, this analogue was co-crystallized with the K421A/K424A mutant. The ligand binding modes and structural features were compared in FIG. 1 IF through FIG. 11J and FIG. 8. One distinction of this subseries was that all stereoisomers, including compound 3g, ligated to the heme in a traditional orientation, with R₁-phenyl in the P1 pocket and R₂-indole/naphthalene at P2 site. Still, even in this orientation, compound 3g had the longest Fe—N bond, cannot H-bond to S119, and had no R₁/F304 overlap. This was in contrast to four other compounds, which strongly ligated to the heme, formed strong H-bonds with S119, and maximized the R₁/F304 and R₂/pyridine overlap (FIG. 8).

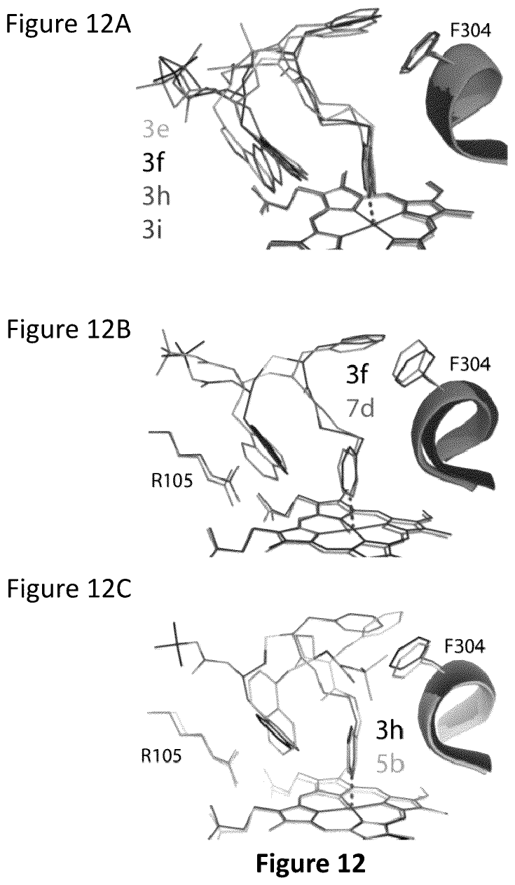

Superposition of compounds 3e, 3f, 3h, and 3i showed similar backbone curvatures, overlapping R₁-phenyls, and the R₂-groups equally distant from the heme but not the pyridine moiety (FIG. 12A). In compound 3e, the R₂-indole was oriented vertically rather than horizontally, as naphthalene rings did, possibly due to its more hydrophilic nature. Since compounds 3f and 3h bound in a similar manner, it was not possible to deduce based on structural and experimental data why the latter analogue inhibited CYP3A4 twice as stronger. Comparison of compound 3f with the shorter and isosteric compound 7d (FIG. 12B) also could not explain the 2-fold difference in IC₅₀, as the inhibitors' head-, side- and tail-groups were positioned similarly. On the other hand, compound 5b (S, S), selectively chosen by CYP3A4 from racemic mixture (Samuels E R et al., 2020, Bioorg. Med. Chem., 28:115349-115360), and the isosteric and equally potent compound 3h had notably distinct conformations (FIG. 12C): the 5b R₁ inserted deeper in the P1 pocket, R₂ oriented vertically relative to the heme, and the end-moiety pointed away rather than toward the substrate channel. Because it became challenging to rank the high affinity analogues based on traditional approaches, an additional assay for inhibitor characterization were introduced.

H₂O₂ Heme Accessibility Assessment

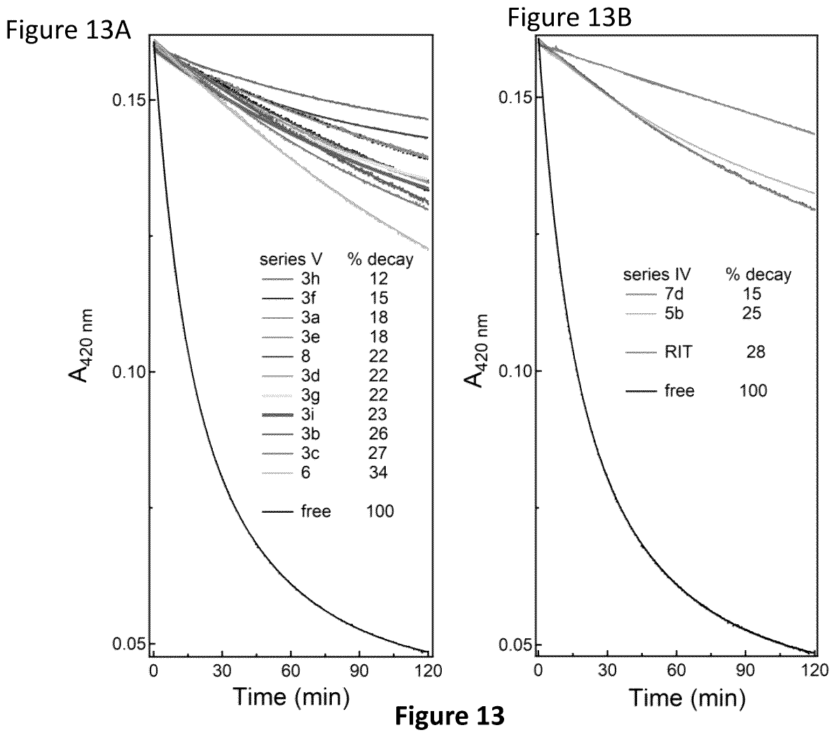

Ritonavir-like molecules inhibit CYP3A4 not only via heme ligation and decrease in the heme redox potential (Sevrioukova I F et al., 2010, Proc. Natl. Acad. Sci. USA, 107:18422-18427) but also by blocking the active site and preventing substrates from accessing the catalytic center. The heme accessibility were usually evaluated by measuring the rate and percentage of anaerobic heme reduction by sodium dithionite, an artificial electron donor (k^{ET} in FIG. 7). However, k^{ET} had a narrow spread, and the portion of heme reduced did not correlate with IC₅₀ (FIG. 7). Therefore, another method, heme bleaching with hydrogen peroxide, was utilized to probe accessibility of the cofactor. H₂O₂ is a small oxidizing agent that easily penetrates the protein interior and destroys the heme without altering the P450 structure (Pikuleva I A et al., 1992, J. Biol. Chem., 267:1438-1442). Kinetic plots for series V inhibitors are shown in FIG. 13A. Due to complex, multiphasic nature of heme decay kinetics, it was more convenient to compare the percentage of heme destroyed at the end of the reaction. In general, the strongest binders/inhibitors (compounds 3h, 3f, 3a, and 3e) preserved most of the heme (>80%), whereas the weaker ligands (compounds 3g, 3b, 3c, and 6) had a smaller protecting effect (4-16% higher heme loss). To further test the applicability of the assay, H₂O₂-dependent heme destruction in CYP3A4 bound to ritonavir and series IV analogues 5b and 7d was measured (FIG. 4). As seen from FIG. 13B, only the R₂-naphthalene containing compound 7d protected the heme as well as the best series V inhibitors. Interestingly, the fraction of heme destroyed seemed to better correlate with the Kₛ/IC₅₀ pair rather than the individual parameters. Either way, the heme depletion assay was informative and indicated that, along with the strong heme-ligating moiety, the R₂-mediated interactions at P2 site could control/minimize heme accessibility.

Overall, the present example depicts a continuation of ongoing efforts to identify structural attributes required for potent inhibition of human drug-metabolizing CYP3A4, the major and most clinically relevant P450 isoform. This knowledge was needed for better understanding of the CYP3A4 inhibitory and ligand binding mechanism, and could help with the development of safer drugs and more potent pharmacoenhancers. Ritonavir, originally designed as an anti-HIV drug (Kempf D J et al., 1995, Proc. Natl. Acad. Sci. USA, 92:2484-2488), remained the most potent CYP3A4 inhibitor in clinical use (Greenblatt D J et al., 2015, Br. J. Clin. Pharmacol., 80:342-350) and served as a ‘gold standard’ in the above-described studies. Based on findings on the closest analogues of ritonavir, a pharmacophore model for a potent CYP3A4-specific inhibitor were developed (Sevrioukova I F et al., 2014, Curr. Top. Med. Chem., 14:1348-1355) and tested some of the determinants, including the backbone length/composition, R₁ and R₂ substituents, and pyridine-R₁ and R₁-R₂ spacers (Kaur P et al., 2016, J. Med. Chem., 59:4210-4220; Samuels E R et al., 2018, Mol. Pharm., 15:279-288; Samuels E R et al., 2019, Biochemistry, 58:2077-2087; Samuels E R et al., 2020, Bioorg. Med. Chem., 28:115349-115360). This study built on the prior work and was set to elucidate the optimal length of the pyridine-R₁ linker.

The linker extension from pyridyl-ethyl to pyridyl-propyl (six- to seven-atom pyridine-R₁ separation) was found to be more optimal for the R₁/R₂-phenyl containing compounds 3a-d as it increased both the binding and inhibitory strength by several-fold (FIG. 7). For the R₁-phenyl/R₂-indole/naphthalene analogues, compounds 3e-i, the impact was not uniform and depended on the side-group stereochemistry. In accord with the previous findings (Samuels E R et al., 2019, Biochemistry, 58:2077-2087; Samuels E R et al., 2020, Bioorg. Med. Chem., 28:115349-115360), the S/R configuration was confirmed to be the least favorable, as it forced analogues to bind in reverse or sub-optimal orientation. In contrast, regardless of the scaffold, R/S was the most preferable conformation and, once again, was selected by CYP3A4 during co-crystallization with compound 8 (rac, S). The series V front-runner, however, was compound 3h (S, S). This analogue had outstandingly low Kₛ (0.007 μM), inhibited CYP3A4 nearly as potently as the prior lead compounds (IC₅₀ of 0.090 μM vs. 0.055-0.070 μM for compounds 5b and 7d) and 1.5-fold stronger than ritonavir (Kₛ and IC₅₀ of 0.019 μM and 0.130 μM, respectively).

The currently prevalent notion is that ritonavir acts as a mechanism-based inhibitor by producing a reactive metabolite(s) inactivating CYP3A4 via covalent attachment (Koudriakova T et al., 1998, Drug Metab. Dispos., 26:552-561; von Moltke L L et al., 2000, Eur. J. Clin. Pharmacol., 56:259-261; Ernest C S et al., 2005, J. Pharmacol. Exp. Ther., 312:583-591; Lin H L et al., 2013, Drug Metab. Dispos., 41:1813-1824; Rock B M et al., 2014, Mol. Pharmacol., 86:665-674). In the experimental system, the time-dependent CYP3A4 inactivation upon preincubation with NADPH could not detected and, instead, observed a slight decrease in IC₅₀ for ritonavir, likely due to partial metabolism (Samuels E R et al., 2019, Biochemistry, 58:2077-2087). This and the prior studies demonstrated that the key factors contributing to inhibition of CYP3A4 were the tight heme binding and blockage of the active site.

One problem with series V compounds was that, aside from the best and the worst inhibitors, the ranking was difficult due to low spread in IC₅₀ (FIG. 7). By definition, IC₅₀ is a half inhibitory concentration, which under the experimental conditions (0.1 μM CYP3A4; 1:1 enzyme-inhibitor complex) was limited to 0.05 μM. This explained the IC₅₀ clustering and predicted a similar scenario for the next generation of optimized compounds. Contrarily, Kₛ does not depend on protein concentration and, as demonstrated for compound 3h, its significant improvement can still be achieved. The newly introduced assay, H₂O₂-dependent heme bleaching, helped to better evaluate the heme accessibility in the inhibitor-bound CYP3A4 and demonstrated that, compared to the best series IV and V analogues, the protective effect of ritonavir was considerably lower. This further underlines the importance of strong heme ligation and fuller occupancy of the P2 site.

Another challenge was to accurately define the ligand binding modes. Despite the fact that most of new compounds were the tight binders that willingly co-crystallized with CYP3A4, it was difficult to determine their binding orientation in traditional I222 crystals, containing one loosely packed CYP3A4 molecule in the unit cell. This and the higher motional freedom of elongated compounds led to thermal disorder and discontinuous electron density maps. To overcome this problem, some but not all inhibitory complexes were recrystallized in more densely packed C2 and I2 space groups. As expected, changes in crystal packing triggered restructuring of surface loops, including the F-F′ fragment (FIG. 17A through FIG. 17C), which nonetheless only minimally distorted the ligand binding modes (FIG. 18). Two compounds, 3d and 3i, failed to co-crystallize with WT and the previously obtained K282A/K285A CYP3A4 (Kaur P et al., 2016, J. Med. Chem., 59:4210-4220) in the desired crystal forms. This prompted to clone another surface mutant, K421A/K424A, which had similar spectral and ligand-binding properties (FIG. 19), easily formed C2 crystals, and maintained the same structural fold (FIG. 17D). Thus, along with WT and K282A/K285A CYP3A4, this variant can be used in crystallization trials to increase chances for success.

Determination of all but 6-bound structures of CYP3A4 was highly important because it enabled structural comparison within/between subseries and with the pyridyl-ethyl counterparts (FIG. 9B, FIG. 12B, and FIG. 12C). Comparative analysis indicated that the positive impact of one-atom linker extension on Kₛ and IC₅₀ could be due to higher flexibility and adjustability of the elongated analogues, leading to stronger heme ligation and more relaxed fit. This, in turn, strengthened protein-ligand interactions, as manifested by a large increase in thermostability of CYP3A4 (ΔTₘ in FIG. 7). There was also a downside effect, an increase in ligand mobility and thermal motion (evident from the elevated B-factors; FIG. 20 and FIG. 21), that could lower the inhibitory potency to some extent. This possibility explained why most of series V analogues were inferior to the shorter compounds 5b and 7d. Thus, a balance needed to be found between the flexibility/adjustability and rigidity of the scaffold. One way to achieve this is through modification of the end-moiety, currently presented by a simple amino-protecting Boc-group.

In conclusion, spectral, functional, and structural characterization of eleven series V analogues showed that a one-atom head-group linker elongation, from six- to seven-atom pyridine-R₁ separation, was beneficial and improved Kₛ, IC₅₀ and thermostability of CYP3A4. In contrast, further linker extension lowered the binding and inhibitory strength by several-fold, likely due to spatial limitations in the active site and/or conformational constraints. Compared to the shorter series IV analogues, an increase in the R₂ size/hydrophobicity had a less pronounced impact, partly due to higher flexibility/mobility of the elongated compounds. Even so, the new lead compound 3h was among the best CYP3A4 inhibitors designed so far and with the highest binding affinity ever detected. Based on SAR analysis of series IV and V analogues, it was concluded that further improvement in the inhibitory power are likely to be achieved by balancing flexibility/adjustability and rigidity of the scaffold.

In summary, inhibition of the major human drug-metabolizing cytochrome CYP3A4 by pharmaceuticals and other xenobiotics likely lead to toxicity, drug-drug interactions and other adverse effects, as well as pharmacoenhancement. Despite serious clinical implications, the structural basis and attributes required for potent inhibition of CYP3A4 remain to be established. The present study utilized a rational inhibitor design to investigate structure-activity relationships in the analogues of ritonavir, the most potent CYP3A4 inhibitor in clinical use. This study elucidated the optimal length of the head-group spacer using eleven (series V) analogues with the R₁/R₂ side-groups as phenyls or R₁-phenyl/R₂-indole/naphthalene in various stereo configurations. Spectral, functional and structural characterization of the inhibitory complexes showed that a one-atom head-group linker elongation, from pyridyl-ethyl to pyridyl-propyl, was beneficial and markedly improved Kₛ, IC₅₀ and thermostability of CYP3A4. In contrast, a two-atom linker extension led to a multi-fold decrease in the binding and inhibitory strength, possibly due to spatial and/or conformational constraints. The lead compound, 3h (FIG. 5), was among the best inhibitors designed so far and, overall, the strongest binder (Kₛ and IC₅₀ of 0.007 and 0.090 μM, respectively). That 3h was the fourth structurally simpler inhibitor superior to ritonavir further demonstrated the power of the above-described approach.

The materials and methods employed in these experiments are now described.

Chemistry General Methods

All reactions were performed with commercially available reagents (Aldrich, Thermo-Fisher, Alfa Aesar, Acros, Oakwood, Millipore) without further purification. Anhydrous solvents were acquired through a solvent purification system (Inert PureSolv and JC Meyer systems) or purified according to standard procedures. ¹H NMR spectra were recorded on Bruker DRX 400 MHz, Bruker DRX 500 MHz, or Bruker Avance 600 MHz spectrometer and processed using TopSpin 3.5 software. LRMS and HRMS data were obtained via ESI LC-TOF on a Waters (Micromass) LCT Premier spectrometer (Waters), with PEG as the calibrant for HRMS. Optical rotation was recorded on a Rudolph Autopol III Automatic Polarimeter at room temperature in methanol. TLC was performed using EMD Millipore silica gel 60 F₂₅₄ aluminum plates. Separation by column chromatography was conducted using Fisher silica gel 60 (230-400 mesh). All investigated compounds were >95% pure as determined by NMR. High resolution mass spectrometry data and NMR spectra are included in the Supplementary Material.

### Synthesis of Analogues

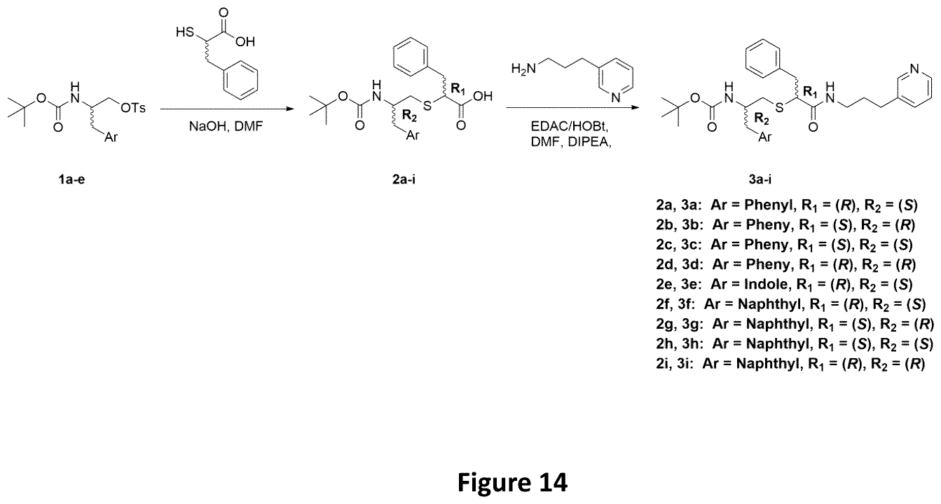

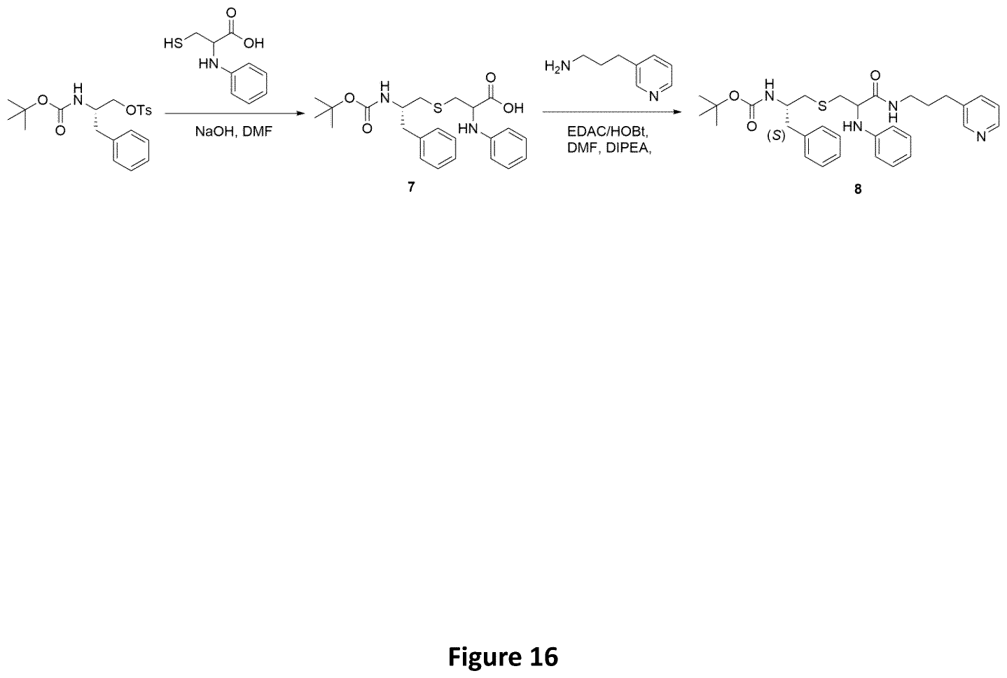

Compounds 1a-e were prepared as described previously (Samuels E R et al., 2019, Biochemistry, 58:2077-2087; Samuels E R et al., 2020, Bioorg. Med. Chem., 28:115349-115360), with either boc-protection and tosylation of commercially available amino alcohol or amino acid reduction and protection. Sequentially, the Ar groups are: compound 1a phenyl (S), compound 1b phenyl (R), compound 1c indole (S), compound 1d naphthyl (S), and compound 1e naphthyl (R). Compounds 2a-i were also prepared as described previously (Samuels E R et al., 2019, Biochemistry, 58:2077-2087; Samuels E R et al., 2020, Bioorg. Med. Chem., 28:115349-115360), with either D or L-α-thio-phenylalanine (Samuels E et al., 2018, Tetrahedron lett., 59:1140-1142). Synthesis of other analogues is outlined in FIG. 14 through FIG. 16.

### General Procedure for Synthesis of Compounds 3a-i

Crude compound 2a (0.34 g, 0.82 mmol) was dissolved in DMF (3 mL). To this solution, 3-(3-pyridyl)propylamine (0.17 g, 1.23 mmol, 1.5 eq) in 3 ml DMF was added, followed by EDAC (0.24 g, 1.23 mmol, 1.5 eq), HOBt (0.19 g, 1.23 mmol, 1.5 eq) and DIPEA (0.32 g, 2.46 mmol, 3 eq). The reaction was stirred at room temperature overnight. Upon completion, the solvent was evaporated and the reaction mixture was diluted with ethyl acetate. The organic layer was then washed with saturated NaHCO₃, water, and brine. The combined organic layers were dried over MgSO₄ and concentrated in vacuo to give the crude product, which was purified via column chromatography (95:5 EtOAc:MeOH).

The pure product 3a was obtained as a clear yellow oil (0.07 g, 16%). TLC: EtOAc/MeOH 90:10 (Rf. 0.57). ¹H NMR (400 MHz, CDCl₃) δ 8.43 (d, J=4.8 Hz, 1H), 8.37 (s, 1H), 7.43 (d, J=7.7 Hz, 1H), 7.36-7.11 (m, 11H), 6.52 (t, J=6.0 Hz, 1H(NH)), 4.63 (d, J=8.3 Hz, 1H), 3.96 (m, 1H), 3.25 (quint, J=6.7 Hz, 2H), 2.90 (m, 2H), 2.79 (d, J=6.9 Hz, 2H), 2.68 (dd, J=5.2, 13.6 Hz, 1H), 2.58 (dd, J=6.1, 13.5 Hz, 1H), 2.48 (t, J=7.9 Hz, 2H), 1.69 (quint, J=7.3 Hz, 2H), 1.40 (s, 9H). HRMS m z calculated for C₃₁H₃₉N₃O₃SNa [M+Na]⁺: 556.2610. Found: 556.2606.

The pure product 3b was obtained as a yellow gum (0.015 g, 15%). TLC: EtOAc/MeOH 90:10 (Rf. 0.57). ¹H NMR (400 MHz, CDCl₃) δ 8.45 (d, J=4.9 Hz, 1H), 8.39 (s, 1H), 7.44 (d, J=8.1 Hz, 1H), 7.31-7.16 (m, 9H), 7.13 (d, J=6.8 Hz, 2H), 6.45 (t, J=7.0 Hz, 1H(NH)), 4.57 (bs, 1H), 3.96 (m, 1H), 3.26 (quint, J=7.0 Hz, 2H), 3.18 (m, 1H), 2.96 (dd, J=7.0, 13.4 Hz, 1H), 2.79 (d, J=7.0 Hz, 2H), 2.67 (dd, J=5.7 13.5 Hz, 1H), 2.58 (dd, J=6.0, 13.4 Hz, 1H), 2.49 (t, J=7.7 Hz, 2H), 1.70 (quint, J=7.4 Hz, 2H), 1.41 (s, 9H). HRMS m z calculated for C₃₁H₃₉N₃O₃SNa [M+Na]⁺: 556.2610. Found: 556.2587.

The pure product 3c was obtained as a yellow gum (0.018 g, 18%). TLC: EtOAc/MeOH 90:10 (Rf. 0.57). ¹H NMR (400 MHz, CDCl₃) δ 8.44 (d, J=4.8 Hz, 1H), 8.39 (s, 1H), 7.45 (d, J=7.6 Hz, 1H), 7.32-7.17 (m, 9H), 7.08 (d, J=6.7 Hz, 2H), 6.90 (bs, 1H (NH)), 4.64 (d, J=6.9 Hz, 1H), 3.85 (q, J=6.7 Hz, 1H), 3.54 (m, 1H), 3.30 (dd, J=7.4, 13.7 Hz, 2H), 3.19 (quint, J=6.7 Hz, 1H), 2.91 (m, 2H), 2.68 (m, 2H), 2.50 (m, 2H), 1.73 (quint, J=7.5 Hz, 2H), 1.37 (s, 9H). HRMS m z calculated for C₃₁H₃₉N₃O₃SNa [M+Na]⁺: 556.2610. Found: 556.2585.

The pure product 3d was obtained as a yellow gum (0.027 g, 21%). TLC: EtOAc/MeOH 90:10 (Rf. 0.57). ¹H NMR (400 MHz, CDCl₃) δ ¹H NMR (400 MHz, CDCl₃) δ 8.44 (d, J=4.8 Hz, 1H), 8.37 (s, 1H), 7.45 (d, J=7.8 Hz, 1H), 7.31-7.17 (m, 9H), 7.08 (d, J=6.8 Hz, 2H), 6.89 (bs, 1H(NH)), 4.64 (d, J=7.8 Hz, 1H), 3.85 (q, J=7.0 Hz, 1H), 3.54 (m, 1H), 3.30 (dd, J=7.3, 13.7 Hz, 2H), 3.19 (quint, J=6.7 Hz, 1H), 2.91 (m, 2H), 2.69 (m, 2H), 2.49 (m, 2H), 1.73 (quint, J=7.4 Hz, 2H), 1.39 (s, 9H). HRMS m z calculated for C₃₁H₃₉N₃O₃SNa [M+Na]⁺: 556.2610. Found: 556.2598.

Compound 3e was obtained as a white fluffy solid (0.022 g, 13%). TLC: EtOAc/MeOH 90:10 (Rf. 0.49). ¹H NMR (400 MHz, CDCl₃) δ 8.44 (d, J=4.8 Hz, 1H), 8.33 (s, 1H). 8.30 (s, 1H), 7.61 (d, J=7.6 Hz, 1H), 7.41 (d, J=7.8 Hz, 1H), 7.38-7.13 (m, 7H), 7.10 (t, J=6.9 Hz, 1H), 6.98 (s, 1H), 6.33 (t, J=5.2 Hz, 1H(NH)), 4.69 (d, J=7.7 Hz, 1H), 4.09 (q, J=6.6 Hz, 1H), 3.25 (dd, J=7.4, 13.7 Hz, 1H), 3.12 (quint, J=6.8 Hz, 1H), 2.97 (m, 4H), 2.71 (dd, J=5.5, 13.9 Hz, 1H), 2.59 (dd, J=6.2, 13.4 Hz, 1H), 2.40 (t, J=7.7 Hz, 2H), 1.56 (quint, J=7.4 Hz, 2H), 1.43 (s, 9H). HRMS m z calculated for C₃₃H₄₁N₄O₃S [M+H]⁺: 573.2899. Found: 573.2900.

The pure product 3f was obtained as an off white fluffy solid (0.031 g, 20%). TLC: EtOAc/MeOH 90:10 (Rf. 0.51). ¹H NMR (400 MHz, CDCl₃) δ 8.43 (d, J=4.7 Hz, 1H), 8.13 (d, J=8.4 Hz, 1H), 7.82 (d, J=8.0 Hz, 1H), 7.72 (d, J=8.1 Hz, 1H), 7.55 (t, J=7.8 Hz, 1H), 7.47 (t, J=8.0 Hz, 1H), 7.36 (m, 3H), 7.31-7.16 (m, 7H), 6.34 (t, J=5.3 Hz, 1H(NH)), 4.73 (d, J=7.9 Hz, 1H), 4.16 (q, J=6.3 Hz, 1H), 3.25 (m, 4H), 2.96 (dd, J=6.8, 13.7 Hz, 2H), 2.68 (t, J=4.6 Hz, 2H), 2.36 (t, J=7.8 Hz, 2H), 1.53 (t, J=7.4 Hz, 2H), 1.39 (s, 9H). HRMS m z calculated for C₃₅H₄₂N₃O₃S [M+H]⁺: 584.2947. Found: 584.2921.

The pure product 3g was obtained as a yellow solid (0.024 g, 16%). TLC: EtOAc/MeOH 90:10 (Rf. 0.5). ¹H NMR (400 MHz, CDCl₃) δ 8.44 (d, J=4.7 Hz, 1H), 8.13 (d, J=8.3 Hz, 1H), 7.82 (d, J=8.2 Hz, 1H), 7.72 (d, J=8.0 Hz, 1H), 7.55 (t, J=7.6 Hz, 1H), 7.47 (t, J=8.0 Hz, 1H), 7.36 (m, 3H), 7.31-7.16 (m, 7H), 6.32 (t, J=6.8 Hz, 1H(NH)), 4.72 (d, J=8.0 Hz, 1H), 4.16 (q, J=6.5 Hz, 1H), 3.25 (m, 4H), 2.96 (dd, J=6.8, 13.7 Hz, 2H), 2.68 (t, J=4.8 Hz, 2H), 2.36 (t, J=6.8 Hz, 2H), 1.54 (t, J=6.7 Hz, 2H), 1.39 (s, 9H). HRMS m z calculated for C₃₅H₄₂N₃O₃S [M+H]⁺: 584.2947. Found: 584.2930.

The pure product 3h was obtained as an off white fluffy solid (0.089 g, 26%). TLC: EtOAc/MeOH 90:10 (Rf. 0.51 (Avg)). ¹H NMR (400 MHz, CDCl₃) δ 8.43 (d, J=4.8 Hz, 1H), 8.35 (s, 1H), 8.16 (d, J=7.8 Hz, 1H), 7.88 (d, J=7.6 Hz, 1H), 7.77 (d, J=8.2 Hz, 1H), 7.53 (quint, J=7.6 Hz, 2H), 7.40 (t, J=7.5 Hz, 2H), 7.29-7.16 (m, 7H), 6.81 (t, J=5.3 Hz, 1H(NH)), 4.77 (d, J=7.1 Hz, 1H), 4.08 (q, J=6.4 Hz, 1H), 3.53 (m, 1H), 3.30 (m, 2H), 3.11 (m, 2H), 2.93 (dd, J=6.0, 13.6 Hz, 1H), 2.79 (m, 1H), 2.54 (dd, J=6.6, 13.9 Hz, 1H), 2.43 (sext, J=6.3 Hz, 2H), 1.67 (oct, J=7.4 Hz, 2H), 1.34 (s, 9H). HRMS m z calculated for C₃₅H₄₂N₃O₃S [M+H]⁺: 584.2947. Found: 584.2928.

The pure product 3i was obtained as a white fluffy solid (0.07 g, 26%). TLC: EtOAc/MeOH 90:10 (Rf. 0.49). ¹H NMR (400 MHz, CDCl₃) δ 8.43 (d, J=4.7 Hz, 1H), 8.35 (s, 1H), 8.16 (d, J=7.8 Hz, 1H), 7.86 (dd, J=8.2, 16.5 Hz, 1H), 7.75 (dd, J=8.0, 19.1 Hz, 1H), 7.53 (m, J=7.7 Hz, 2H), 7.40 (t, J=7.7 Hz, 2H), 7.30-7.15 (m, 7H), 6.82 (bs, 1H(NH)), 4.77 (d, J=8.0 Hz, 1H), 4.08 (sext, J=6.7 Hz, 1H), 3.53 (m, 1H), 3.31 (dd, J=8.5, 13.5 Hz, 2H), 3.13 (dd, J=8.0, 14.8 Hz, 2H), 2.93 (dd, J=6.2, 13.5 Hz, 1H), 2.79 (m, 1H), 2.54 (dd, J=6.9, 14.3 Hz, 1H), 2.43 (sext, J=5.9 Hz, 2H), 1.67 (sext, J=7.6 Hz, 2H), 1.34 (s, 9H). HRMS m z calculated for C₃₅H₄₁N₃O₃SNa [M+Na]⁺: 606.2766. Found: 606.2797.

### Synthesis of Compound 4

Pyridine-3-methanol (2.0 g, 18.3 mmol) was dissolved in anhydrous DMF (30 mL) and the solution was cooled to 0° C. on an ice bath. Sodium hydride (0.88 g, 36.6 mmol, 2 eq) was added slowly over 10 minutes. Once H₂ formation subsided, the solution was warmed to 60° C. and stirred for 45 minutes. The solution was then cooled to 0° C. on an ice bath and bromoacetonitrile (2.74 g, 22.8 mmol, 1.25 eq) was added dropwise. The reaction was allowed to slowly come to room temperature overnight. Upon completion, the solvent was evaporated, and the reaction mixture was diluted with ethyl acetate. The organic layer was then washed with saturated NaHCO₃, water, and brine. The combined organic layers were dried over MgSO₄, filtered through a silica plug, and concentrated in vacuo to give the crude product, which was purified via column chromatography (100% EtOAc). The pure product 4 was obtained as a brown oil (0.65 g, 24%). LRMS m/z calculated for C₈H₈N₂ONa [M+Na]⁺: 171.0. Found: 171.0.

### Synthesis of Compound 5

Compound 4 (0.5 g, 3.4 mmol) was dissolved in methanol (20 mL) and the solution was cooled to 0° C. on an ice bath. Nickel chloride hexahydrate (0.89 g, 3.7 mmol, 1.1 eq,) and Di-tert-butyl dicarbonate (1.5 g, 6.8 mmol, 2 eq) were then added to the solution, followed by slow addition of sodium borohydride (1.3 g, 34 mmol, 10 eq) over 10 min. Once H₂ formation subsided, the solution was removed from the ice bath and allowed to stir at room temperature overnight. Upon completion, the solvent was evaporated, and the reaction mixture was diluted with ethyl acetate. The organic layer was then washed with saturated NaHCO₃ and filtered through celite. The organic layers were dried over MgSO₄ and concentrated in vacuo to give the crude product, which was purified via column chromatography (100% EtOAc). The pure product 5 was obtained as a brown oil (0.1 g, 12%). LRMS m/z calculated for C₁₃H₂₀N₂O₃Na [M+Na]⁺: 275.1. Found: 275.1.

### Synthesis of Compound 6

First, compound 5 (0.1 g, 0.40 mmol) was added to DCM (3 mL). Trifluoroacetic acid (1.5 mL) was added dropwise and the reaction was allowed to stir at room temperature for 2 hours. Upon completion, the solvent was evaporated, and the reaction mixture was neutralized with NaHCO₃. The pH was then adjusted to 14 with NaOH, and the solution was extracted with DCM. The combined organic layers were concentrated in vacuo to give the free amine product (2-(pyridine-3-ylmethoxy)ethan-1-amine) as a light-yellow oil (0.06 g, 98%). HRMS m/z calculated for C₈H₁₃N₂O [M+H]⁺: 153.1028. Found: 153.1043. Next, crude 2a (0.15 g, 0.36 mmol) was dissolved in DMF (3 ml). To this solution, EDAC (0.1 g, 0.54 mmol, 1.5 eq) and HOBt (0.083 g, 0.54 mmol, 1.5 eq) were added, followed by the addition of 2-(pyridine-3-ylmethoxy)ethan-1-amine (0.06 g, 0.39 mmol, 1.1 eq) in DMF (2 ml) and DIPEA (0.14 g, 1.1 mmol, 3 eq). The reaction was stirred at room temperature overnight. Upon completion, the solvent was evaporated, and the reaction mixture was diluted with ethyl acetate. The organic layer was then washed with saturated NaHCO₃, water, and brine. The combined organic layers were dried over MgSO₄ and concentrated in vacuo to give the crude product, which was purified via column chromatography (100% EtOAc). The pure product 6 was obtained as a light orange oil (0.013 g, 6.5%). TLC: EtOAc/MeOH 90:10 (Rf. 0.54). ¹H NMR (400 MHz, CDCl₃) δ 8.54 (m, 2H), 7.64 (d, J=6.2 Hz, 1H), 7.34-7.07 (m, 11H), 6.75 (m, 1H(NH)), 4.43 (s, 1H), 3.64 (m, 2H), 3.47 (m, 2H), 3.36 (s, 1H), 3.25 (dd, J=6.9, 13.4 Hz, 1H), 2.95 (m, 2H), 2.73 (m, 2H), 2.65 (d, J=6.8, 1H), 2.54 (dd, J=6.5, 13.9 Hz, 1H), 2.35 (dd, J=4.5, 19.4 Hz, 1H), 1.41 (s, 9H). HRMS m z calculated for C₃₁H₄₀N₃O₄S [M+H]⁺: 550.2739. Found: 550.2738.

### Synthesis of Compound 7

Compound 7 was prepared as described previously (Samuels E R et al., 2018, Mol. Pharm., 15:279-288), with n-phenylcysteine and Boc-protected, tosylated L-phenylalanine (Boc-L-Phe-OTs).

### Synthesis of Compound 8

Crude 7 (0.34 g, 0.8 mmol) was dissolved in DMF (9 mL). To this solution, EDAC (0.23 g, 1.2 mmol, 1.5 eq) and HOBt (0.18 g, 1.2 mmol, 1.5 eq) were added, followed by the addition of 3-(3-pyridyl)propylamine (0.16 g, 1.2 mmol, 1.5 eq) and DIPEA (0.31 g, 2.4 mmol, 3 eq). The reaction was stirred at room temperature overnight. Upon completion, the solvent was evaporated, and the reaction mixture was diluted with ethyl acetate. The organic layer was then washed with saturated NaHCO₃, water, and brine. The combined organic layers were dried over MgSO₄ and concentrated in vacuo to give the crude product, which was purified via column chromatography (95:5 EtOAc:MeOH). The pure product 8 was acquired as a light-yellow gum (0.032 g, 23%). TLC: EtOAc/MeOH 90:10 (Rf. 0.55). ¹H NMR (400 MHz, CDCl₃) δ 8.43 (d, J=4.7 Hz, 1H), 8.36 (s, 1H), 7.41 (m, 2H), 7.30-7.12 (m, 6H), 7.00 (t, J=6.1 Hz, 1H), 6.85 (dt, J=3.4, 11.0 Hz, 1H), 6.67 (t, J=8.5 Hz, 2H), 4.70 (t, J=10.6 Hz, 1H), 4.61 (bs, 1H(NH)), 4.00 (bs, 1H), 3.86 (bd, J=28.8 Hz, 1H(NH)), 3.30 (m, 2H), 3.15 (m, 1H), 3.00 (m, 1H), 2.85 (m, 2H), 2.64 (m, 2H), 2.56 (t, J=7.7 Hz, 2H), 1.87 (bs, 1H(NH)), 1.78 (m, 2H), 1.42 (d, J=3.0 Hz, 9H). HRMS m/z calculated for C₃₁H₄₀N₄O₃SNa [M+Na]⁺: 571.2719. Found: 571.2698.

Protein Expression and Purification

Codon-optimized full-length and Δ3-22 human CYP3A4 were produced as reported previously (Sevrioukova I F, 2017, Biochemistry, 56:3058-3067) and used for assays and crystallization, respectively. The K421A and K424A mutations were introduced to the Δ3-22 CYP3A4 expression plasmid using a QuikChange mutagenesis kit (Stratagene) and confirmed by sequencing. The mutant was expressed and purified similar to the wild type (WT) protein.

Spectral Binding Titrations

Equilibrium ligand binding to CYP3A4 was monitored in a Cary 300 spectrophotometer at ambient temperature in 0.1 M phosphate buffer, pH 7.4, supplemented with 20% glycerol and 1 mM dithiothreitol. Inhibitors were dissolved in dimethyl sulfoxide (DMSO) and added to a 2 μM protein solution in small aliquots, with the final solvent concentration<2%. Spectral dissociation constants (Kₛ) were determined from quadratic fits to titration plots.

Thermal Denaturation

Thermal denaturation curves were recorded in 0.1 M phosphate buffer, pH 7.4, in a Cary 300 spectrophotometer. Protein (1 μM) was mixed with a ligand (20 μM) and incubated for 15 min at 23° C. Melting curves were recorded at 260 nm using a 0.2° C. measurement step, 0.9° C./min ramp rate, and 50-75° C. temperature range. A denaturation midpoint (melting temperature; Tₘ) was determined from non-linear fittings to the melting curve as described earlier (Samuels E R et al., 2019, Biochemistry, 58:2077-2087).

Inhibitory Potency Assays

Inhibitory potency for the 7-benzyloxy-4-(trifluoromethyl)coumarin (BFC)O-debenzylation activity of CYP3A4 was evaluated fluorometrically in a soluble reconstituted system. The full-length CYP3A4 and rat cytochrome P450 reductase (40 μM and 60 μM, respectively) were preincubated at room temperature for 1 hour before 20-fold dilution with the reaction buffer consisting of 0.1 M potassium phosphate, pH 7.4, catalase and superoxide dismutase (2 Units/ml each), and 0.0025% CHAPS (3-[(3-cholamidopropyl)dimethyl-ammonio]-1-propanesulfonate). Prior to measurements, 85 μL of the reaction buffer was mixed with 10 μL of the NADPH-regenerating system (10 mM glucose, 0.2 mM NADP+, and 2 Units/ml glucose-6-phosphate dehydrogenase), 5 μL of the protein mixture (0.1 μM final CYP3A4 concentration), and 2 μL of the inhibitor solution or DMSO. The mixture was incubated for 2 min, after which 20 μL BFC and 70 μL NADPH (final concentration) were added to initiate the reaction. Accumulation of the fluorescent product, 7-hydroxy-4-(trifluoromethyl)coumarin, was monitored for 2 min at room temperature in a Hitachi F400 fluorimeter (λₑₓ=404 nm; λₑₘ=500 nm). Within this time interval, fluorescence changes were linear. The average of three measurements was used to calculate the remaining activity, with the DMSO-containing sample used as a control (100% activity). The IC₅₀ values were derived from the [% activity] vs. [inhibitor] plots as described previously (Samuels E R et al., 2019, Biochemistry, 58:2077-2087). Prior work showed that preincubation of ritonavir or the analogues with NADPH in a lipid-free reconstituted system leads to a small increase rather than decrease in IC₅₀, likely to due to partial metabolism (Samuels E R et al., 2019, Biochemistry, 58:2077-2087). Therefore, in this study, inhibitory assays with NADPH preincubation were not conducted.

Kinetics of Ligand Binding and Heme Reduction

Kinetics of ligand binding to CYP3A4 and its reduction with sodium dithionite were measured at 426 nm and 443 nm, respectively, in a SX.18MV stopped flow apparatus (Applied Photophysics, UK), as described earlier (Samuels E R et al., 2019, Biochemistry, 58:2077-2087).

H₂O₂-Dependent Heme Depletion Assay

Heme bleaching in ligand-free and inhibitor-bound CYP3A4 (1.6 μM) was monitored at ambient temperature in 0.1 M phosphate buffer, pH 7.4. After 10 min preincubation of CYP3A4 with 16 μM inhibitors, 10 mM H₂O₂ (final concentration) was added, and heme decay was monitored at 420 nm for 120 min. The percentage of heme destroyed at the end of the reaction was calculated relative to that in the ligand-free CYP3A4 (100% decay).

Crystallization of the Inhibitory Complexes

Compounds 3a-d, 8, and 3e-h were co-crystallized with WTΔ3-22 CYP3A4, and compounds 3d and 3i with the K421A/K424A mutant. Crystals of 3b-, 3c-, 3f-, 3g-, and 8-bound complexes were grown using a microbatch method under paraffin oil. Other compounds were co-crystallized with CYP3A4 using a sitting drop vapor diffusion method. Prior to crystallization setup, CYP3A4 (60-70 mg/ml in 20-100 mM phosphate, pH 7.4) was incubated with a 2-fold ligand excess and centrifuged to remove the precipitate. The supernatant (0.4-0.6 μL) was mixed with 0.4-0.6 μL of crystallization solution containing: 8-10% polyethylene glycol (PEG) 3350 and 70-80 mM sodium malonate, pH 7.0, for compounds 3a, 3g, and 8; 10-12% PEG 3350 and 60-100 mM succinate, pH 7.0, for compounds 3b and 3f; 10% PEG 3350 and 80 mM taximate, pH 6.0 (Hampton Research) for 3c; 6% PEG 3350 and 50 mM sodium malonate, pH 7.0, for compound 3e; or 6-8% PEG 4000, 50 mM HEPES, pH 8.0, and 0.1-0.2 M lithium sulfate for compounds 3d, 3h, and 3i. Crystals were grown at room temperature for 2-3 days and cryoprotected with Paratone-N before freezing in liquid nitrogen.

Determination of the X-Ray Structures

X-ray diffraction data were collected at the Stanford Synchrotron Radiation Lightsource beamlines 9-2 and 12-2, and the Advanced Light Source beamlines 5.0.2 and 8.2.2. Crystal structures were solved by molecular replacement with PHASER (McCoy A J et al., 2007, J. Appl. Crystallogr., 40:658-674). 5VCC was used as a search model for the 3b-, 3c-, 3f-, 3g- and 8-bound complexes, crystallized in the I222 space group. For the 3a-, 3d-, 3h- and 3i-bound CYP3A4, crystallized in the C2 and I2 space groups, the search model was the CYP3A4-3e dimer, whose structure was determined first using 6UNJ for molecular replacement. Ligands were built with eLBOW (Adams P D et al., 2010, Acta Crystallogr. Section D, 66:213-321) and manually fit into the density with COOT (Emsley P et al., 2010, Acta Crystallogr. Section D, 66:486-501). The initial models were rebuilt and refined with COOT and PHENIX (Emsley P et al., 2010, Acta Crystallogr. Section D, 66:486-501). For racemic compound 8, the R₁ side-group configuration was automatically assigned to R during the first refinement cycle. The following refinement was conducted with all stereoisomer combinations to confirm that the assigned chirality was most optimal. Polder omit electron density maps were calculated with PHENIX. Data collection and refinement statistics are summarized in FIG. 20 and FIG. 21. The atomic coordinates and structure factors for the 3a-, 3b-, 3c-, 3d-, 8-, 3e-, 3f-, 3g-, 3h-, and 3i-bound CYP3A4 were deposited in the Protein Data Bank with the ID codes 7KVH, 7KVI, 7KVJ, 7KVK, 7KVM, 7KVN, 7KVO, 7KVP, 7KVQ and 7KVS, respectively.

Additional data demonstrated that reorganization of the F-F′ fragment in C2 crystals induced by different packing with a view at the distal face of WT and K421A/K424A CYP3A4, comparison of the compounds 3a and 3h binding modes in I222 and C2 crystals, absolute spectra of ferric and ferrous ligand-free and 3d-, 3i- and ritonavir-bound CYP3A4 K421A/K2424A with the respective titrations plots, X-ray data collection and refinement statistics, ¹H NMR spectra, and high resolution mass spectrometry data.

The disclosures of each and every patent, patent application, and publication cited herein are hereby incorporated herein by reference in their entirety. While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound having the structure of Formula (I): 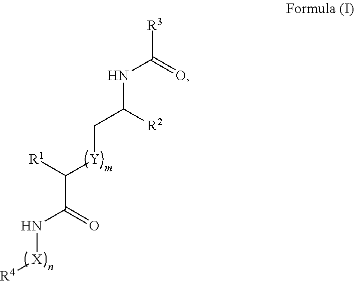
a salt or solvate thereof, and any combinations thereof;
wherein R₁ and R₂ are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, —(C₁-C₆)alkyl-aryl, substituted —(C₁-C₆)alkyl-aryl, —(C₁-C₆)alkyl-phenyl, substituted —(C₁-C₆)alkyl-phenyl, —(C₁-C₆)alkyl-carbocyclic, —(C₁-C₆)alkyl-heteroaryl, substituted —(C₁-C₆)alkyl-heteroaryl, N(R₅)(R₆), and any combination thereof;
R₃ is selected from the group consisting of heteroaryl, substituted heteroaryl, heterocyclyl, substituted heterocyclyl, —(C₁-C₆)alkyl-NHC(═O)R₇, and any combination thereof;
R₄ is selected from the group consisting of heteroaryl, substituted heteroaryl, heterocyclyl, substituted heterocyclyl, and any combination thereof;
Wherein R₅ and R₆ are each independently selected from the group consisting of H, C₁-C₆ alkyl, substituted C₁-C₆ alkyl, aryl, phenyl, substituted phenyl, heteroaryl, substituted heteroaryl, heterocyclyl, substituted heterocyclyl, —(C₁-C₆)alkyl-aryl, substituted —(C₁-C₆)alkyl-aryl, —(C₁-C₆)alkyl-phenyl, substituted —(C₁-C₆)alkyl-phenyl, —(C₁-C₆)alkyl-carbocyclic, —(C₁-C₆)alkyl-heteroaryl, substituted-(C₁-C₆)alkyl-heteroaryl, and any combination thereof; and
R₇ is selected from the group consisting of phenyl, substituted phenyl, heteroaryl, substituted heteroaryl, heterocyclyl, substituted heterocyclyl, and any combination thereof,
each occurrence of Y is independently selected from the group consisting of CH₂, NH, S, O, and any combination thereof,
each occurrence of X is independently selected from the group consisting of —(C₁-C₆) alkyl, —(C₁-C₆) alkyl-O—(C₁-C₆) alkyl-, and any combination thereof;
m is an integer from 0 to 1; and
n is an integer from 0 to 3.

2. The compound of claim 1, wherein R₁ is benzyl.

3. The compound of claim 1, wherein R₂ is selected from the group consisting of —(C₁-C₆)alkyl-aryl and —(C₁-C₆)alkyl-heteroaryl.

4. The compound of claim 1, wherein R₃ is selected from the group consisting of furan, thiophene, benzofuran, benzothiophene, and pyridine.

5. The compound of claim 1, wherein R₄ is pyridine.

6. The compound of claim 1, wherein R₇ is pyridine.

7. The compound of claim 1, wherein m is 1.

8. The compound of claim 1, wherein n is 1.

9. The compound of claim 1, wherein Y is S.

10. The compound of claim 1, wherein the compound is selected from the group consisting of: 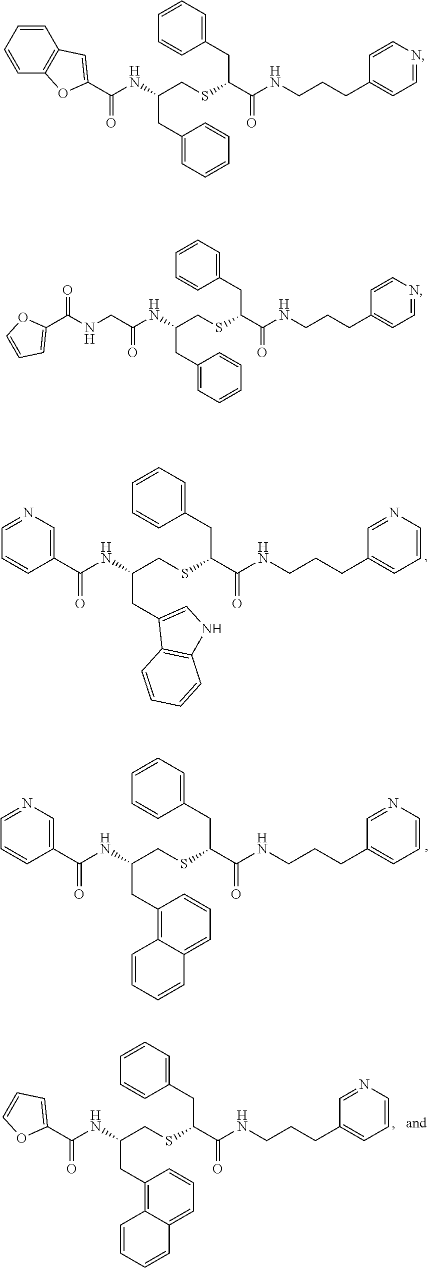 [SMILES: O=C(N[C@H](CS[C@H](Cc1ccccc1)C(=O)NCCCc1ccncc1)Cc1ccccc1)c1cc2ccccc2o1 ; O=C(CNC(=O)c1ccco1)N[C@H](CS[C@H](Cc1ccccc1)C(=O)NCCCc1ccncc1)Cc1ccccc1 ; O=C(N[C@H](CS[C@H](Cc1ccccc1)C(=O)NCCCc1cccnc1)Cc1c[nH]c2ccccc12)c1cccnc1 ; O=C(N[C@H](CS[C@H](Cc1ccccc1)C(=O)NCCCc1cccnc1)Cc1cccc2ccccc12)c1cccnc1 ; O=C(N[C@H](CS[C@H](Cc1ccccc1)C(=O)NCCCc1cccnc1)Cc1cccc2ccccc12)c1ccco1] 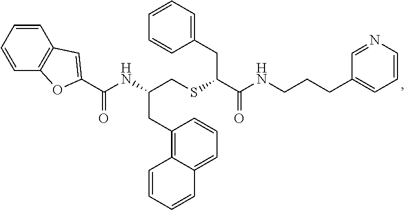 [SMILES: O=C(N[C@H](CS[C@H](Cc1ccccc1)C(=O)NCCCc1cccnc1)Cc1cccc2ccccc12)c1cc2ccccc2o1]
a salt or solvate thereof, and any combinations thereof.

11. A composition comprising a compound of claim 1.

12. The composition of claim 11, wherein the composition further comprises a pharmaceutically acceptable carrier.

13. A method of inhibiting at least one cytochrome P450 3A4 (CYP3A4) in a subject in need thereof, the method comprising administering to the subject a therapeutically effective amount of at least one compound of claim 1 or a composition thereof.

14. The method of claim 13, wherein the compound is selected from the group consisting of: 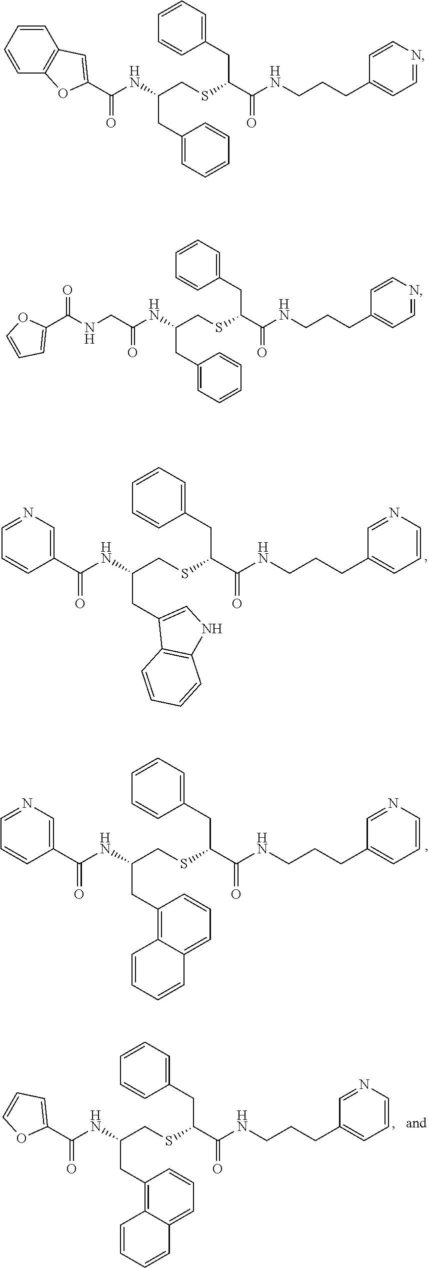 [SMILES: O=C(N[C@H](CS[C@H](Cc1ccccc1)C(=O)NCCCc1ccncc1)Cc1ccccc1)c1cc2ccccc2o1 ; O=C(CNC(=O)c1ccco1)N[C@H](CS[C@H](Cc1ccccc1)C(=O)NCCCc1ccncc1)Cc1ccccc1 ; O=C(N[C@H](CS[C@H](Cc1ccccc1)C(=O)NCCCc1cccnc1)Cc1c[nH]c2ccccc12)c1cccnc1 ; O=C(N[C@H](CS[C@H](Cc1ccccc1)C(=O)NCCCc1cccnc1)Cc1cccc2ccccc12)c1cccnc1 ; O=C(N[C@H](CS[C@H](Cc1ccccc1)C(=O)NCCCc1cccnc1)Cc1cccc2ccccc12)c1ccco1] 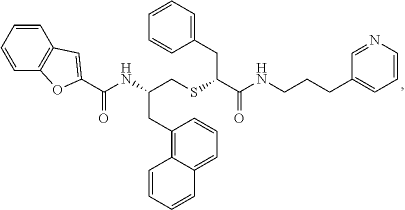 [SMILES: O=C(N[C@H](CS[C@H](Cc1ccccc1)C(=O)NCCCc1cccnc1)Cc1cccc2ccccc12)c1cc2ccccc2o1]
a salt or solvate thereof, and any combinations thereof.

15. The method of claim 13, wherein the method further comprises administering to the subject at least one additional therapeutic agent.

16. The method of claim 15, wherein the therapeutic agent is selected from the group consisting of an antiviral agent, anti-cancer agent, immunosuppressant agent, and any combination thereof.

17. The method of claim 15, wherein the composition and the additional therapeutic agent are co-administered.

18. The method of claim 15, wherein the composition and the additional therapeutic agent are co-formulated.

19. The method of claim 15, wherein the therapeutic agent is a protease inhibitor.

20. A method of treating or preventing at least one disease or disorder associated with cytochrome P450 3A4 (CYP3A4) in a subject in need thereof, the method comprising administering to the subject a therapeutically effective amount of at least one compound of claim 1 or a composition thereof.
